SEQUENCE LISTING (282 sequences)

SEQ ID NO: 1 - DNA, 32 bp, Artificial Sequence
  Description of Artificial Sequence Synthetic oligonucleotide
tggtttttaacgtcaaagggcgaagaaccatc

SEQ ID NO: 2 - DNA, 32 bp, Artificial Sequence
  Description of Artificial Sequence Synthetic oligonucleotide
cttgcatgcattaatgaatcggcccgccaggg

SEQ ID NO: 3 - DNA, 32 bp, Artificial Sequence
  Description of Artificial Sequence Synthetic oligonucleotide
tagatggggggtaacgccagggttgtgccaag

SEQ ID NO: 4 - DNA, 32 bp, Artificial Sequence
  Description of Artificial Sequence Synthetic oligonucleotide
catgtcaagattctccgtgggaaccgttggtg

SEQ ID NO: 5 - DNA, 32 bp, Artificial Sequence
  Description of Artificial Sequence Synthetic oligonucleotide
ctgtaatattgcctgagagtctggaaaactag

SEQ ID NO: 6 - DNA, 32 bp, Artificial Sequence
  Description of Artificial Sequence Synthetic oligonucleotide
tgcaactaagcaataaagcctcagttatgacc

SEQ ID NO: 7 - DNA, 32 bp, Artificial Sequence
  Description of Artificial Sequence Synthetic oligonucleotide
aaacagttgatggcttagagcttatttaaata

SEQ ID NO: 8 - DNA, 32 bp, Artificial Sequence
  Description of Artificial Sequence Synthetic oligonucleotide
acgaactagcgtccaatactgcggaatgcttt

SEQ ID NO: 9 - DNA, 32 bp, Artificial Sequence
  Description of Artificial Sequence Synthetic oligonucleotide
ctttgaaaagaactggctcattatttaataaa

SEQ ID NO: 10 - DNA, 32 bp, Artificial Sequence
  Description of Artificial Sequence Synthetic oligonucleotide
acggctacttacttagccggaacgctgaccaa

SEQ ID NO: 11 - DNA, 32 bp, Artificial Sequence
  Description of Artificial Sequence Synthetic oligonucleotide
gagaatagcttttgcgggatcgtcgggtagca

SEQ ID NO: 12 - DNA, 32 bp, Artificial Sequence
  Description of Artificial Sequence Synthetic oligonucleotide
acgttagtaaatgaattttctgtaagcggagt

SEQ ID NO: 13 - DNA, 32 bp, Artificial Sequence
  Description of Artificial Sequence Synthetic oligonucleotide
acccaaatcaagttttttggggtcaaagaacg

SEQ ID NO: 14 - DNA, 32 bp, Artificial Sequence
  Description of Artificial Sequence Synthetic oligonucleotide
tggactcccttttcaccagtgagacctgtcgt

SEQ ID NO: 15 - DNA, 32 bp, Artificial Sequence
  Description of Artificial Sequence Synthetic oligonucleotide
gccagctgcctgcaggtcgactctgcaaggcg

SEQ ID NO: 16 - DNA, 32 bp, Artificial Sequence
  Description of Artificial Sequence Synthetic oligonucleotide
attaagttcgcatcgtaaccgtgcgagtaaca

SEQ ID NO: 17 - DNA, 32 bp, Artificial Sequence
  Description of Artificial Sequence Synthetic oligonucleotide
acccgtcgtcatatgtaccccggtaaaggcta

SEQ ID NO: 18 - DNA, 32 bp, Artificial Sequence
  Description of Artificial Sequence Synthetic oligonucleotide
tcaggtcacttttgcgggagaagcagaattag

SEQ ID NO: 19 - DNA, 32 bp, Artificial Sequence
  Description of Artificial Sequence Synthetic oligonucleotide
caaaattaaagtacggtgtctggaagaggtca

SEQ ID NO: 20 - DNA, 32 bp, Artificial Sequence
  Description of Artificial Sequence Synthetic oligonucleotide
tttttgcgcagaaaacgagaatgaatgtttag

SEQ ID NO: 21 - DNA, 32 bp, Artificial Sequence
  Description of Artificial Sequence Synthetic oligonucleotide
actggataacggaacaacattattaccttatg

SEQ ID NO: 22 - DNA, 32 bp, Artificial Sequence
  Description of Artificial Sequence Synthetic oligonucleotide
cgattttagaggacagatgaacggcgcgacct

SEQ ID NO: 23 - DNA, 32 bp, Artificial Sequence
  Description of Artificial Sequence Synthetic oligonucleotide
gctccatgagaggctttgaggactagggagtt

SEQ ID NO: 24 - DNA, 32 bp, Artificial Sequence
  Description of Artificial Sequence Synthetic oligonucleotide
aaaggccgaaaggaacaactaaagctttccag

SEQ ID NO: 25 - DNA, 32 bp, Artificial Sequence
  Description of Artificial Sequence Synthetic oligonucleotide
agctgattacaagagtccactattgaggtgcc

SEQ ID NO: 26 - DNA, 32 bp, Artificial Sequence
  Description of Artificial Sequence Synthetic oligonucleotide
cccgggtactttccagtcgggaaacgggcaac

SEQ ID NO: 27 - DNA, 32 bp, Artificial Sequence
  Description of Artificial Sequence Synthetic oligonucleotide
gtttgagggaaagggggatgtgctagaggatc

SEQ ID NO: 28 - DNA, 32 bp, Artificial Sequence
  Description of Artificial Sequence Synthetic oligonucleotide
agaaaagcaacattaaatgtgagcatctgcca

SEQ ID NO: 29 - DNA, 32 bp, Artificial Sequence
  Description of Artificial Sequence Synthetic oligonucleotide
caacgcaatttttgagagatctactgataatc

SEQ ID NO: 30 - DNA, 32 bp, Artificial Sequence
  Description of Artificial Sequence Synthetic oligonucleotide
tccatatacatacaggcaaggcaactttattt

SEQ ID NO: 31 - DNA, 32 bp, Artificial Sequence
  Description of Artificial Sequence Synthetic oligonucleotide
caaaaatcattgctccttttgataagtttcat

SEQ ID NO: 32 - DNA, 32 bp, Artificial Sequence
  Description of Artificial Sequence Synthetic oligonucleotide
aaagattcagggggtaatagtaaaccataaat

SEQ ID NO: 33 - DNA, 32 bp, Artificial Sequence
  Description of Artificial Sequence Synthetic oligonucleotide
ccaggcgcttaatcattgtgaattacaggtag

SEQ ID NO: 34 - DNA, 32 bp, Artificial Sequence
  Description of Artificial Sequence Synthetic oligonucleotide
tttcatgaaaattgtgtcgaaatctgtacaga

SEQ ID NO: 35 - DNA, 32 bp, Artificial Sequence
  Description of Artificial Sequence Synthetic oligonucleotide
aataataaggtcgctgaggcttgcaaagactt

SEQ ID NO: 36 - DNA, 32 bp, Artificial Sequence
  Description of Artificial Sequence Synthetic oligonucleotide
cgtaacgatctaaagttttgtcgtgaattgcg

SEQ ID NO: 37 - DNA, 32 bp, Artificial Sequence
  Description of Artificial Sequence Synthetic oligonucleotide
gtaaagcactaaatcggaaccctagttgttcc

SEQ ID NO: 38 - DNA, 32 bp, Artificial Sequence
  Description of Artificial Sequence Synthetic oligonucleotide
agtttggagcccttcaccgcctggttgcgctc

SEQ ID NO: 39 - DNA, 32 bp, Artificial Sequence
  Description of Artificial Sequence Synthetic oligonucleotide
actgcccgccgagctcgaattcgttattacgc

SEQ ID NO: 40 - DNA, 32 bp, Artificial Sequence
  Description of Artificial Sequence Synthetic oligonucleotide
cagctggcggacgacgacagtatcgtagccag

SEQ ID NO: 41 - DNA, 32 bp, Artificial Sequence
  Description of Artificial Sequence Synthetic oligonucleotide
ctttcatccccaaaaacaggaagaccggagag

SEQ ID NO: 42 - DNA, 32 bp, Artificial Sequence
  Description of Artificial Sequence Synthetic oligonucleotide
ggtagctaggataaaaatttttagttaacatc

SEQ ID NO: 43 - DNA, 32 bp, Artificial Sequence
  Description of Artificial Sequence Synthetic oligonucleotide
caataaatacagttgattcccaatttagagag

SEQ ID NO: 44 - DNA, 32 bp, Artificial Sequence
  Description of Artificial Sequence Synthetic oligonucleotide
tacctttaaggtctttaccctgacaaagaagt

SEQ ID NO: 45 - DNA, 32 bp, Artificial Sequence
  Description of Artificial Sequence Synthetic oligonucleotide
tttgccagatcagttgagatttagtggtttaa

SEQ ID NO: 46 - DNA, 32 bp, Artificial Sequence
  Description of Artificial Sequence Synthetic oligonucleotide
tttcaactataggctggctgaccttgtatcat

SEQ ID NO: 47 - DNA, 32 bp, Artificial Sequence
  Description of Artificial Sequence Synthetic oligonucleotide
cgcctgatggaagtttccattaaacataaccg

SEQ ID NO: 48 - DNA, 32 bp, Artificial Sequence
  Description of Artificial Sequence Synthetic oligonucleotide
atatattcttttttcacgttgaaaatagttag

SEQ ID NO: 49 - DNA, 32 bp, Artificial Sequence
  Description of Artificial Sequence Synthetic oligonucleotide
gagttgcacgagatagggttgagtaagggagc

SEQ ID NO: 50 - DNA, 32 bp, Artificial Sequence
  Description of Artificial Sequence Synthetic oligonucleotide
tcatagctactcacattaattgcgccctgaga

SEQ ID NO: 51 - DNA, 32 bp, Artificial Sequence
  Description of Artificial Sequence Synthetic oligonucleotide
gaagatcggtgcgggcctcttcgcaatcatgg

SEQ ID NO: 52 - DNA, 32 bp, Artificial Sequence
  Description of Artificial Sequence Synthetic oligonucleotide
gcaaatatcgcgtctggccttcctggcctcag

SEQ ID NO: 53 - DNA, 32 bp, Artificial Sequence
  Description of Artificial Sequence Synthetic oligonucleotide
tatattttagctgataaattaatgttgtataa

SEQ ID NO: 54 - DNA, 32 bp, Artificial Sequence
  Description of Artificial Sequence Synthetic oligonucleotide
cgagtagaactaatagtagtagcaaaccctca

SEQ ID NO: 55 - DNA, 32 bp, Artificial Sequence
  Description of Artificial Sequence Synthetic oligonucleotide
tcagaagcctccaacaggtcaggatctgcgaa

SEQ ID NO: 56 - DNA, 32 bp, Artificial Sequence
  Description of Artificial Sequence Synthetic oligonucleotide
cattcaacgcgagaggcttttgcatattatag

SEQ ID NO: 57 - DNA, 32 bp, Artificial Sequence
  Description of Artificial Sequence Synthetic oligonucleotide
agtaatcttaaattgggcttgagagaatacca

SEQ ID NO: 58 - DNA, 32 bp, Artificial Sequence
  Description of Artificial Sequence Synthetic oligonucleotide
atacgtaaaagtacaacggagatttcatcaag

SEQ ID NO: 59 - DNA, 32 bp, Artificial Sequence
  Description of Artificial Sequence Synthetic oligonucleotide
aaaaaaggacaaccatcgcccacgcgggtaaa

SEQ ID NO: 60 - DNA, 32 bp, Artificial Sequence
  Description of Artificial Sequence Synthetic oligonucleotide
tgtagcattccacagacagccctcatctccaa

SEQ ID NO: 61 - DNA, 32 bp, Artificial Sequence
  Description of Artificial Sequence Synthetic oligonucleotide
ccccgatttagagcttgacggggaaatcaaaa

SEQ ID NO: 62 - DNA, 32 bp, Artificial Sequence
  Description of Artificial Sequence Synthetic oligonucleotide
gaatagccgcaagcggtccacgctcctaatga

SEQ ID NO: 63 - DNA, 32 bp, Artificial Sequence
  Description of Artificial Sequence Synthetic oligonucleotide
gtgagctagtttcctgtgtgaaatttgggaag

SEQ ID NO: 64 - DNA, 32 bp, Artificial Sequence
  Description of Artificial Sequence Synthetic oligonucleotide
ggcgatcgcactccagccagctttgccatcaa

SEQ ID NO: 65 - DNA, 32 bp, Artificial Sequence
  Description of Artificial Sequence Synthetic oligonucleotide
aaataattttaaattgtaaacgttgatattca

SEQ ID NO: 66 - DNA, 32 bp, Artificial Sequence
  Description of Artificial Sequence Synthetic oligonucleotide
accgttctaaatgcaatgcctgagaggtggca

SEQ ID NO: 67 - DNA, 32 bp, Artificial Sequence
  Description of Artificial Sequence Synthetic oligonucleotide
tcaattcttttagtttgaccattaccagaccg

SEQ ID NO: 68 - DNA, 32 bp, Artificial Sequence
  Description of Artificial Sequence Synthetic oligonucleotide
gaagcaaaaaagcggattgcatcagataaaaa

SEQ ID NO: 69 - DNA, 32 bp, Artificial Sequence
  Description of Artificial Sequence Synthetic oligonucleotide
ccaaaatataatgcagatacataaacaccaga

SEQ ID NO: 70 - DNA, 32 bp, Artificial Sequence
  Description of Artificial Sequence Synthetic oligonucleotide
acgagtagtgacaagaaccggatataccaagc

SEQ ID NO: 71 - DNA, 32 bp, Artificial Sequence
  Description of Artificial Sequence Synthetic oligonucleotide
gcgaaacatgccactacgaaggcatgcgccga

SEQ ID NO: 72 - DNA, 32 bp, Artificial Sequence
  Description of Artificial Sequence Synthetic oligonucleotide
caatgacactccaaaaggagccttacaacgcc

SEQ ID NO: 73 - DNA, 32 bp, Artificial Sequence
  Description of Artificial Sequence Synthetic oligonucleotide
ccagcaggggcaaaatcccttataaagccggc

SEQ ID NO: 74 - DNA, 32 bp, Artificial Sequence
  Description of Artificial Sequence Synthetic oligonucleotide
gctcacaatgtaaagcctggggtgggtttgcc

SEQ ID NO: 75 - DNA, 32 bp, Artificial Sequence
  Description of Artificial Sequence Synthetic oligonucleotide
gcttctggtcaggctgcgcaactgtgttatcc

SEQ ID NO: 76 - DNA, 32 bp, Artificial Sequence
  Description of Artificial Sequence Synthetic oligonucleotide
gttaaaattttaaccaataggaacccggcacc

SEQ ID NO: 77 - DNA, 32 bp, Artificial Sequence
  Description of Artificial Sequence Synthetic oligonucleotide
aggtaaagaaatcaccatcaatataatatttt

SEQ ID NO: 78 - DNA, 32 bp, Artificial Sequence
  Description of Artificial Sequence Synthetic oligonucleotide
tcgcaaatggggcgcgagctgaaataatgtgt

SEQ ID NO: 79 - DNA, 32 bp, Artificial Sequence
  Description of Artificial Sequence Synthetic oligonucleotide
aagaggaacgagcttcaaagcgaagatacatt

SEQ ID NO: 80 - DNA, 32 bp, Artificial Sequence
  Description of Artificial Sequence Synthetic oligonucleotide
ggaattactcgtttaccagacgacaaaagatt

SEQ ID NO: 81 - DNA, 32 bp, Artificial Sequence
  Description of Artificial Sequence Synthetic oligonucleotide
ccaaatcacttgccctgacgagaacgccaaaa

SEQ ID NO: 82 - DNA, 32 bp, Artificial Sequence
  Description of Artificial Sequence Synthetic oligonucleotide
aaacgaaatgacccccagcgattattcattac

SEQ ID NO: 83 - DNA, 32 bp, Artificial Sequence
  Description of Artificial Sequence Synthetic oligonucleotide
tcggtttagcttgataccgatagtccaaccta

SEQ ID NO: 84 - DNA, 32 bp, Artificial Sequence
  Description of Artificial Sequence Synthetic oligonucleotide
tgagtttcgtcaccagtacaaacttaattgta

SEQ ID NO: 85 - DNA, 32 bp, Artificial Sequence
  Description of Artificial Sequence Synthetic oligonucleotide
gaacgtggcgagaaaggaagggaacaaactat

SEQ ID NO: 86 - DNA, 32 bp, Artificial Sequence
  Description of Artificial Sequence Synthetic oligonucleotide
ccgaaatccgaaaatcctgtttgaagccggaa

SEQ ID NO: 87 - DNA, 32 bp, Artificial Sequence
  Description of Artificial Sequence Synthetic oligonucleotide
gcataaagttccacacaacatacgaagcgcca

SEQ ID NO: 88 - DNA, 32 bp, Artificial Sequence
  Description of Artificial Sequence Synthetic oligonucleotide
ttcgccattgccggaaaccaggcattaaatca

SEQ ID NO: 89 - DNA, 32 bp, Artificial Sequence
  Description of Artificial Sequence Synthetic oligonucleotide
gctcattttcgcattaaatttttgagcttaga

SEQ ID NO: 90 - DNA, 32 bp, Artificial Sequence
  Description of Artificial Sequence Synthetic oligonucleotide
agacagtcattcaaaagggtgagaagctatat

SEQ ID NO: 91 - DNA, 32 bp, Artificial Sequence
  Description of Artificial Sequence Synthetic oligonucleotide
tttcatttggtcaataacctgtttatatcgcg

SEQ ID NO: 92 - DNA, 32 bp, Artificial Sequence
  Description of Artificial Sequence Synthetic oligonucleotide
ttttaattgcccgaaagacttcaaaacactat

SEQ ID NO: 93 - DNA, 32 bp, Artificial Sequence
  Description of Artificial Sequence Synthetic oligonucleotide
cataacccgaggcatagtaagagctttttaag

SEQ ID NO: 94 - DNA, 32 bp, Artificial Sequence
  Description of Artificial Sequence Synthetic oligonucleotide
gaataaggacgtaacaaagctgctctaaaaca

SEQ ID NO: 95 - DNA, 32 bp, Artificial Sequence
  Description of Artificial Sequence Synthetic oligonucleotide
ctcatcttgaggcaaaagaatacagtgaattt

SEQ ID NO: 96 - DNA, 32 bp, Artificial Sequence
  Description of Artificial Sequence Synthetic oligonucleotide
cttaaacatcagcttgctttcgagcgtaacac

SEQ ID NO: 97 - DNA, 32 bp, Artificial Sequence
  Description of Artificial Sequence Synthetic oligonucleotide
acgaaccaaaacatcgccattaaatggtggtt

SEQ ID NO: 98 - DNA, 32 bp, Artificial Sequence
  Description of Artificial Sequence Synthetic oligonucleotide
cgacaactaagtattagactttacaataccga

SEQ ID NO: 99 - DNA, 32 bp, Artificial Sequence
  Description of Artificial Sequence Synthetic oligonucleotide
cttttacacagatgaatatacagtaaacaatt

SEQ ID NO: 100 - DNA, 32 bp, Artificial Sequence
  Description of Artificial Sequence Synthetic oligonucleotide
ttaagacgttgaaaacatagcgataacagtac

SEQ ID NO: 101 - DNA, 32 bp, Artificial Sequence
  Description of Artificial Sequence Synthetic oligonucleotide
gcgttatagaaaaagcctgtttagaaggccgg

SEQ ID NO: 102 - DNA, 32 bp, Artificial Sequence
  Description of Artificial Sequence Synthetic oligonucleotide
atcggctgcgagcatgtagaaacctatcatat

SEQ ID NO: 103 - DNA, 32 bp, Artificial Sequence
  Description of Artificial Sequence Synthetic oligonucleotide
cctaatttacgctaacgagcgtctaatcaata

SEQ ID NO: 104 - DNA, 32 bp, Artificial Sequence
  Description of Artificial Sequence Synthetic oligonucleotide
aaaagtaatatcttaccgaagcccttccagag

SEQ ID NO: 105 - DNA, 32 bp, Artificial Sequence
  Description of Artificial Sequence Synthetic oligonucleotide
ttattcatagggaaggtaaatattcattcagt

SEQ ID NO: 106 - DNA, 32 bp, Artificial Sequence
  Description of Artificial Sequence Synthetic oligonucleotide
gagccgccccaccaccggaaccgcgacggaaa

SEQ ID NO: 107 - DNA, 32 bp, Artificial Sequence
  Description of Artificial Sequence Synthetic oligonucleotide
aatgccccgtaacagtgcccgtatctccctca

SEQ ID NO: 108 - DNA, 32 bp, Artificial Sequence
  Description of Artificial Sequence Synthetic oligonucleotide
caagcccaataggaacccatgtacaaacagtt

SEQ ID NO: 109 - DNA, 32 bp, Artificial Sequence
  Description of Artificial Sequence Synthetic oligonucleotide
cggccttgctggtaatatccagaacgaactga

SEQ ID NO: 110 - DNA, 32 bp, Artificial Sequence
  Description of Artificial Sequence Synthetic oligonucleotide
tagccctaccagcagaagataaaaacatttga

SEQ ID NO: 111 - DNA, 32 bp, Artificial Sequence
  Description of Artificial Sequence Synthetic oligonucleotide
ggatttagcgtattaaatcctttgttttcagg

SEQ ID NO: 112 - DNA, 32 bp, Artificial Sequence
  Description of Artificial Sequence Synthetic oligonucleotide
tttaacgttcgggagaaacaataattttccct

SEQ ID NO: 113 - DNA, 32 bp, Artificial Sequence
  Description of Artificial Sequence Synthetic oligonucleotide
tagaatccctgagaagagtcaataggaatcat

SEQ ID NO: 114 - DNA, 32 bp, Artificial Sequence
  Description of Artificial Sequence Synthetic oligonucleotide
aattactacaaattcttaccagtaatcccatc

SEQ ID NO: 115 - DNA, 32 bp, Artificial Sequence
  Description of Artificial Sequence Synthetic oligonucleotide
ctaatttatctttccttatcattcatcctgaa

SEQ ID NO: 116 - DNA, 32 bp, Artificial Sequence
  Description of Artificial Sequence Synthetic oligonucleotide
tcttaccagccagttacaaaataaatgaaata

SEQ ID NO: 117 - DNA, 32 bp, Artificial Sequence
  Description of Artificial Sequence Synthetic oligonucleotide
gcaatagcgcagatagccgaacaattcaaccg

SEQ ID NO: 118 - DNA, 32 bp, Artificial Sequence
  Description of Artificial Sequence Synthetic oligonucleotide
attgagggtaaaggtgaattatcaatcaccgg

SEQ ID NO: 119 - DNA, 32 bp, Artificial Sequence
  Description of Artificial Sequence Synthetic oligonucleotide
aaccagagaccctcagaaccgccaggggtcag

SEQ ID NO: 120 - DNA, 32 bp, Artificial Sequence
  Description of Artificial Sequence Synthetic oligonucleotide
tgccttgactgcctatttcggaacagggatag

SEQ ID NO: 121 - DNA, 32 bp, Artificial Sequence
  Description of Artificial Sequence Synthetic oligonucleotide
aggcggtcattagtctttaatgcgcaatatta

SEQ ID NO: 122 - DNA, 32 bp, Artificial Sequence
  Description of Artificial Sequence Synthetic oligonucleotide
ttattaatgccgtcaatagataatcagaggtg

SEQ ID NO: 123 - DNA, 32 bp, Artificial Sequence
  Description of Artificial Sequence Synthetic oligonucleotide
cctgattgaaagaaattgcgtagacccgaacg

SEQ ID NO: 124 - DNA, 32 bp, Artificial Sequence
  Description of Artificial Sequence Synthetic oligonucleotide
atcaaaatcgtcgctattaattaacggattcg

SEQ ID NO: 125 - DNA, 32 bp, Artificial Sequence
  Description of Artificial Sequence Synthetic oligonucleotide
acgctcaaaataagaataaacaccgtgaattt

SEQ ID NO: 126 - DNA, 32 bp, Artificial Sequence
  Description of Artificial Sequence Synthetic oligonucleotide
ggtattaagaacaagaaaaataattaaagcca

SEQ ID NO: 127 - DNA, 32 bp, Artificial Sequence
  Description of Artificial Sequence Synthetic oligonucleotide
attatttaacccagctacaattttcaagaacg

SEQ ID NO: 128 - DNA, 32 bp, Artificial Sequence
  Description of Artificial Sequence Synthetic oligonucleotide
gaaggaaaataagagcaagaaacaacagccat

SEQ ID NO: 129 - DNA, 32 bp, Artificial Sequence
  Description of Artificial Sequence Synthetic oligonucleotide
gacttgagagacaaaagggcgacaagttacca

SEQ ID NO: 130 - DNA, 32 bp, Artificial Sequence
  Description of Artificial Sequence Synthetic oligonucleotide
gccaccactcttttcataatcaaaccgtcacc

SEQ ID NO: 131 - DNA, 32 bp, Artificial Sequence
  Description of Artificial Sequence Synthetic oligonucleotide
ctgaaacaggtaataagttttaacccctcaga

SEQ ID NO: 132 - DNA, 32 bp, Artificial Sequence
  Description of Artificial Sequence Synthetic oligonucleotide
ctcagagccaccaccctcattttcctattatt

SEQ ID NO: 133 - DNA, 32 bp, Artificial Sequence
  Description of Artificial Sequence Synthetic oligonucleotide
ccgccagccattgcaacaggaaaaatattttt

SEQ ID NO: 134 - DNA, 32 bp, Artificial Sequence
  Description of Artificial Sequence Synthetic oligonucleotide
gaatggctagtattaacaccgcctcaactaat

SEQ ID NO: 135 - DNA, 32 bp, Artificial Sequence
  Description of Artificial Sequence Synthetic oligonucleotide
agattagatttaaaagtttgagtacacgtaaa

SEQ ID NO: 136 - DNA, 32 bp, Artificial Sequence
  Description of Artificial Sequence Synthetic oligonucleotide
acagaaatctttgaataccaagttccttgctt

SEQ ID NO: 137 - DNA, 32 bp, Artificial Sequence
  Description of Artificial Sequence Synthetic oligonucleotide
ctgtaaatcataggtctgagagacgataaata

SEQ ID NO: 138 - DNA, 32 bp, Artificial Sequence
  Description of Artificial Sequence Synthetic oligonucleotide
aggcgttacagtagggcttaattgacaataga

SEQ ID NO: 139 - DNA, 32 bp, Artificial Sequence
  Description of Artificial Sequence Synthetic oligonucleotide
taagtcctaccaagtaccgcactcttagttgc

SEQ ID NO: 140 - DNA, 32 bp, Artificial Sequence
  Description of Artificial Sequence Synthetic oligonucleotide
tattttgctcccaatccaaataagtgagttaa

SEQ ID NO: 141 - DNA, 32 bp, Artificial Sequence
  Description of Artificial Sequence Synthetic oligonucleotide
gcccaataccgaggaaacgcaataggtttacc

SEQ ID NO: 142 - DNA, 32 bp, Artificial Sequence
  Description of Artificial Sequence Synthetic oligonucleotide
agcgccaaccatttgggaattagattattagc

SEQ ID NO: 143 - DNA, 32 bp, Artificial Sequence
  Description of Artificial Sequence Synthetic oligonucleotide
gtttgccacctcagagccgccaccgatacagg

SEQ ID NO: 144 - DNA, 32 bp, Artificial Sequence
  Description of Artificial Sequence Synthetic oligonucleotide
agtgtacttgaaagtattaagaggccgccacc

SEQ ID NO: 145 - DNA, 32 bp, Artificial Sequence
  Description of Artificial Sequence Synthetic oligonucleotide
gccacgctatacgtggcacagacaacgctcat

SEQ ID NO: 146 - DNA, 32 bp, Artificial Sequence
  Description of Artificial Sequence Synthetic oligonucleotide
attttgcgtctttaggagcactaagcaacagt

SEQ ID NO: 147 - DNA, 32 bp, Artificial Sequence
  Description of Artificial Sequence Synthetic oligonucleotide
gcgcagagatatcaaaattatttgacattatc

SEQ ID NO: 148 - DNA, 32 bp, Artificial Sequence
  Description of Artificial Sequence Synthetic oligonucleotide
taacctccatatgtgagtgaataaacaaaatc

SEQ ID NO: 149 - DNA, 32 bp, Artificial Sequence
  Description of Artificial Sequence Synthetic oligonucleotide
catatttagaaataccgaccgtgttacctttt

SEQ ID NO: 150 - DNA, 32 bp, Artificial Sequence
  Description of Artificial Sequence Synthetic oligonucleotide
caagcaagacgcgcctgtttatcaagaatcgc

SEQ ID NO: 151 - DNA, 32 bp, Artificial Sequence
  Description of Artificial Sequence Synthetic oligonucleotide
ttttgtttaagccttaaatcaagaatcgagaa

SEQ ID NO: 152 - DNA, 32 bp, Artificial Sequence
  Description of Artificial Sequence Synthetic oligonucleotide
atacccaagataacccacaagaataaacgatt

SEQ ID NO: 153 - DNA, 32 bp, Artificial Sequence
  Description of Artificial Sequence Synthetic oligonucleotide
aatcaccaaatagaaaattcatatataacgga

SEQ ID NO: 154 - DNA, 32 bp, Artificial Sequence
  Description of Artificial Sequence Synthetic oligonucleotide
caccagagttcggtcatagcccccgccagcaa

SEQ ID NO: 155 - DNA, 32 bp, Artificial Sequence
  Description of Artificial Sequence Synthetic oligonucleotide
cctcaagaatacatggcttttgatagaaccac

SEQ ID NO: 156 - DNA, 32 bp, Artificial Sequence
  Description of Artificial Sequence Synthetic oligonucleotide
ccctcagaaccgccaccctcagaactgagact

SEQ ID NO: 157 - DNA, 32 bp, Artificial Sequence
  Description of Artificial Sequence Synthetic oligonucleotide
ggaaatacctacattttgacgctcacctgaaa

SEQ ID NO: 158 - DNA, 32 bp, Artificial Sequence
  Description of Artificial Sequence Synthetic oligonucleotide
gcgtaagagagagccagcagcaaaaaggttat

SEQ ID NO: 159 - DNA, 32 bp, Artificial Sequence
  Description of Artificial Sequence Synthetic oligonucleotide
ctaaaatagaacaaagaaaccaccagggttag

SEQ ID NO: 160 - DNA, 32 bp, Artificial Sequence
  Description of Artificial Sequence Synthetic oligonucleotide
aacctaccgcgaattattcatttccagtacat

SEQ ID NO: 161 - DNA, 32 bp, Artificial Sequence
  Description of Artificial Sequence Synthetic oligonucleotide
aaatcaatggcttaggttgggttactaaattt

SEQ ID NO: 162 - DNA, 32 bp, Artificial Sequence
  Description of Artificial Sequence Synthetic oligonucleotide
aatggtttacaacgccaacatgtagttcagct

SEQ ID NO: 163 - DNA, 32 bp, Artificial Sequence
  Description of Artificial Sequence Synthetic oligonucleotide
aatgcagaccgtttttattttcatcttgcggg

SEQ ID NO: 164 - DNA, 32 bp, Artificial Sequence
  Description of Artificial Sequence Synthetic oligonucleotide
aggttttgaacgtcaaaaatgaaagcgctaat

SEQ ID NO: 165 - DNA, 32 bp, Artificial Sequence
  Description of Artificial Sequence Synthetic oligonucleotide
atcagagaaagaactggcatgattttattttg

SEQ ID NO: 166 - DNA, 32 bp, Artificial Sequence
  Description of Artificial Sequence Synthetic oligonucleotide
tcacaatcgtagcaccattaccatcgttttca

SEQ ID NO: 167 - DNA, 32 bp, Artificial Sequence
  Description of Artificial Sequence Synthetic oligonucleotide
tcggcattccgccgccagcattgacgttccag

SEQ ID NO: 168 - DNA, 32 bp, Artificial Sequence
  Description of Artificial Sequence Synthetic oligonucleotide
taagcgtcgaaggattaggattagtaccgcca

SEQ ID NO: 169 - DNA, 32 bp, Artificial Sequence
  Description of Artificial Sequence Synthetic oligonucleotide
ctaaagcaagatagaacccttctgaatcgtct

SEQ ID NO: 170 - DNA, 32 bp, Artificial Sequence
  Description of Artificial Sequence Synthetic oligonucleotide
cggaattattgaaaggaattgaggtgaaaaat

SEQ ID NO: 171 - DNA, 32 bp, Artificial Sequence
  Description of Artificial Sequence Synthetic oligonucleotide
gagcaaaaacttctgaataatggaagaaggag

SEQ ID NO: 172 - DNA, 32 bp, Artificial Sequence
  Description of Artificial Sequence Synthetic oligonucleotide
tatgtaaaccttttttaatggaaaaattacct

SEQ ID NO: 173 - DNA, 32 bp, Artificial Sequence
  Description of Artificial Sequence Synthetic oligonucleotide
agaggcataatttcatcttctgactataacta

SEQ ID NO: 174 - DNA, 32 bp, Artificial Sequence
  Description of Artificial Sequence Synthetic oligonucleotide
tcattacccgacaataaacaacatatttaggc

SEQ ID NO: 175 - DNA, 32 bp, Artificial Sequence
  Description of Artificial Sequence Synthetic oligonucleotide
ctttacagttagcgaacctcccgacgtaggaa

SEQ ID NO: 176 - DNA, 32 bp, Artificial Sequence
  Description of Artificial Sequence Synthetic oligonucleotide
ttattacggtcagagggtaattgaatagcagc

SEQ ID NO: 177 - DNA, 32 bp, Artificial Sequence
  Description of Artificial Sequence Synthetic oligonucleotide
ccggaaacacaccacggaataagtaagactcc

SEQ ID NO: 178 - DNA, 32 bp, Artificial Sequence
  Description of Artificial Sequence Synthetic oligonucleotide
tgaggcaggcgtcagactgtagcgtagcaagg

SEQ ID NO: 179 - DNA, 32 bp, Artificial Sequence
  Description of Artificial Sequence Synthetic oligonucleotide
tgctcagtcagtctctgaatttaccaggaggt

SEQ ID NO: 180 - DNA, 32 bp, Artificial Sequence
  Description of Artificial Sequence Synthetic oligonucleotide
tatcaccgtactcaggaggtttagcggggttt

SEQ ID NO: 181 - DNA, 32 bp, Artificial Sequence
  Description of Artificial Sequence Synthetic oligonucleotide
gaaatggattatttacattggcagacattctg

SEQ ID NO: 182 - DNA, 32 bp, Artificial Sequence
  Description of Artificial Sequence Synthetic oligonucleotide
gccaacagtcaccttgctgaacctgttggcaa

SEQ ID NO: 183 - DNA, 32 bp, Artificial Sequence
  Description of Artificial Sequence Synthetic oligonucleotide
atcaacagtcatcatattcctgattgattgtt

SEQ ID NO: 184 - DNA, 32 bp, Artificial Sequence
  Description of Artificial Sequence Synthetic oligonucleotide
tggattatgaagatgatgaaacaaaatttcat

SEQ ID NO: 185 - DNA, 32 bp, Artificial Sequence
  Description of Artificial Sequence Synthetic oligonucleotide
ttgaattatgctgatgcaaatccacaaatata

SEQ ID NO: 186 - DNA, 32 bp, Artificial Sequence
  Description of Artificial Sequence Synthetic oligonucleotide
ttttagtttttcgagccagtaataaattctgt

SEQ ID NO: 187 - DNA, 32 bp, Artificial Sequence
  Description of Artificial Sequence Synthetic oligonucleotide
ccagacgagcgcccaatagcaagcaagaacgc

SEQ ID NO: 188 - DNA, 32 bp, Artificial Sequence
  Description of Artificial Sequence Synthetic oligonucleotide
gaggcgttagagaataacataaaagaacaccc

SEQ ID NO: 189 - DNA, 32 bp, Artificial Sequence
  Description of Artificial Sequence Synthetic oligonucleotide
tgaacaaacagtatgttagcaaactaaaagaa

SEQ ID NO: 190 - DNA, 32 bp, Artificial Sequence
  Description of Artificial Sequence Synthetic oligonucleotide
acgcaaaggtcaccaatgaaaccaatcaagtt

SEQ ID NO: 191 - DNA, 32 bp, Artificial Sequence
  Description of Artificial Sequence Synthetic oligonucleotide
tgcctttagtcagacgattggcctgccagaat

SEQ ID NO: 192 - DNA, 32 bp, Artificial Sequence
  Description of Artificial Sequence Synthetic oligonucleotide
ggaaagcgaccaggcggataagtgaataggtg

SEQ ID NO: 193 - DNA, 50 bp, Artificial Sequence
  Description of Artificial Sequence Synthetic oligonucleotide
ggtggtggtggttgtggtggtggtggtctaaagttttgtcgtgaattgcg

SEQ ID NO: 194 - DNA, 50 bp, Artificial Sequence
  Description of Artificial Sequence Synthetic oligonucleotide
gagaacctgagtcagtattgcggagatctaaagttttgtcgtgaattgcg

SEQ ID NO: 195 - DNA, 34 bp, Artificial Sequence
  Description of Artificial Sequence Synthetic oligonucleotide
gtaaagcttttttttttttacaaccaccaccacc

SEQ ID NO: 196 - DNA, 34 bp, Artificial Sequence
  Description of Artificial Sequence Synthetic oligonucleotide
gtaaagcaccaccaccaccacaaccaccaccacc

SEQ ID NO: 197 - DNA, 50 bp, Artificial Sequence
  Description of Artificial Sequence Synthetic oligonucleotide
aaaaaaaaaaaaaaaaaaaaaaaaaaactaaagttttgtcgtgaattgcg

SEQ ID NO: 198 - DNA, 34 bp, Artificial Sequence
  Description of Artificial Sequence Synthetic oligonucleotide
gtaaagcttttttttttttttttttttttttttt

SEQ ID NO: 199 - DNA, 50 bp, Artificial Sequence
  Description of Artificial Sequence Synthetic oligonucleotide
ggtggtggtggttgtggtggtggtggtagagcttgacggggaaatcaaaa

SEQ ID NO: 200 - DNA, 34 bp, Artificial Sequence
  Description of Artificial Sequence Synthetic oligonucleotide
tgtagcattttttttttttacaaccaccaccacc

SEQ ID NO: 201 - DNA, 50 bp, Artificial Sequence
  Description of Artificial Sequence Synthetic oligonucleotide
ggtggtggtggttgtggtggtggtggcgagaaaggaagggaacaaactat

SEQ ID NO: 202 - DNA, 34 bp, Artificial Sequence
  Description of Artificial Sequence Synthetic oligonucleotide
tgagtttctttttttttttacaaccaccaccacc

SEQ ID NO: 203 - DNA, 50 bp, Artificial Sequence
  Description of Artificial Sequence Synthetic oligonucleotide
ggtggtggtggttgtggtggtggtggataggaacccatgtacaaacagtt

SEQ ID NO: 204 - DNA, 35 bp, Artificial Sequence
  Description of Artificial Sequence Synthetic oligonucleotide
caagcccattttttttttttacaaccaccaccacc

SEQ ID NO: 205 - DNA, 50 bp, Artificial Sequence
  Description of Artificial Sequence Synthetic oligonucleotide
ggtggtggtggttgtggtggtggtggcaccaccctcattttcctattatt

SEQ ID NO: 206 - DNA, 34 bp, Artificial Sequence
  Description of Artificial Sequence Synthetic oligonucleotide
ccgccagctttttttttttacaaccaccaccacc

SEQ ID NO: 207 - DNA, 50 bp, Artificial Sequence
  Description of Artificial Sequence Synthetic oligonucleotide
ggtggtggtggttgtggtggtggtggctacattttgacgctcacctgaaa

SEQ ID NO: 208 - DNA, 34 bp, Artificial Sequence
  Description of Artificial Sequence Synthetic oligonucleotide
ccctcagttttttttttttacaaccaccaccacc

SEQ ID NO: 209 - DNA, 16 bp, Artificial Sequence
  Description of Artificial Sequence Synthetic oligonucleotide
taaactctttgcgcac

SEQ ID NO: 210 - DNA, 48 bp, Artificial Sequence
  Description of Artificial Sequence Synthetic oligonucleotide
gtgcgcaaagagtttacaaaattaaagtacggtgtctggaagaggtca

SEQ ID NO: 211 - DNA, 48 bp, Artificial Sequence
  Description of Artificial Sequence Synthetic oligonucleotide
gtgcgcaaagagtttatttttgcgcagaaaacgagaatgaatgtttag

SEQ ID NO: 212 - DNA, 48 bp, Artificial Sequence
  Description of Artificial Sequence Synthetic oligonucleotide
gtgcgcaaagagtttacgattttagaggacagatgaacggcgcgacct

SEQ ID NO: 213 - DNA, 48 bp, Artificial Sequence
  Description of Artificial Sequence Synthetic oligonucleotide
gtgcgcaaagagtttagctccatgagaggctttgaggactagggagtt

SEQ ID NO: 214 - DNA, 48 bp, Artificial Sequence
  Description of Artificial Sequence Synthetic oligonucleotide
gtgcgcaaagagtttatccatatacatacaggcaaggcaactttattt

SEQ ID NO: 215 - DNA, 48 bp, Artificial Sequence
  Description of Artificial Sequence Synthetic oligonucleotide
gtgcgcaaagagtttaccaggcgcttaatcattgtgaattacaggtag

SEQ ID NO: 216 - DNA, 48 bp, Artificial Sequence
  Description of Artificial Sequence Synthetic oligonucleotide
gtgcgcaaagagtttacaataaatacagttgattcccaatttagagag

SEQ ID NO: 217 - DNA, 48 bp, Artificial Sequence
  Description of Artificial Sequence Synthetic oligonucleotide
gtgcgcaaagagtttatacctttaaggtctttaccctgacaaagaagt

SEQ ID NO: 218 - DNA, 48 bp, Artificial Sequence
  Description of Artificial Sequence Synthetic oligonucleotide
gtgcgcaaagagtttacgagtagaactaatagtagtagcaaaccctca

SEQ ID NO: 219 - DNA, 48 bp, Artificial Sequence
  Description of Artificial Sequence Synthetic oligonucleotide
gtgcgcaaagagtttatcagaagcctccaacaggtcaggatctgcgaa

SEQ ID NO: 220 - DNA, 48 bp, Artificial Sequence
  Description of Artificial Sequence Synthetic oligonucleotide
gtgcgcaaagagtttaggcgatcgcactccagccagctttgccatcaa

SEQ ID NO: 221 - DNA, 48 bp, Artificial Sequence
  Description of Artificial Sequence Synthetic oligonucleotide
gtgcgcaaagagtttaaaataattttaaattgtaaacgttgatattca

SEQ ID NO: 222 - DNA, 48 bp, Artificial Sequence
  Description of Artificial Sequence Synthetic oligonucleotide
gtgcgcaaagagtttatcaattcttttagtttgaccattaccagaccg

SEQ ID NO: 223 - DNA, 48 bp, Artificial Sequence
  Description of Artificial Sequence Synthetic oligonucleotide
gtgcgcaaagagtttagaagcaaaaaagcggattgcatcagataaaaa

SEQ ID NO: 224 - DNA, 48 bp, Artificial Sequence
  Description of Artificial Sequence Synthetic oligonucleotide
gtgcgcaaagagtttagcttctggtcaggctgcgcaactgtgttatcc

SEQ ID NO: 225 - DNA, 48 bp, Artificial Sequence
  Description of Artificial Sequence Synthetic oligonucleotide
gtgcgcaaagagtttatcgcaaatggggcgcgagctgaaataatgtgt

SEQ ID NO: 226 - DNA, 48 bp, Artificial Sequence
  Description of Artificial Sequence Synthetic oligonucleotide
gtgcgcaaagagtttatttcatttggtcaataacctgtttatatcgcg

SEQ ID NO: 227 - DNA, 48 bp, Artificial Sequence
  Description of Artificial Sequence Synthetic oligonucleotide
gtgcgcaaagagtttattttaattgcccgaaagacttcaaaacactat

SEQ ID NO: 228 - DNA, 48 bp, Artificial Sequence
  Description of Artificial Sequence Synthetic oligonucleotide
gtgcgcaaagagtttaatcggctgcgagcatgtagaaacctatcatat

SEQ ID NO: 229 - DNA, 48 bp, Artificial Sequence
  Description of Artificial Sequence Synthetic oligonucleotide
gtgcgcaaagagtttacctaatttacgctaacgagcgtctaatcaata

SEQ ID NO: 230 - DNA, 48 bp, Artificial Sequence
  Description of Artificial Sequence Synthetic oligonucleotide
gtgcgcaaagagtttactaatttatctttccttatcattcatcctgaa

SEQ ID NO: 231 - DNA, 48 bp, Artificial Sequence
  Description of Artificial Sequence Synthetic oligonucleotide
gtgcgcaaagagtttatcttaccagccagttacaaaataaatgaaata

SEQ ID NO: 232 - DNA, 48 bp, Artificial Sequence
  Description of Artificial Sequence Synthetic oligonucleotide
gtgcgcaaagagtttaattgagggtaaaggtgaattatcaatcaccgg

SEQ ID NO: 233 - DNA, 48 bp, Artificial Sequence
  Description of Artificial Sequence Synthetic oligonucleotide
gtgcgcaaagagtttaaaccagagaccctcagaaccgccaggggtcag

SEQ ID NO: 234 - DNA, 48 bp, Artificial Sequence
  Description of Artificial Sequence Synthetic oligonucleotide
gtgcgcaaagagtttaggtattaagaacaagaaaaataattaaagcca

SEQ ID NO: 235 - DNA, 48 bp, Artificial Sequence
  Description of Artificial Sequence Synthetic oligonucleotide
gtgcgcaaagagtttagacttgagagacaaaagggcgacaagttacca

SEQ ID NO: 236 - DNA, 48 bp, Artificial Sequence
  Description of Artificial Sequence Synthetic oligonucleotide
gtgcgcaaagagtttataagtcctaccaagtaccgcactcttagttgc

SEQ ID NO: 237 - DNA, 48 bp, Artificial Sequence
  Description of Artificial Sequence Synthetic oligonucleotide
gtgcgcaaagagtttatattttgctcccaatccaaataagtgagttaa

SEQ ID NO: 238 - DNA, 48 bp, Artificial Sequence
  Description of Artificial Sequence Synthetic oligonucleotide
gtgcgcaaagagtttacaagcaagacgcgcctgtttatcaagaatcgc

SEQ ID NO: 239 - DNA, 48 bp, Artificial Sequence
  Description of Artificial Sequence Synthetic oligonucleotide
gtgcgcaaagagtttattttgtttaagccttaaatcaagaatcgagaa

SEQ ID NO: 240 - DNA, 48 bp, Artificial Sequence
  Description of Artificial Sequence Synthetic oligonucleotide
gtgcgcaaagagtttaaacctaccgcgaattattcatttccagtacat

SEQ ID NO: 241 - DNA, 48 bp, Artificial Sequence
  Description of Artificial Sequence Synthetic oligonucleotide
gtgcgcaaagagtttaaaatcaatggcttaggttgggttactaaattt

SEQ ID NO: 242 - DNA, 48 bp, Artificial Sequence
  Description of Artificial Sequence Synthetic oligonucleotide
gtgcgcaaagagtttaaatgcagaccgtttttattttcatcttgcggg

SEQ ID NO: 243 - DNA, 48 bp, Artificial Sequence
  Description of Artificial Sequence Synthetic oligonucleotide
gtgcgcaaagagtttaaggttttgaacgtcaaaaatgaaagcgctaat

SEQ ID NO: 244 - DNA, 48 bp, Artificial Sequence
  Description of Artificial Sequence Synthetic oligonucleotide
gtgcgcaaagagtttagagcaaaaacttctgaataatggaagaaggag

SEQ ID NO: 245 - DNA, 48 bp, Artificial Sequence
  Description of Artificial Sequence Synthetic oligonucleotide
gtgcgcaaagagtttatcattacccgacaataaacaacatatttaggc

SEQ ID NO: 246 - DNA, 48 bp, Artificial Sequence
  Description of Artificial Sequence Synthetic oligonucleotide
gtgcgcaaagagtttactttacagttagcgaacctcccgacgtaggaa

SEQ ID NO: 247 - DNA, 47 bp, Artificial Sequence
  Description of Artificial Sequence Synthetic oligonucleotide
aaaaaaaaaaaaaaacaaaaatcattgctccttttgataagtttcat

SEQ ID NO: 248 - DNA, 47 bp, Artificial Sequence
  Description of Artificial Sequence Synthetic oligonucleotide
aaaaaaaaaaaaaaaaaagattcagggggtaatagtaaaccataaat

SEQ ID NO: 249 - DNA, 47 bp, Artificial Sequence
  Description of Artificial Sequence Synthetic oligonucleotide
aaaaaaaaaaaaaaatttgccagatcagttgagatttagtggtttaa

SEQ ID NO: 250 - DNA, 47 bp, Artificial Sequence
  Description of Artificial Sequence Synthetic oligonucleotide
aaaaaaaaaaaaaaagcaaatatcgcgtctggccttcctggcctcag

SEQ ID NO: 251 - DNA, 47 bp, Artificial Sequence
  Description of Artificial Sequence Synthetic oligonucleotide
aaaaaaaaaaaaaaatatattttagctgataaattaatgttgtataa

SEQ ID NO: 252 - DNA, 47 bp, Artificial Sequence
  Description of Artificial Sequence Synthetic oligonucleotide
aaaaaaaaaaaaaaaaccgttctaaatgcaatgcctgagaggtggca

SEQ ID NO: 253 - DNA, 47 bp, Artificial Sequence
  Description of Artificial Sequence Synthetic oligonucleotide
aaaaaaaaaaaaaaaattatttaacccagctacaattttcaagaacg

SEQ ID NO: 254 - DNA, 46 bp, Artificial Sequence
  Description of Artificial Sequence Synthetic oligonucleotide
aaaaaaaaaaaaaagaaggaaaataagagcaagaaacaacagccat

SEQ ID NO: 255 - DNA, 47 bp, Artificial Sequence
  Description of Artificial Sequence Synthetic oligonucleotide
aaaaaaaaaaaaaaagcccaataccgaggaaacgcaataggtttacc

SEQ ID NO: 256 - DNA, 47 bp, Artificial Sequence
  Description of Artificial Sequence Synthetic oligonucleotide
aaaaaaaaaaaaaaataacctccatatgtgagtgaataaacaaaatc

SEQ ID NO: 257 - DNA, 47 bp, Artificial Sequence
  Description of Artificial Sequence Synthetic oligonucleotide
aaaaaaaaaaaaaaacatatttagaaataccgaccgtgttacctttt

SEQ ID NO: 258 - DNA, 47 bp, Artificial Sequence
  Description of Artificial Sequence Synthetic oligonucleotide
aaaaaaaaaaaaaaaaatggtttacaacgccaacatgtagttcagct

SEQ ID NO: 259 - DNA, 54 bp, Artificial Sequence
  Description of Artificial Sequence Synthetic oligonucleotide
ggtggtggtggttgtggtggtggtggttttcgatggcccactacgtaaaccgtc

SEQ ID NO: 260 - DNA, 54 bp, Artificial Sequence
  Description of Artificial Sequence Synthetic oligonucleotide
ggtggtggtggttgtggtggtggtggttttcggtttgcgtattgggaacgcgcg

SEQ ID NO: 261 - DNA, 54 bp, Artificial Sequence
  Description of Artificial Sequence Synthetic oligonucleotide
ggtggtggtggttgtggtggtggtggttttgacagcatcggaacgaaccctcag

SEQ ID NO: 262 - DNA, 54 bp, Artificial Sequence
  Description of Artificial Sequence Synthetic oligonucleotide
ggtggtggtggttgtggtggtggtggattttactttcaacagtttctgggattt

SEQ ID NO: 263 - DNA, 54 bp, Artificial Sequence
  Description of Artificial Sequence Synthetic oligonucleotide
ggtggtggtggttgtggtggtggtggttttaccagtaataaaagggattcacca

SEQ ID NO: 264 - DNA, 54 bp, Artificial Sequence
  Description of Artificial Sequence Synthetic oligonucleotide
ggtggtggtggttgtggtggtggtggttttaatcaatatctggtcacaaatatc

SEQ ID NO: 265 - DNA, 54 bp, Artificial Sequence
  Description of Artificial Sequence Synthetic oligonucleotide
ggtggtggtggttgtggtggtggtggttttataaatcctcattaaatgatattc

SEQ ID NO: 266 - DNA, 54 bp, Artificial Sequence
  Description of Artificial Sequence Synthetic oligonucleotide
ggtggtggtggttgtggtggtggtggtttttataagtatagcccggccgtcgag

SEQ ID NO: 267 - DNA, 47 bp, Artificial Sequence
  Description of Artificial Sequence Synthetic oligonucleotide
aaaaaaaaaaaaaaaatagtaaaccataaatcaaaaatcattgctcc

SEQ ID NO: 268 - DNA, 55 bp, Artificial Sequence
  Description of Artificial Sequence Synthetic oligonucleotide
aaaaaaaaaaaaaaattttgataagtttcattccatatacatacaggcaaggcaa

SEQ ID NO: 269 - DNA, 55 bp, Artificial Sequence
  Description of Artificial Sequence Synthetic oligonucleotide
aaaaaaaaaaaaaaaaccctgacaaagaagttttgccagatcagttgagatttag

SEQ ID NO: 270 - DNA, 47 bp, Artificial Sequence
  Description of Artificial Sequence Synthetic oligonucleotide
aaaaaaaaaaaaaaaaattaatgttgtataagcaaatatcgcgtctg

SEQ ID NO: 271 - DNA, 55 bp, Artificial Sequence
  Description of Artificial Sequence Synthetic oligonucleotide
aaaaaaaaaaaaaaagccttcctggcctcaggaagatcggtgcgggcctcttcgc

SEQ ID NO: 272 - DNA, 55 bp, Artificial Sequence
  Description of Artificial Sequence Synthetic oligonucleotide
aaaaaaaaaaaaaaataaacgttgatattcaaccgttctaaatgcaatgcctgag

SEQ ID NO: 273 - DNA, 47 bp, Artificial Sequence
  Description of Artificial Sequence Synthetic oligonucleotide
aaaaaaaaaaaaaaaaagaaacaacagccatattatttaacccagct

SEQ ID NO: 274 - DNA, 55 bp, Artificial Sequence
  Description of Artificial Sequence Synthetic oligonucleotide
aaaaaaaaaaaaaaaacaattttcaagaacgggtattaagaacaagaaaaataat

SEQ ID NO: 275 - DNA, 55 bp, Artificial Sequence
  Description of Artificial Sequence Synthetic oligonucleotide
aaaaaaaaaaaaaaacaaataagtgagttaagcccaataccgaggaaacgcaata

SEQ ID NO: 276 - DNA, 47 bp, Artificial Sequence
  Description of Artificial Sequence Synthetic oligonucleotide
aaaaaaaaaaaaaaagaccgtgttacctttttaacctccatatgtga

SEQ ID NO: 277 - DNA, 55 bp, Artificial Sequence
  Description of Artificial Sequence Synthetic oligonucleotide
aaaaaaaaaaaaaaagtgaataaacaaaatcgcgcagagatatcaaaattatttg

SEQ ID NO: 278 - DNA, 53 bp, Artificial Sequence
  Description of Artificial Sequence Synthetic oligonucleotide
aaaaaaaaaaaaattgggttactaaatttaatggtttacaacgccaacatgta

SEQ ID NO: 279 - DNA, 26 bp, Artificial Sequence
  Description of Artificial Sequence Synthetic oligonucleotide
ggtggtggtggttgtggtggtggtgg

SEQ ID NO: 280 - DNA, 34 bp, Artificial Sequence
  Description of Artificial Sequence Synthetic oligonucleotide
  modified_base (1)..(8): a, c, t, g, unknown or other
nnnnnnnntttttttttttacaaccaccaccacc

SEQ ID NO: 281 - DNA, 50 bp, Artificial Sequence
  Description of Artificial Sequence Synthetic oligonucleotide
  modified_base (27)..(50): a, c, t, g, unknown or other
ggtggtggtggttgtggtggtggtggnnnnnnnnnnnnnnnnnnnnnnnn

SEQ ID NO: 282 - DNA, 34 bp, Artificial Sequence
  Description of Artificial Sequence Synthetic oligonucleotide
  modified_base (1)..(8): a, c, t, g, unknown or other
nnnnnnnnccaccaccaccacaaccaccaccacc

(19) United States
(12) Patent Application Publication
(10) Pub. No.: US 2019/0240248 A1
(43) Pub. Date: Aug. 8, 2019

(54) DNA NANOROBOT AND METHODS OF USE THEREOF

(71) Applicants: ARIZONA BOARD OF REGENTS ON BEHALF OF ARIZONA STATE UNIVERSITY, Scottsdale, AZ (US); NATIONAL CENTER FOR NANOSCIENCE AND TECHNOLOGY, Beijing (CN)

(72) Inventors: Hao YAN, Chandler, AZ (US); Qiao JIANG, Beijing (CN); Suping LI, Beijing (CN); Baoquan DING, Beijing (CN); Guangjun NIE, Beijing (CN)

(21) Appl. No.: 16/208,103

(22) Filed: Dec. 3, 2018

Related U.S. Application Data
(60) Provisional application No. 62/616,131, filed on Jan. 11, 2018.

(30) Foreign Application Priority Data
Dec. 7, 2017 (CN) PCT/CN2017/115004
Sep. 20, 2018 (CN) PCT/CN2018/106742

(51) Int. Cl. A61K 31/7088; A61K 9/00; A61K 47/26; C12N 7/00; A61K 9/51; C12N 15/115; A61P 35/00

(52) U.S. Cl. CPC A61K 31/7088; A61K 9/0019; A61K 47/26; C12N 7/00; A61K 9/51; B82Y 5/00; A61P 35/00; C12N 2795/00022; C12N 2310/16; C12N 2795/00032; C12N 2795/00042; C12N 15/115

(57) ABSTRACT

In certain embodiments, the present invention provides a DNA nanostructure nanorobot comprising: a single stranded DNA scaffold strand of about 5,000 to 10,000 bases in length; a plurality of staple strands of DNA, wherein each staple strands are about 20 to 40 bases in length, wherein each staple strand has a unique sequence and is hybridized to a specific position on the DNA scaffold strand, wherein the plurality of staple strands hybridized to the DNA scaffold form a sheet having a top surface and a bottom surface; and one or more fastener strands of DNA, wherein the one or more fastener strands of DNA is capable of fastening the sheet into an origami structure.

### RELATED APPLICATIONS

This application claims priority to International Application Number PCT/CN2017/115004, filed Dec. 7, 2017 and International Application Number PCT/CN2018/106742, filed Sep. 20, 2018; this application also claims priority to U.S. Provisional Application No. 62/616,131, filed Jan. 11, 2018. The entire content of the applications referenced above are hereby incorporated by reference herein.

### STATEMENT REGARDING FEDERALLY SPONSORED RESEARCH

This invention was made with government support under R01 GM104960 awarded by the National Institutes of Health. The government has certain rights in the invention.

### BACKGROUND

DNA molecules have been shown to be excellent platforms for the design and construction of mechanical molecular devices that sense, actuate and exert critical functions when exposed to external signals¹. DNA-based robotics have been utilized as imaging probes²⁻⁴ and cargo delivery vehicles⁴⁻⁶ in cultured cells^{2, 5}, multicellular organisms⁴ and insects⁷. However, robotic DNA machines serving as intelligent vehicles for in vivo targeting drug delivery and controlled release in mammals have not yet been described. In contrast to the strategies of killing tumor cells directly by cytotoxic anticancer drugs or by anti-angiogenic agents⁸, selective occlusion of tumor blood vessels, to deprive tumors of nutrients and oxygen and start an avalanche of tumor cell death, is an attractive strategy for combating cancer⁹⁻¹². Vascular occlusion can exert its effects within hours following the rapid induction of thrombus formation in tumor vessels. This leads to much shorter treatment duration than many other therapies, and carries a decreased risk of resistance development. Moreover, vascular occlusion in tumors is a strategy that can be used for many types of cancer, since all solid tumor-feeding vessels are essentially the same. The coagulation protease, thrombin, regulates platelet aggregation by activating platelets and converting circulating fibrinogen to fibrin¹³, ultimately leading to obstructive thrombosis. Naked thrombin is short lived in the circulation and induces coagulation events indiscriminately, and thus has never been used as an injectable therapeutic vessel occluding agent in cancer treatment. A critical challenge for introducing thrombin as a potent antitumor therapeutic is the precise delivery of sufficient quantities of the active protease solely to tumor sites in a highly controlled manner to minimize its effects in healthy tissues.

Accordingly, safe and effective compositions and methods are needed to treat tumors.

### SUMMARY

As described herein, a DNA nanostructure nanorobot was constructed and its function as a molecular payload carrier has been demonstrated.

Certain embodiments of the invention provide a DNA nanostructure nanorobot comprising:

a single stranded DNA scaffold strand of about 5000 to 10,000 bases in length;

a plurality of staple strands of DNA, wherein each staple strand is about 20 to 40 bases in length, wherein each staple strand has a unique sequence and is hybridized to a specific position on the DNA scaffold strand, wherein the plurality of staple strands hybridized to the DNA scaffold form a sheet having a top surface and a bottom surface;

one or more fastener strands of DNA, wherein the one or more fastener strands of DNA is capable of fastening the sheet into an origami structure. As used herein, the term “origami structure” means a 3-dimentional structure. As used herein, a “fastener strand” is an oligonucleotide that operably links two strands of DNA to form an origami structure/shape. For example, a plurality of fastener strands can bind (“tie”) two edges of a rectangular DNA origami sheet to form a tube shape.

Certain embodiments of the invention provide DNA nanostructure nanorobot comprising:

a single stranded DNA scaffold strand comprising M13 phage DNA;

a plurality of staple strands 13-204 (as described herein) of DNA wherein the plurality of staple strands hybridized to the DNA scaffold to forms a rectangular sheet having a top surface, a bottom surface, and four corners;

at least six fastener strands of DNA, wherein each fastener strand of DNA is capable of fastening the rectangular sheet into a tube-shaped origami structure;

four DNA capture strands, wherein each capture strand is operably linked to a thrombin; and

at least four targeting strands, wherein each targeting strand is operably linked to an aptamer specific for nucleolin.

In certain embodimetns, the staple strands are selected from the following Staple strands pool (5′-3′):

```
  13 TGGTTTTTAACGTCAAAGGGCGAAGAACCATC
  14 CTTGCATGCATTAATGAATCGGCCCGCCAGGG
  15 TAGATGGGGGGTAACGCCAGGGTTGTGCCAAG
  16 CATGTCAAGATTCTCCGTGGGAACCGTTGGTG
  17 CTGTAATATTGCCTGAGAGTCTGGAAAACTAG
  18 TGCAACTAAGCAATAAAGCCTCAGTTATGACC
  19 AAACAGTTGATGGCTTAGAGCTTATTTAAATA
  20 ACGAACTAGCGTCCAATACTGCGGAATGCTTT
  21 CTTTGAAAAGAACTGGCTCATTATTTAATAAA
  22 ACGGCTACTTACTTAGCCGGAACGCTGACCAA
  23 GAGAATAGCTTTTGCGGGATCGTCGGGTAGCA
  24 ACGTTAGTAAATGAATTTTCTGTAAGCGGAGT
  25 ACCCAAATCAAGTTTTTTGGGGTCAAAGAACG
  26 TGGACTCCCTTTTCACCAGTGAGACCTGTCGT
  27 GCCAGCTGCCTGCAGGTCGACTCTGCAAGGCG
  28 ATTAAGTTCGCATCGTAACCGTGCGAGTAACA
  29 ACCCGTCGTCATATGTACCCCGGTAAAGGCTA
  30 TCAGGTCACTTTTGCGGGAGAAGCAGAATTAG
  31 CAAAATTAAAGTACGGTGTCTGGAAGAGGTCA
  32 TTTTTGCGCAGAAAACGAGAATGAATGTTTAG
  33 ACTGGATAACGGAACAACATTATTACCTTATG
  34 CGATTTTAGAGGACAG ATGAACGGCGCGACCT
  35 GCTCCATGAGAGGCTT TGAGGACTAGGGAGTT
  36 AAAGGCCGAAAGGAACAACTAAAGCTTTCCAG
  37 AGCTGATTACAAGAGTCCACTATTGAGGTGCC
  38 CCCGGGTACTTTCCAGTCGGGAAACGGGCAAC
  39 GTTTGAGGGAAAGGGGGATGTGCTAGAGGATC
  40 AGAAAAGCAACATTAAATGTGAGCATCTGCCA
  41 CAACGCAATTTTTGAGAGATCTACTGATAATC
  42 TCCATATACATACAGGCAAGGCAACTTTATTT
  43 CAAAAATCATTGCTCCTTTTGATAAGTTTCAT
  44 AAAGATTCAGGGGGTAATAGTAAACCATAAAT
  45 CCAGGCGCTTAATCATTGTGAATTACAGGTAG
  46 TTTCATGAAAATTGTGTCGAAATCTGTACAGA
  47 AATAATAAGGTCGCTGAGGCTTGCAAAGACTT
  48 CGTAACGATCTAAAGTTTTGTCGTGAATTGCG
  49 GTAAAGCACTAAATCGGAACCCTAGTTGTTCC
  50 AGTTTGGAGCCCTTCACCGCCTGGTTGCGCTC
  51 ACTGCCCGCCGAGCTCGAATTCGTTATTACGC
  52 CAGCTGGCGGACGACGACAGTATCGTAGCCAG
  53 CTTTCATCCCCAAAAACAGGAAGACCGGAGAG
  54 GGTAGCTAGGATAAAAATTTTTAGTTAACATC
  55 CAATAAATACAGTTGATTCCCAATTTAGAGAG
  56 TACCTTTAAGGTCTTTACCCTGACAAAGAAGT
  57 TTTGCCAGATCAGTTGAGATTTAGTGGTTTAA
  58 TTTCAACTATAGGCTGGCTGACCTTGTATCAT
  59 CGCCTGATGGAAGTTTCCATTAAACATAACCG
  60 ATATATTCTTTTTTCACGTTGAAAATAGTTAG
  61 GAGTTGCACGAGATAGGGTTGAGTAAGGGAGC
  62 TCATAGCTACTCACATTAATTGCGCCCTGAGA
  63 GAAGATCGGTGCGGGCCTCTTCGCAATCATGG
  64 GCAAATATCGCGTCTGGCCTTCCTGGCCTCAG
  65 TATATTTTAGCTGATAAATTAATGTTGTATAA
  66 CGAGTAGAACTAATAGTAGTAGCAAACCCTCA
  67 TCAGAAGCCTCCAACAGGTCAGGATCTGCGAA
  68 CATTCAACGCGAGAGGCTTTTGCATATTATAG
  69 AGTAATCTTAAATTGGGCTTGAGAGAATACCA
  70 ATACGTAAAAGTACAACGGAGATTTCATCAAG
  71 AAAAAAGGACAACCATCGCCCACGCGGGTAAA
  72 TGTAGCATTCCACAGACAGCCCTCATCTCCAA
  73 CCCCGATTTAGAGCTTGACGGGGAAATCAAAA
  74 GAATAGCCGCAAGCGGTCCACGCTCCTAATGA
  75 GTGAGCTAGTTTCCTGTGTGAAATTTGGGAAG
  76 GGCGATCGCACTCCAGCCAGCTTTGCCATCAA
  77 AAATAATTTTAAATTGTAAACGTTGATATTCA
  78 ACCGTTCTAAATGCAATGCCTGAGAGGTGGCA
  79 TCAATTCTTTTAGTTTGACCATTACCAGACCG
  80 GAAGCAAAAAAGCGGATTGCATCAGATAAAAA
  81 CCAAAATATAATGCAGATACATAAACACCAGA
  82 ACGAGTAGTGACAAGAACCGGATATACCAAGC
  83 GCGAAACATGCCACTACGAAGGCATGCGCCGA
  84 CAATGACACTCCAAAAGGAGCCTTACAACGCC
  85 CCAGCAGGGGCAAAATCCCTTATAAAGCCGGC
  86 GCTCACAATGTAAAGCCTGGGGTGGGTTTGCC
  87 GCTTCTGGTCAGGCTGCGCAACTGTGTTATCC
  88 GTTAAAATTTTAACCAATAGGAACCCGGCACC
  89 AGGTAAAGAAATCACCATCAATATAATATTTT
  90 TCGCAAATGGGGCGCGAGCTGAAATAATGTGT
  91 AAGAGGAACGAGCTTCAAAGCGAAGATACATT
  92 GGAATTACTCGTTTACCAGACGACAAAAGATT
  93 CCAAATCACTTGCCCTGACGAGAACGCCAAAA
  94 AAACGAAATGACCCCCAGCGATTATTCATTAC
  95 TCGGTTTAGCTTGATACCGATAGTCCAACCTA
  96 TGAGTTTCGTCACCAGTACAAACTTAATTGTA
  97 GAACGTGGCGAGAAAGGAAGGGAACAAACTAT
  98 CCGAAATCCGAAAATCCTGTTTGAAGCCGGAA
  99 GCATAAAGTTCCACACAACATACGAAGCGCCA
100 TTCGCCATTGCCGGAAACCAGGCATTAAATCA
101 GCTCATTTTCGCATTAAATTTTTGAGCTTAGA
102 AGACAGTCATTCAAAAGGGTGAGAAGCTATAT
103 TTTCATTTGGTCAATAACCTGTTTATATCGCG
104 TTTTAATTGCCCGAAAGACTTCAAAACACTAT
105 CATAACCCGAGGCATAGTAAGAGCTTTTTAAG
106 GAATAAGGACGTAACAAAGCTGCTCTAAAACA
107 CTCATCTTGAGGCAAAAGAATACAGTGAATTT
108 CTTAAACATCAGCTTGCTTTCGAGCGTAACAC
109 ACGAACCAAAACATCGCCATTAAATGGTGGTT
110 CGACAACTAAGTATTAGACTTTACAATACCGA
111 CTTTTACACAGATGAATATACAGTAAACAATT
112 TTAAGACGTTGAAAACATAGCGATAACAGTAC
113 GCGTTATAGAAAAAGCCTGTTTAGAAGGCCGG
114 ATCGGCTGCGAGCATGTAGAAACCTATCATAT
115 CCTAATTTACGCTAACGAGCGTCTAATCAATA
116 AAAAGTAATATCTTACCGAAGCCCTTCCAGAG
117 TTATTCATAGGGAAGGTAAATATTCATTCAGT
118 GAGCCGCCCCACCACCGGAACCGCGACGGAAA
119 AATGCCCCGTAACAGTGCCCGTATCTCCCTCA
120 CAAGCCCAATAGGAACCCATGTACAAACAGTT
121 CGGCCTTGCTGGTAATATCCAGAACGAACTGA
122 TAGCCCTACCAGCAGAAGATAAAAACATTTGA
123 GGATTTAGCGTATTAAATCCTTTGTTTTCAGG
124 TTTAACGTTCGGGAGAAACAATAATTTTCCCT
125 TAGAATCCCTGAGAAGAGTCAATAGGAATCAT
126 AATTACTACAAATTCTTACCAGTAATCCCATC
127 CTAATTTATCTTTCCTTATCATTCATCCTGAA
128 TCTTACCAGCCAGTTACAAAATAAATGAAATA
129 GCAATAGCGCAGATAGCCGAACAATTCAACCG
130 ATTGAGGGTAAAGGTGAATTATCAATCACCGG
128 AACCAGAGACCCTCAGAACCGCCAGGGGTCAG
132 TGCCTTGACTGCCTATTTCGGAACAGGGATAG
133 AGGCGGTCATTAGTCTTTAATGCGCAATATTA
134 TTATTAATGCCGTCAATAGATAATCAGAGGTG
135 CCTGATTGAAAGAAATTGCGTAGACCCGAACG
136 ATCAAAATCGTCGCTATTAATTAACGGATTCG
137 ACGCTCAAAATAAGAATAAACACCGTGAATTT
138 GGTATTAAGAACAAGAAAAATAATTAAAGCCA
139 ATTATTTAACCCAGCTACAATTTTCAAGAACG
140 GAAGGAAAATAAGAGCAAGAAACAACAGCCAT
141 GACTTGAGAGACAAAAGGGCGACAAGTTACCA
142 GCCACCACTCTTTTCATAATCAAACCGTCACC
143 CTGAAACAGGTAATAAGTTTTAACCCCTCAGA
144 CTCAGAGCCACCACCCTCATTTTCCTATTATT
145 CCGCCAGCCATTGCAACAGGAAAAATATTTTT
146 GAATGGCTAGTATTAACACCGCCTCAACTAAT
147 AGATTAGATTTAAAAGTTTGAGTACACGTAAA
148 ACAGAAATCTTTGAATACCAAGTTCCTTGCTT
149 CTGTAAATCATAGGTCTGAGAGACGATAAATA
150 AGGCGTTACAGTAGGGCTTAATTGACAATAGA
151 TAAGTCCTACCAAGTACCGCACTCTTAGTTGC
152 TATTTTGCTCCCAATCCAAATAAGTGAGTTAA
153 GCCCAATACCGAGGAAACGCAATAGGTTTACC
154 AGCGCCAACCATTTGGGAATTAGATTATTAGC
155 GTTTGCCACCTCAGAGCCGCCACCGATACAGG
156 AGTGTACTTGAAAGTATTAAGAGGCCGCCACC
157 GCCACGCTATACGTGGCACAGACAACGCTCAT
158 ATTTTGCGTCTTTAGGAGCACTAAGCAACAGT
159 GCGCAGAGATATCAAAATTATTTGACATTATC
160 TAACCTCCATATGTGAGTGAATAAACAAAATC
161 CATATTTAGAAATACCGACCGTGTTACCTTTT
162 CAAGCAAGACGCGCCTGTTTATCAAGAATCGC
163 TTTTGTTTAAGCCTTAAATCAAGAATCGAGAA
164 ATACCCAAGATAACCCACAAGAATAAACGATT
165 AATCACCAAATAGAAAATTCATATATAACGGA
166 CACCAGAGTTCGGTCATAGCCCCCGCCAGCAA
167 CCTCAAGAATACATGGCTTTTGATAGAACCAC
168 CCCTCAGAACCGCCACCCTCAGAACTGAGACT
169 GGAAATACCTACATTTTGACGCTCACCTGAAA
170 GCGTAAGAGAGAGCCAGCAGCAAAAAGGTTAT
171 CTAAAATAGAACAAAGAAACCACCAGGGTTAG
172 AACCTACCGCGAATTATTCATTTCCAGTACAT
173 AAATCAATGGCTTAGGTTGGGTTACTAAATTT
174 AATGGTTTACAACGCCAACATGTAGTTCAGCT
175 AATGCAGACCGTTTTTATTTTCATCTTGCGGG
176 AGGTTTTGAACGTCAAAAATGAAAGCGCTAAT
177 ATCAGAGAAAGAACTGGCATGATTTTATTTTG
178 TCACAATCGTAGCACCATTACCATCGTTTTCA
179 TCGGCATTCCGCCGCCAGCATTGACGTTCCAG
180 TAAGCGTCGAAGGATTAGGATTAGTACCGCCA
181 CTAAAGCAAGATAGAACCCTTCTGAATCGTCT
182 CGGAATTATTGAAAGGAATTGAGGTGAAAAAT
183 GAGCAAAAACTTCTGAATAATGGAAGAAGGAG
184 TATGTAAACCTTTTTTAATGGAAAAATTACCT
185 AGAGGCATAATTTCATCTTCTGACTATAACTA
186 TCATTACCCGACAATAAACAACATATTTAGGC
187 CTTTACAGTTAGCGAACCTCCCGACGTAGGAA
188 TTATTACGGTCAGAGGGTAATTGAATAGCAGC
189 CCGGAAACACACCACGGAATAAGTAAGACTCC
190 TGAGGCAGGCGTCAGACTGTAGCGTAGCAAGG
191 TGCTCAGTCAGTCTCTGAATTTACCAGGAGGT
192 TATCACCGTACTCAGGAGGTTTAGCGGGGTTT
193 GAAATGGATTATTTACATTGGCAGACATTCTG
194 GCCAACAGTCACCTTGCTGAACCTGTTGGCAA
195 ATCAACAGTCATCATATTCCTGATTGATTGTT
196 TGGATTATGAAGATGATGAAACAAAATTTCAT
197 TTGAATTATGCTGATGCAAATCCACAAATATA
198 TTTTAGTTTTTCGAGCCAGTAATAAATTCTGT
199 CCAGACGAGCGCCCAATAGCAAGCAAGAACGC
200 GAGGCGTTAGAGAATAACATAAAAGAACACCC
201 TGAACAAACAGTATGTTAGCAAACTAAAAGAA
202 ACGCAAAGGTCACCAATGAAACCAATCAAGTT
203 TGCCTTTAGTCAGACGATTGGCCTGCCAGAAT
204 GGAAAGCGACCAGGCGGATAAGTGAATAGGTG
```

In certain embodiments, the plurality of imaging strands comprise extended ssDNA sequences that hybridized to fluorescent dye-labeled ssDNA.

Certain embodiments of the invention provide a pharmaceutical composition comprising the DNA nanostructure nanorobot described herein.

Certain embodiments of the invention provide a method of treating a disease or disorder in a subject, comprising administering to the subject a therapeutically effective amount of the DNA nanostructure nanorobot or pharmaceutical composition as described herein.

Certain embodiments of the invention provide a method of inhibiting tumor growth in a subject, comprising administering to the subject a therapeutically effective amount of the DNA nanostructure nanorobot or pharmaceutical composition as described herein.

Certain embodiments of the invention provide a use of the DNA nanostructure nanorobot or a composition as described herein for the manufacture of a medicament for inducing a tumor necrosis response in a subject (e.g., a mammal, such as a human).

Certain embodiments of the invention provide the DNA nanostructure nanorobot or a composition as described herein for the prophylactic or therapeutic treatment a disease or disorder.

Certain embodiments of the invention provide a kit comprising the DNA nanostructure nanorobot or a composition as described herein and instructions for administering the DNA nanostructure nanorobot/composition to a subject to induce an immune response or to treat a disease or disorder.

The invention also provides processes disclosed herein that are useful for preparing a DNA nanostructure nanorobot described herein.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1B. Design and characterization of thrombin-functionalized DNA nanorobot. (FIG. 1a) Schematic illustration of the construction of thrombin-loaded nanorobot by DNA origami, and its reconfiguration into a rectangular DNA sheet in response to nucleolin binding. (I) Single stranded M13 phage genomic DNA is linked by predesigned staple strands, leading to the formation of a rectangular DNA sheet. (II) Thrombin is loaded onto the surface of the DNA sheet structure by hybridization of polyT oligonucleotides conjugated to thrombin molecules with polyA sequences that extend from the surface of the DNA sheet. (III) Addition of the fasteners and targeting strands results in the formation of thrombin-loaded, tubular DNA nanorobots with additional targeting aptamers at both ends. (IV) The tube nanocarrier opens in response to the presence of nucleolin to expose the encapsulated thrombin. (FIG. 1b) DNA nanorobots were examined by AFM and representative images of closed (left) and opened states (right) are shown. The four bright spots displayed on the surface of the origami sheet represent the thrombin molecules (thrombin molecules on the four DNA sheet-thrombin assemblies on the right are highlighted by white circles). Scale bars, 100 nm.

FIGS. 2A-2G. Analysis of DNA nanorobot-triggered activation and endothelial cell targeting. (FIG. 2a) Scheme of Y-shaped fastener strands and dissociation in response to nucleolin recognition. F and Q represent fluorescent and quencher molecules, respectively. The 15-bp partially complementary duplex switches to the G-quadruplex state to form an AS1411-nucleolin complex in response to the nucleolin target protein. (FIG. 2b) Flow cytometry histograms showing Y-shaped fastener dissociation after a 2 h incubation with HUVECs, as measured by cell labeling with FITC-labeled F50 containing AS1411 sequences. HUVECs treated with fasteners of partially (15-bp, red line) and fully complementary (26-bp, green line) AS1411 duplex are shown. Cells incubated with control Y-shapes without the AS1411 portion are represented by the purple line while unstained cells are the black line. (FIG. 2c) Scheme of fluorophore-quencher pair-fastened DNA nanorobot and reconfiguration in response to nucleolin recognition. (FIG. 2d) Flow cytometry histograms showing fastened DNA nanorobot triggered reconfiguration by HUVECs after a 2 h incubation, resulting in enhanced fluorescence intensity. The starved HUVECs, a state in which surface nucleolin expression is down-regulated, cannot open the DNA nanorobot. (FIG. 2e) Schematic representation of the mechanism of action of nanorobot-Th in plasma in the presence of vascular endothelial cells. Thrombin molecules are arranged at four designated locations inside the nanorobot in an inactive state. Closed nanorobot is mixed with mouse plasma and vascular endothelial cells (i.e., HUVECs) to mimic the tumor-associated microenvironment. The nanorobot binds to HUVECs by recognizing the cell surface target protein, nucleolin, and the tube subsequently opens to expose the encapsulated thrombin. Thrombin induces a localized thrombosis by activating platelets and inducing fibrin generation. (FIG. 2f) Induction of coagulation by cell-bound nanorobot loaded with thrombin. HUVECs (10⁵ cells, 100 μl) were incubated with nanorobot and various controls for 1 h at 37° C. Mouse plasma prepared from blood collected with a 3.8% anticoagulant sodium citrate solution (citrate/blood: 1/9, v/v) was added to the cells, and the time until the first fibrin strands formed was recorded. Error bars represent the mean±s.d. of three independent experiments. (FIG. 2g) Confocal microscopy showing the targeting of nanorobot to nucleolin-positive HUVECs. Alexa 594-labeled nanorobot (red) with additional targeting aptamer strands at both ends binds to the cells in the absence of an antibody to nucleolin, while those lacking targeting groups do not bind. The peri-nuclear accumulation of red fluorescence after 8 h incubation indicated internalization of the nanostructures. Images were acquired at 1, 3, 6 or 8 h after incubation of the cells with nanorobot or nanotubes. Nucleus and plasma membrane were stained with DAPI (blue) and DiO (green), respectively. The colocalization of nanostructures with cell membranes appears in yellow (merge). The fluorescence images are representative of three independent experiments and additional high magnification images were shown for the nanorobot-treated cells. Scale bars, 20 μm.

FIGS. 3A-3J. DNA nanorobots targets tumors, induce thrombosis in tumor vessels and inhibit tumor growth in vivo. (FIG. 3a) Optical imaging of a MDA-MB-231 human breast tumor-bearing mouse before and after intravenous injection of Cy5.5-labeled nanorobot. A high-intensity fluorescent signal was detected only in the tumor region of mice 8 h post-injection. 0 h=before injection. (FIG. 3b) In vivo fluorescence intensity at the tumor sites was quantified as Total Fluorescence Intensity (TFI) at the indicated time points after administration of the nanorobots; n=3. (FIG. 3c) FITC-labeled nanorobots were injected intravenously into mice bearing MDA-MB-231 tumors. Tumors were harvested 8 h later, and tumor sections were stained with an anti-CD34 antibody and examined by confocal microscopy. The nanorobot (green) appears in the blood vessel-rich regions (anti-CD34; brown). Nuclei are indicated in blue. Scale bars, 20 μm. (FIG. 3d) Schematic representation of the therapeutic mechanism of nanorobot-Th within tumor vessels. DNA nanorobot-Th was administrated to breast tumor xenografted mice by tail vein injection and targeted tumor-associated vessels to deliver thrombin. The nanorobot-Th binds to the vascular endothelium by recognizing nucleolin and opens to expose the encapsulated thrombin, which induces localized thromboses, tumor infarction and cell necrosis. (FIG. 3e) MDA-MB-231 tumors harvested before and 24, 48 or 72 h post-administration of nanorobot-Th were immunostained for CD41 (activated platelets) to detect thrombosis (brown, red arrows). Scale bars, 50 μm. (FIG. 3f) Tumors harvested before and 24, 48 or 72 h after treatment with nanorobot-Th were stained with haematoxylin and eosin (H&E). Necrotic tissues are denoted by N. Scale bars, 200 μm. Data are representative of three independent experiments. (FIGS. 3g-i) MDA-MB-231 tumor-bearing mice were treated on day 0 with saline, free thrombin, targeted empty nanorobot, nontargeted nanotube-Th, targeted nanotube-Th or nanorobot-Th. Black arrows in g and j highlight the injection time points. Tumor volumes up to day 21 (FIG. 3g) were compared using a Kruskal-Wallis test followed by a Mann-Whitney test. Representative pictures of the tumors (FIG. 3h) were shown and average tumor weights (i) of the indicated groups of mice (n=8). (FIG. 3j) Cumulative survival of MDA-MB-231 tumor-bearing mice (n=10) compared using Kaplan Meier analysis followed by the Log Rank test. Treatment started when the tumor volume reached ˜100 mm³. **p<0.01 and ***p<0.001.

FIG. 4 depicts an unfastened rectangular DNA origami structure having fasteners extending from the edges that can be joined, aptamer-containing targeting strands and drug capture strand operably linked to a therapeutic agent.

FIG. 5 depicts an aptamer-containing targeting strand containing an aptamer portion and an attaching DNA strand.

FIG. 6 depicts an aptamer-containing targeting strand containing an aptamer portion and an attaching DNA strand, and having a quencher moiety attached to one arm of the Y-structure and a fluorophore moiety attached to the second arm of the Y-structure.

FIG. 7 depicts a drug-DNA conjugate capture strand having a ssDNA attachment strand and a drug payload.

FIG. 8 depicts a drug-DNA conjugate capture strand having a ssDNA attachment strand and a drug payload, where the drug payload is operably linked to an imaging agent.

FIG. 9 depicts a drug-DNA conjugate capture strand having a ssDNA attachment strand that is linked to a drug payload by means of a linker.

FIG. 10 depicts a drug-DNA conjugate capture strand having a ssDNA attachment strand that is linked to a drug payload by means of a linker, where the drug payload is operably linked to an imaging agent.

FIG. 11 depicts an unfastened rectangular DNA origami structure having four drug-DNA conjugates operably linked to the origami structure. The drug-DNA conjugates can be attached to either the “top” or the “bottom” (or both) of the origami structure, such that when the origami structure is rolled into a tube, the drug-DNA conjugates can be designed to be either on the inside or outside of the tube.

FIG. 12 depicts an unfastened rectangular DNA origami structure having three drug-DNA conjugates operably linked to the origami structure. The drug-DNA conjugates can be attached to either the “top” or the “bottom” (or both) of the origami structure, such that when the origami structure is rolled into a tube, the drug-DNA conjugates can be designed to be either on the inside or outside of the tube.

FIG. 13 depicts an unfastened rectangular DNA origami structure having two drug-DNA conjugates operably linked to the origami structure. The drug-DNA conjugates can be attached to either the “top” or the “bottom” (or both) of the origami structure, such that when the origami structure is rolled into a tube, the drug-DNA conjugates can be designed to be either on the inside or outside of the tube.

FIG. 14 depicts an unfastened rectangular DNA origami structure having one drug-DNA conjugates operably linked to the origami structure. The drug-DNA conjugate can be attached to either the “top” or the “bottom” of the origami structure, such that when the origami structure is rolled into a tube, the drug-DNA conjugate can be designed to be either on the inside or outside of the tube.

FIG. 15 depicts an exploded view of the DNA origami structure, detailing the hybridization of a single stranded DNA scaffold strand and staple strands, and the interaction of the two staple strands.

FIG. 16 depicts a tube-shaped DNA origami structure having drug-DNA conjugates positioned on the outside of the tube-shaped DNA origami structure.

FIG. 17 depicts a tube-shaped DNA origami structure having drug-DNA conjugates positioned on the inside of the tube-shaped DNA origami structure.

FIG. 18 depicts a tube-shaped DNA origami structure having drug-DNA conjugates positioned on the inside of the tube-shaped DNA origami structure, having aptamer-containing targeting strands positioned at the ends of the tube, and illustrating the fasteners joining the edges of the DNA origami structure so as to form a tube shape.

FIG. 19. The design of the rectangular DNA origami structure blank template with M13 phage single stranded DNA (black) and staple strands (cyan). Each staple strand has its individual sequence and position. To avoid a stacking effect during assembly, twenty-four staple strands along the two wide sides were removed from the pool (1-12 and 205-216).

FIG. 20. DNA origami design with functional strands for thrombin loading and for in vitro and in vivo imaging. For thrombin loading on the “top” surface of the rectangular sheet, functional strands were used to replace the original staple strands at the corresponding positions. Functional strands include fasteners for rolling tube origami nanostructures, capture strands for cargo loading, additional aptamer-containing strands for targeting delivery and fluorescent dye-labeled strands for imaging. Strands in red and blue are fasteners (48, 73, 97, 120, 144, and 169) to form the tubular configuration. Twelve thrombin-loading strands (yellow; 43, 44, 57; 64, 65, 78; 139, 140, 153; 160, 161 and 174) are extended at their 5′-end with ssDNA composed of 4 binding sites to capture thrombin-DNA molecules. Eight additional targeting strands (green; 1, 12, 205, 216) with 5′-end extended AS1411 sequences (G-quadruplex format) are placed at the four corners of the rectangle to increase targeting ability. For imaging, thirty-seven imaging strands (magenta) contain extended ssDNA sequences at their 5′-ends. These extensions are complementary to fluorescent dye-labeled ssDNA (the extended parts of the strands are not depicted in the figure).

FIG. 21. Functional strand configuration for thrombin loading on the “bottom” surface of the DNA-origami rectangle. Staple strands were reconfigured to form four bottom side thrombin-DNA binding sites (purple). Strands in red are fasteners. Strands in green are the additional targeting sequences.

FIG. 22. Schematic illustration of the synthesis of oligonucleotides conjugated to thrombin.

FIG. 23. Estimation of the concentration and DNA labeling ratio of the purified DNA-conjugated thrombin by measuring the absorbance at 260 and 280 nm. It is estimated that the polyT DNA-labeled thrombin has an average DNA-to-protein ratio of 2.5±0.8.

FIGS. 24A-24B. Characterization of free thrombin and thrombin-DNA conjugates. (FIG. 24a) Free thrombin (Th) and thrombin-DNA conjugate (Th-DNA) were resolved by SDS-PAGE under non-reducing conditions. Bands were detected by staining with Coomassie Blue. The larger conjugate (approximately 52 kD) showed a slower mobility relative to free thrombin. (FIG. 24b) The biological activity of thrombin or thrombin-DNA conjugate was determined by analyzing their ability to hydrolyze chromozym TH. Data represent the mean±s.d. of three independent experiments.

FIG. 25. Additional AFM images showing thrombin binding to the surface of DNA sheets. The white circles indicate the thrombin on the DNA origami sheets.

FIG. 26. Representative large scale AFM images of thrombin-DNA sheet assemblies. The majority of the fabricated DNA sheets showed four bright spots on the surface, representing the thrombin molecules. Blue arrows highlight the DNA nanosheets containing 4 thrombin molecules.

FIG. 27. Histograms of the numbers of thrombin assembled on the DNA origami sheet from the AFM images in FIG. 26. The efficiency of thrombin loading was calculated from AFM images by analyzing the number of thrombin on rectangular DNA origami sheets. Approximately 75% of the sheets contained four bound thrombin molecules.

FIG. 28. The DNA sheets co-migrate with Cy5-S15-Thrombin, indicating the binding of thrombin molecules to the DNA origami nanostructures.

FIGS. 29A-29F. Additional characterization of DNA origami nanostructures. (FIGS. 29a,b) Schematic diagram showing the rectangular (FIG. 29a) and tubular (FIG. 29b) origami structures, with dimensions of 90 nm×60 nm×2 nm for the rectangle and a diameter of 19 nm for the tube. For the formation of the tube structure, a DNA aptamer-based fastener (dark red) was attached along the two long sides of the rectangular nanostructure. Unless otherwise noted, the fastener duplex length was 16 bp. Additional free AS1411 aptamer (yellow) was conjugated to both ends of the tube to facilitate enhanced targeting. (FIG. 29c) Upper panels: AFM micrographs showing DNA origami sheet (left) and empty DNA nanorobot (right); lower panels: TEM images of DNA origami in the rectangle (left) and tube (right) configurations. Scale bars, 200 nm. (FIG. 29d) Dynamic light scattering (DLS) was used to analyze the distribution of hydrodynamic diameters of the rectangular and tubular DNA nanostructures in TAE/Mg buffer. (FIGS. 29e,f) The heights of the DNA origami sheet-Th (FIG. 29e) and nanorobot-Th (FIG. 29f) were measured by AFM. Scale bars of the zoomed AFM images, 50 nm.

FIGS. 30A-30C. Schematic of curvature validation and platelet aggregation assay using origami-thrombin complexes. (FIGS. 30a,b) Thrombin molecules are captured on the “top” (FIG. 30a) or “bottom” (FIG. 30b) surfaces of the rectangular DNA origami structure. Fasteners are then added to form the tube DNA nanostructure. A platelet aggregation assay was used to determine the percent of thrombin molecules inside the S11 tubes. Proteinase K was applied to remove the thrombin molecules outside the tubes. In brief, a proteinase K compact reaction column was prepared by covalently immobilizing proteinase K onto cyanogen bromide activated sepharose resins (Sigma-Aldrich, St. Louis, Mo., catalog No. C9142) that served as the chromatography medium. To digest the thrombin molecules outside the nanotubes, DNA nanorobot samples were run through the proteinase K-sepharose column. The nanorobot samples without exposed thrombin were then collected and degraded with DNaseI. The platelet aggregation assay was applied to the degraded products to determine the percent of thrombin molecules that were inside the tubes. (FIG. 30c) Platelet aggregation assay using origami-thrombin complexes. Aggregation (%) represents the percentage of platelets that were in the aggregated state. Free thrombin and rect-DNA-origami-Th (all loaded thrombin molecules are exposed and active) were used as positive controls. Blank rect-DNA-origami was used as a negative control. Samples without proteinase K and DNase I treatment showed the aggregating activity of thrombin on the outside surface of tubes while those with both treatment demonstrated the aggregating activity of thrombin on the inside surface of tubes. These data indicate that thrombin loaded inside the tube structure was approximately 84% for DNA nanorobot-Th, while thrombin loaded inside the tube structure was approximately 16% for the ctrl-Tube-DNA-origami-Th. Representative data of five separate experiments using platelets from different donors are shown as mean±s.d. of triplicates carried out in a single experiment.

FIG. 31. Predicted secondary structures and sequence of fasteners. To examine whether the length of the AS1411 duplex affected the dissociation of the fastener in response to nucleolin, we prepared fasteners containing either a 15 base pair duplex (F50+Comp15) or a 26 base pair duplex (F50+Comp26). Comp15 is partially complementary to the AS1411 sequence of F50, while Comp26 is fully complementary. Predicted secondary structures are shown for the 15-bp-duplex and 26-bp-duplex Y-shaped DNA strands. The region of the AS1411 aptamer responsible for binding to nucleolin is outlined in red. Only for the 15 bp duplex could the construct be stabilized in the dissociated state by the aptamer receptor, nucleolin.

FIGS. 32A-32E. Surface nucleolin expression on HUVECs and F50 binding specificity. (FIG. 32a) Nucleolin expression on the surface of HUVECs was assessed by flow cytometry using an antibody specific to human nucleolin. (FIG. 32b) Quantification of the flow cytometry data in FIG. 32a. Data are shown as mean±s.d. of three independent experiments. (FIGS. 32c-e) We next tested the ability of F50 to bind to the surface of HUVECs to confirm the function of the AS1411 sequence. HUVECs were treated with FITC-labeled F50 (15 μM) in the presence or absence of an antibody specific for human nucleolin. The cells were then subjected to flow cytometry. In contrast to the random DNA sequence (FIG. 32c), a specific binding of F50 to HUVECs (FIG. 32d) was observed. The specificity of F50 binding was confirmed by competitive blockage of cell-surface nucleolin using a specific antibody (Ab) to nucleolin (FIG. 32e). Unstained cells are shown in green. The data are representative of three independent experiments.

FIGS. 33A-33C. Binding of duplexes of different lengths to the surface of HUVECs. FITC-labeled F50 (FIG. 33a), 15 bp duplexed-F50 (FIG. 33b) or 26 bp duplexed-F50 (FIG. 33c) was incubated with HUVECs at 37° C. for different time periods. The cells were then analyzed by flow cytometry. The 15 bp duplex structure was able to effectively bind to the HUVEC surfaces similarly to free F50 so was chosen for subsequent experiments. Unstained cells are shown in green. The data are representative of three independent experiments.

FIG. 34. DNA fastener-triggered activation by recombinant nucleolin. FITC-labeled F50 containing AS1411 sequences exhibit enhanced fluorescence intensity after the Y-shaped fastener dissociates when incubated with recombinant nucleolin. Nucleolin treated with fasteners of partially (15 bp, red line) and fully complementary (26 bp, magenta line) AS1411 duplex are shown.

FIG. 35. DNA nanorobot activation triggered by recombinant nucleolin. In the “fastened” state, the fluorescence is quenched. When the nanotubes open, an increase in fluorescence intensity is expected. Fluorophore-quencher pair-labeled nanorobots were treated with recombinant nucleolin. A significant increase in fluorescence intensity was observed, indicating the opening of the nanorobots.

FIG. 36. Plasma membrane nucleolin in non-starved and serum-starved HUVECs. Immunoblot of nucleolin and Na+-K+-ATPase (plasma membrane marker) in plasma membrane fractions purified using a Membrane Protein Extraction Kit (ThermoFisher Scientific, catalog No. 89842). The anti-nucleolin antibody specifically binds bands at 110 and 75 kD in non-starved cells. These bands are almost undetectable in serum-starved cells. The lower molecular mass forms of nucleolinare fragments that result from the self-cleaving activity of nucleolin. The absence of actin indicates successful isolation of the plasma membranes. The results are representative of three separate experiments, all of which yielded similar results.

FIG. 37. Relationship between the concentration of exposed thrombin and plasma coagulation time. There is a linear relationship between the rate of plasma coagulation and the concentration of nanorobot-Th added to the cells. Data represent the mean±s.d. of five independent measurements.

FIGS. 38A-38B. Additional AS1411 aptamer strands enhance the cell binding ability of tubular DNA origami structures. (FIG. 38a) Confocal images showing the binding of Alexa 594-labeled empty DNA nanorobot with 2, 4 or 8 additional AS1411 aptamer strands to HUVECs after incubation for 1 h at 37° C. Nanotube DNA origami structures without additional aptamer strands were used as a control. Scale bars, 20 μm. (FIG. 38b) Flow cytometry analysis of the binding of FITC-labeled nanotube DNA origami structures with various numbers of additional AS1411 aptamer strands to HUVECs. The tube DNA origami structures exhibited enhanced cell binding as the number of additional AS1411 aptamer strands increased. The data are representative of three independent experiments.

FIGS. 39A-39D. Stability of DNA nanorobot-Th nanostructures. (FIG. 39a) Agarose gel image of DNA nanorobot-Th in the absence or presence of 0.4% bovine serum albumin (BSA) in PBS. DNA nanorobot-Th structures show a uniform and stable band distribution over time. (FIG. 39b) Distribution of hydrodynamic diameter for DNA nanorobot-Th nanoparticles in 0.4% BSA in PBS, as determined by DLS. Data represent the mean±s.d. of three independent measurements. (FIG. 39c) Agarose gel image of DNA nanorobot in the absence or presence of 10% fetal bovine serum (FBS) in PBS. The nanostructures were stable over 24 h. (FIG. 39d) Coagulation induction by FBS-treated DNA nanorobot-Th. DNA nanorobot-Th nanostructures were mixed with FBS and incubated at 37° C. for 0, 4 and 24 h. The incubation products (100 μl, equivalent to ˜0.5 U thrombin) were added to the wells of a 24-well plate (Corning, Woburn, Mass., USA). Mouse plasma prepared from blood collected with 3.8% sodium citrate solution (anticoagulant; citrate/blood: 1/9, v/v) was added to the mixture, and the duration until the first fibrin strand formation was noted. The plasma only group was used as a negative control, and free thrombin as a positive control. Error bars represent the mean±s.d. of three independent experiments.

FIGS. 40A-40C. In vivo tumor targeting and biodistribution of targeted and nontargeted DNA nanostructures. Nude mice bearing MDA-MB-231 human breast tumors were administered Cy5.5-labeled nanorobots, nontargeted nanotubes, targeted nanotubes (targeted but without the ability to open) and free Cy5.5 via a single tail vein injection. (FIG. 40a) Photographs showing the fluorescence distribution in mice at the indicated time points after injection. At 8 h post-injection, a high-intensity fluorescent signal was detected in the tumor region of mice treated with targeted nanorobots or nanotubes but not nontargeted nanotubes or free dye. Tumors are indicated by red circles. (FIG. 40b) The tumor accumulation of targeted nanorobots was ˜7 times more than that of nontargeted nanotubes at 8 h after injection; n=3. (FIG. 40c) Ex vivo representative fluorescence images of major organs following administration of different DNA nanostructures or free dyes to tumor-bearing mice at the indicated time points; n=3.H, heart; Li, Liver; S, spleen; Lu, lung; K, kidney; T, tumor.

FIGS. 41A-41C. Representative immunohistochemical and H&E staining of tumors and normal tissues. (FIG. 41a) MDA-MB-231 tumor-bearing mice were injected intravenously with saline, free thrombin, targeted empty nanorobot, nontargeted nanotube-Th or targeted nanotube-Th. The tumors harvested at 0 (before treatment), 24, 48 or 72 h administration were immunostained for CD41 (activated platelets) to detect thrombosis (brown). Scale bars, 50 μm. (FIG. 41b) Compared to the saline group, the heart, liver, lung and kidney from the nanorobot-Th treated mice after 72 h of injection showed no visible thrombosis. Biological replicates were used in three animals (n=3) in each study group. Scale bars, 500 μm. (FIG. 41c) The tumors harvested at 0 (before treatment), 24, 48 or 72 h administration of various control vehicles were subjected to H&E staining for necrotic tissues (N). Scale bars, 200 μm.

FIGS. 42A-42D. Treatment with nanorobot-Th inhibits melanoma growth and reduces tumor cell metastasis. (FIGS. 42a,b) B16-F10 cells were injected subcutaneously into the flanks of C57BL/6J mice. When tumors reached a size of ˜150 mm³, mice were treated on day 0 with saline, free thrombin, empty nanorobot or thrombin-loaded nanorobot (nanorobot-Th). Tumor volumes up to day 14 (FIG. 42a) and cumulative survival (FIG. 42b) of mice (n=10). Tumor volumes were compared using a Kruskal-Wallis test followed by a Mann-Whitney test. Cumulative survival curves were compared using Kaplan Meier analysis followed by the Log Rank test. *p<0.05, **p<0.01 and ***p<0.001, compared to the other groups. (FIGS. 42c,d) After eight treatments with saline or nanorobot-Th, the mice were sacrificed, and liver weight was determined (FIG. 42c). H&E staining of liver sections demonstrates the presence of metastases (M) in saline treated mice but not in the thrombin-loaded nanorobot group (FIG. 42d, top). Liver sections were further stained for the melanoma markers HMB-45 (FIG. 42d, middle, brown) and Melan-A (FIG. 42d, bottom, brown), and nuclei were indicated in blue. HMB-45 or Melan-A-positive melanoma cells were only seen in the livers of saline group. Scale bars, 50 μm.

FIGS. 43A-43E. Treatment with nanorobot-Th suppresses the growth of poorly permeable SK-OV3 xenografts. (FIG. 43a) Mice bearing ˜250 mm3SK-OV3 and MDA-MB-231 tumors were injected 100 μL 5% Evans blue intravenously. After 3 h, the tumors were removed and Evans blue was extracted by treating the tumor tissues with formamide for three days. The Evans blue content of the supernatants was measured using a UV-Vis spectrophotometer at 620 nm. Compared with MDA-MB-231 tumors, the lighter blue color (left; representative photographs) and lower Evans blue content (right) in SK-OV3 tumors indicate their lower permeability. (FIGS. 43b-e) SK-OV3 tumor-bearing mice were treated on day 0 with saline, free thrombin, a scrambled aptamer control (scramble), targeted empty nanorobot, nontargeted nanotube-Th, targeted nanotube-Th or nanorobot-Th. Tumor volumes up to day 21 (FIG. 43b) were compared using a Kruskal-Wallis test followed by a Mann-Whitney test (n=8). Tumors excised at day 21 were photographed (FIG. 43c) and weighed (FIG. 43d). (FIG. 43e) Cumulative survival of SK-OV3 tumor-bearing mice (n=10) compared using Kaplan Meier analysis followed by the Log Rank test. All treatments started when the tumor volume reached ˜100 mm3. **p<0.01, ***p<0.001.

FIGS. 44A-44D. Nanorobot-Thsuppresses the progression of doxycycline-induced lung tumors in TetO-KRASG12D transgenic mouse model. (FIG. 44a) Mice bearing doxycycline-induced lung tumors (2-week induction) were treated with saline, free thrombin, nontargeted nanotube-Th and nanorobot-Th. Representative Mill images taken before, one week and two weeks after the treatment started were shown. Tumor growth was visibly inhibited in the mice treated with nanorobot-Th, compared to the mice treated with the controls. Scale bars, 1 cm. (FIG. 44b) Statistical analysis of tumor burden shown in (FIG. 44a). Data are shown as mean±s.d. of three mice. *p<0.05, **p<0.01. (FIG. 44c) Lung organs were harvested after 2-week treatment and weighed. Error bars represent the mean±s.d. of three mice. **p<0.01. (FIG. 44d) Representative lung sections stained with H&E from mice after 2-week treatment with various vehicles. Scale bars, 200 μm.

FIGS. 45A-45F. Thrombotic risk assessment in mice. (FIG. 45a) MDA-MB-231 tumor-bearing mice were given 0, 1.15, 2.30, 3.45, 4.60 and 15.0 U thrombin/mouse (i.v.). Cerebral microthrombi were detected using the cranial window technique that has been previously shown not to cause artificial damage to normal blood flow. Injection with 3.45 U or more of thrombin developed transient and reversible microthrombi as indicated by red arrowheads. White arrowheads indicate venules. Scale bars, 100 μm. (FIG. 45b-e) The percentage of P-selectin-positive platelets (FIG. 45b), total fibrin (FIG. 45c) and thrombin (FIG. 45d) levels and platelet blood count (FIG. 45e) in the plasma of MDA-MB-231 tumor-bearing mice treated by single (0.5, 2, 4 and 24 h) or six injections (18d) of DNA nanorobot-Th. Data represent the mean±s.d. of three independent experiments. 0 h, before injection. NS, not significant. (FIG. 45f) Microthrombi detection in cerebral vessels of mice after DNA nanorobot-Th treatment. Mice bearing MDA-MB-231 tumors were given single intravenous injections of DNA nanorobot-Th. Imaging analysis to reveal microthrombi in cerebral venous vessels was performed at the indicated time points. Free thrombin (5.0 U/mouse) was used as a positive control. Red arrows indicate microthrombi, which were only detected when free thrombin was administered. Biological replicates were used and the study was repeated three times in the laboratory. Scale bars, 100 μm. NS, not significant.

On the microthrombosis-related risk of the DNA nanorobot, it should be also noted that, while tumor blood vessels are mainly capillary-sized vessels which are easily occluded by microthrombi, in normal vessels even if a few microthrombi break off they will be quickly cleared in the bloodstream as reported previously [5, 6]. The low equivalent concentration of thrombin used in the nanorobot-Th therapy further reduced the potential risk of undesirable microthrombi causing damage to normal tissues.

FIG. 46. Serum cytokine concentrations in mice treated with DNA nanorobot-Th. Serum cytokine concentrations of non-tumor-bearing C57BL/6J mice treated with single (2 h and 48 h) and six injections (18 d) of DNA nanorobot-Th. Data represent the mean±s.d. from five animals in each group. 0 h, before injection. NS, not significant. Biological replicates were used and the study was repeated at least two times in the laboratory.

FIG. 47. The effect of DNA nanorobot-Th on cell viability. bEnd3 cells were treated with DNA nanorobot-Th at either 3.3 or 6.6 nM. Cell viability was determined by a CCK-8 cytotoxicity assay after incubation for 24, 48 or 72 h. Error bars represent the mean±s.d. of five independent experiments.

FIGS. 48A-48F. Risk assessment of thrombosis by DNA nanorobot-Thin Bama miniature pigs. (FIG. 48a) Gross morphology of the healthy pigs. (FIGS. 48b,c) H&E staining of major organs of pigs treated with 150 U thrombin/pig (FIG. 48b) and 350 U thrombin/pig (FIG. 48c). After three injections, representative tissue staining images show that no thrombosis was observed in either group. Biological replicates were used and the study was repeated three times in the laboratory. Scale bars, 100 μm. (FIGS. 48d,e) Minipigs were treated with nanorobot-Th every other day for a total of three injections. Blood was collected before injection (0 h) and after single (2 h and 24 h), two (3 d) and three (5 d) injections. Prothrombin time (PT), activated partial thromboplastin time (APTT), and thrombin time (TT) values (FIG. 48d) and the levels of fibrinogen protein and D-dimer (FIG. 48e) in the plasma were then measured. Plasma samples obtained before the first injection were used as a control. Data are mean±s.d.; n=3. NS, not significant. (FIG. 48f) H&E staining of major normal tissues of pigs treated with three injections of DNA nanorobot-Th showed no thrombosis. Scale bars, 100 μm.

The following formula was used to perform dose conversion between mice and pigs [7,8]: Dp=Dm×(Kmm/Kmp). Where Dp is the dose injected into pigs, Dm is the dose used in mice, Kmm is the Dose in mg/kg to Dose in mg/m² conversion factor of mice and Kmp is the Dose in mg/kg to Dose in mg/m² conversion factor of the pigs.

### DETAILED DESCRIPTION

Robotic molecular systems have great potential as intelligent vehicles to enable the delivery of various potent molecules, which otherwise never could be used as therapeutics due to numerous limitations. Yet, achieving in vivo, precise molecular-level, and on-demand targeting and delivery has proven extremely challenging. An autonomous DNA robotic system was developed for targeted cancer therapy, programmed to transport molecular payloads and cause on-site tumor infarction.

In certain embodiments, a nanorobot, functionalized with tumor endothelium-specific DNA aptamers on its external surface, and the blood coagulation protease thrombin within its inner cavity, initiated tumor vessel occlusion and induces tumor necrosis. Due to the specific expression of nucleolin on the surface of tumor endothelial cells, nucleolin-targeting aptamers serve as both targeting and trigger molecules for the mechanical opening of the DNA nanorobot to expose thrombin molecules and activate coagulation at the tumor site. Using tumor-bearing mouse models of breast cancer and melanoma, it was demonstrated that intravenously injected DNA nanorobots delivered thrombin specifically to the tumor-associated vessels and induces intravascular thrombosis, resulting in tumor necrosis and inhibition of tumor growth. The nanorobot proved to be safe and immunologically inert for use in normal mice and Bama miniature pigs, eliciting no detectable changes in blood coagulation parameters and histological morphology in either model. Given its robust self-assembly behavior, exceptional designability, potent antitumor activity and minimal in vivo adversity, this DNA nanorobot represents a promising strategy for precise drug/therapeutic agent design for cancer therapeutics.

### DNA Nanostructures, DNA Nanostructure Nanorobots and Compositions Thereof

In certain embodiments, the DNA nanostructure nanorobot is comprised of one DNA scaffold strand, a plurality of staple strands, and functional strands of DNA, such as fasteners, and, optionally, a targeting, imaging or capture strands of DNA that are operably linked to the DNA scaffold. The different elements of the DNA nanobot are capable of self-assembling into a nanostructure. For example, in certain embodiments, the single stranded DNA molecule is M13 phage single stranded DNA and staple strand, as described herein. As described in the Example, this nanostructure may be used as a carrier for a molecular payload, including inducing anti-tumor vascularization effects.

In certain embodiments, the present invention provides a DNA nanostructure nanorobot comprising:

a single stranded DNA scaffold strand of about 5000 to 10,000 bases in length;

a plurality of staple strands of DNA of about 20 to 40 bases in length, wherein each staple strand has a unique sequence and is hybridized to a specific position on the DNA scaffold strand, wherein the plurality of staple strands hybridized to the DNA scaffold form a sheet having a top surface and a bottom surface, and having four corners;

one or more fastener strands of DNA, wherein the one or more fastener strands of DNA is capable of fastening the sheet into a tube-shaped origami structure.

In certain embodiments, the DNA nanostructure nanorobot further comprises DNA targeting strands, wherein each targeting strand is operably linked to a targeting moiety. In certain embodiments, the targeting moiety is an aptamer. In certain embodiments, the aptamer is specific for nucleolin.

In certain embodiments, the DNA nanostructure nanorobot further comprises DNA imaging strands, wherein each imaging strand is operably linked to an imaging agent. In certain embodiments, the imaging agent is fluorescent dye.

As used herein, the term “DNA nanostructure” refers to a nanoscale structure made of DNA, wherein the DNA acts both as a structural and function element. DNA nanostructures can also serve as a scaffold for the formation of other structures. DNA nanostructures may be prepared by methods known in the art using one or more nucleic acid oligonucleotides. For example, in certain embodiments, the DNA nanostructure is an DNA rectangle origami nanostructure, self-assembled from single-stranded DNA molecules using “staple strands.”

The length of the single stranded DNA scaffold strand is variable and depends on, for example, the type of nanostructure. In certain embodiments, the DNA scaffold strand is comprised of multiple oligonucleotide strands. In certain embodiments, the DNA scaffold strand is comprised of a single oligonucleotide strand. In certain embodiments, the DNA scaffold strand is about nucleotides in length to about 10000 nucleotides in length.

For use in the present invention, the nucleic acids can be synthesized de novo using any of a number of procedures well known in the art. For example, the cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622, 1988). These chemistries can be performed by a variety of automated oligonucleotide synthesizers available in the market, including the use of an in vitro transcription method.

In certain embodiments, the DNA nanostructure has increased nuclease resistance (e.g., as compared to a control, such as an unfolded ssDNA molecule comprising the same nucleic acid sequence as the DNA nanostructure). In certain embodiments, nuclease resistance of the DNA nanostructure is 10%, 20%, 30%, 40%, 50%, 60%, 70%, 80%, 90%, 95% or more than a control.

In certain embodiments, the DNA nanostructure is assembled using a single stranded DNA molecule as an initial scaffold. In certain embodiments, the DNA nanostructure comprises both single stranded and double stranded regions.

In certain embodiments, the present invention provides a DNA nanostructure nanorobot comprising:

a single stranded DNA scaffold strand of about 5000 to 10,000 bases in length;

a plurality of staple strands of DNA of about 32 bases in length, wherein each staple strand has a unique sequence and is hybridized to a specific position on the DNA scaffold strand, wherein the plurality of staple strands hybridized to the DNA scaffold form a rectangular sheet having a top surface and a bottom surface, and having four corners;

one or more fastener strands of DNA, wherein the one or more fastener strands of DNA is capable of fastening the rectangular sheet into a tube-shaped origami structure; and

one or more DNA capture strands, wherein each capture strand is operably linked to a therapeutic agent.

In certain embodiments, the DNA nanostructure nanorobot further comprises DNA targeting strands, wherein each targeting strand is operably linked to a targeting moiety. In certain embodiments, the targeting moiety is an aptamer.

In certain embodiments, the DNA nanostructure nanorobot further comprises DNA imaging strands, wherein each imaging strand is operably linked to an imaging agent. In certain embodiments, the imaging agent is fluorescent dye.

As used herein, “staple strands” are short single-stranded oligonucleotides of about 20 to about 40 nucleotides in length, such as 20, 21, 22, 23, 24, 25, 26, 27, 28, 29, 30, 31, 32, 33, 34, 35, 36, 37, 38, 39, or 40 nucleotides in length, wherein one end of the staple strand hybridizes with a region of the scaffold strand, and the second end of the staple strand hybridizes with another region of the scaffold strand, thereby “stapling” the two regions of the scaffold strand. Exemplary staple strands are provided below as Staple Strands 13-204. In certain embodiments, the dimension of the rectangular sheet is about 90 nm×about 60 nm×2 nm.

In certain embodiments, the tube-shaped origami structure has a diameter of about about 19 nm.

In certain embodiments, the fastener strand is a Y-shaped structure. In certain embodiments, the Y-shaped structure comprises an F50 AS1411 aptamer sequence that specifically binds to nucleolin, and a Comp15 DNA strand partially complementary to the AS1411 sequence, wherein the F50 and the Comp15 sequences form a 14- to 16-base pair duplex.

In certain embodiments, the Y-shaped structure comprises 5′-FITC-labeled F50 and 3′-BHQ1-labeled Comp15; FITC-F50-48 and Comp15-48-Q; FITC-F50-73 and Comp15-73-Q; FITC-F50-97 and Comp15-97-Q; FITC-F50-120 and Comp15-120-Q; FITC-F50-144 and, Comp15-144-Q; or FITC-F50-169 and Comp15-169-Q.

In certain embodiments, the capture strand is extended with ssDNA comprising four binding sites to “capture” thrombin-DNA molecules.

In certain embodiments, DNA nanostructure robot further comprises one or more functional strand of DNA operably linked to an aptamer for targeting delivery of the nanorobot forming a targeting strand. {NINA]

In certain embodiments, the aptamer is specific for nucleolin.

In certain embodiments, one or more targeting strands are positioned at one or more corners of the rectangular sheet.

In certain embodiments, one or more capture strands is operably linked to a fluorescent dye to form an imaging strand.

In certain embodiments, the therapeutic agent is operably linked to the top surface of the rectangular sheet.

In certain embodiments, the therapeutic agent is operably linked to the bottom surface of the rectangular sheet.

In certain embodiments, the therapeutic agent is operably linked to an imaging agent. Imaging agents are well-known in the art and any can be operably linked to a therapeutic agent. In certain embodiments, the imaging agent is a fluorescent dye.

In certain embodiments, the therapeutic agent is a protein.

In certain embodiments, the therapeutic agent is thrombin.

In certain embodiments, the therapeutic agent is siRNA, a chemotherapeutic agent or a peptide therapeutic agent.

In certain embodiments, the thrombin is operably linked to the functional strand of DNA by means of a sulfosuccinimidyl-4-(N-maleimidomethyl) cyclohexane-1-carboxylate (sulfo-SMCC) as a bifunctional crosslinker.

In certain embodiments, the nanorobot comprises four thrombin molecules.

In certain embodiments, the target molecule is nucleolin.

In certain embodiments, the thrombin is operably linked to an imaging agent. In certain embodiments, the imaging agent is a fluorescent dye.

Certain embodiments of the invention provide a pharmaceutical composition comprising the DNA nanostructure nanorobot described herein.

In certain embodiments, the composition further comprises at least one therapeutic agent.

In certain embodiments, the at least one therapeutic agent is a chemotherapeutic drug (e.g., doxorubicin).

Certain embodiments of the invention provide a method of treating a disease or disorder in a subject, comprising administering to the subject a therapeutically effective amount of the DNA nanostructure nanorobot or pharmaceutical composition as described herein.

In certain embodiments, the disease or disorder is cancer.

In certain embodiments, the cancer is breast cancer, ovarian cancer, melanoma or lung cancer.

Certain embodiments of the invention provide a use of the DNA nanostructure nanorobot or a composition as described herein for the manufacture of a medicament for inducing an tumor necrosis response in a subject (e.g., a mammal, such as a human).

Certain embodiments of the invention provide a DNA nanostructure nanorobot or a composition as described herein for the prophylactic or therapeutic treatment a disease or disorder.

Certain embodiments of the invention provide a kit comprising the DNA nanostructure nanorobot or a composition as described herein and instructions for administering the DNA nanostructure nanorobot/composition to a subject to induce an immune response or to treat a disease or disorder. In certain embodiments, the kit further comprises at least one therapeutic agent.

The invention also provides processes disclosed herein that are useful for preparing a DNA nanostructure nanorobot described herein.

In certain embodiments, one or more agents (e.g., 1, 2, 3, 4, 5, 6, 7, 8, 9, 10 etc.) may be operably linked to the DNA nanostructure, such as diagnostic agents or therapeutic agents. In certain embodiments, at least one diagnostic agent is operably linked to the DNA nanostructure. In certain embodiments, at least one therapeutic agent is operably linked to the DNA nanostructure. In certain embodiments, at least one diagnostic agent and at least one therapeutic agent are operably linked to the DNA nanostructure. Diagnostic agents are known in the art and include, e.g., fluorophores and radioisotopes, colorimetric indicator.

As used herein, the term “therapeutic agent” includes agents that provide a therapeutically desirable effect when administered to an animal (e.g., a mammal, such as a human). The agent may be of natural or synthetic origin. For example, it may be a nucleic acid, a polypeptide, a protein, a peptide, a radioisotope, saccharide or polysaccharide or an organic compound, such as a small molecule. The term “small molecule” includes organic molecules having a molecular weight of less than about, e.g., 1000 daltons. In one embodiment a small molecule can have a molecular weight of less than about 800 daltons. In another embodiment a small molecule can have a molecular weight of less than about 500 daltons.

In certain embodiments, the therapeutic agent is an immuno-stimulatory agent, a radioisotope, a chemotherapeutic drug (e.g., doxorubicin) or an immuno-therapy agent, such as antibody or an antibody fragment. In certain embodiments, the therapeutic agent is a vaccine, such as a cancer vaccine. In certain embodiments, the therapeutic agent is a tumor targeting agent, such as a monoclonal tumor-specific antibody or an aptamer. In certain embodiments, the therapeutic agent is an antibody (e.g., a monoclonal antibody, e.g., an anti-PD1 antibody). In certain embodiments, the therapeutic agent is an antigen (e.g., a tumor associated antigen or a tumor specific antigen). In certain embodiments, the therapeutic agent is a tumor antigen peptide(s). In certain embodiments, the therapeutic agent is thrombin.

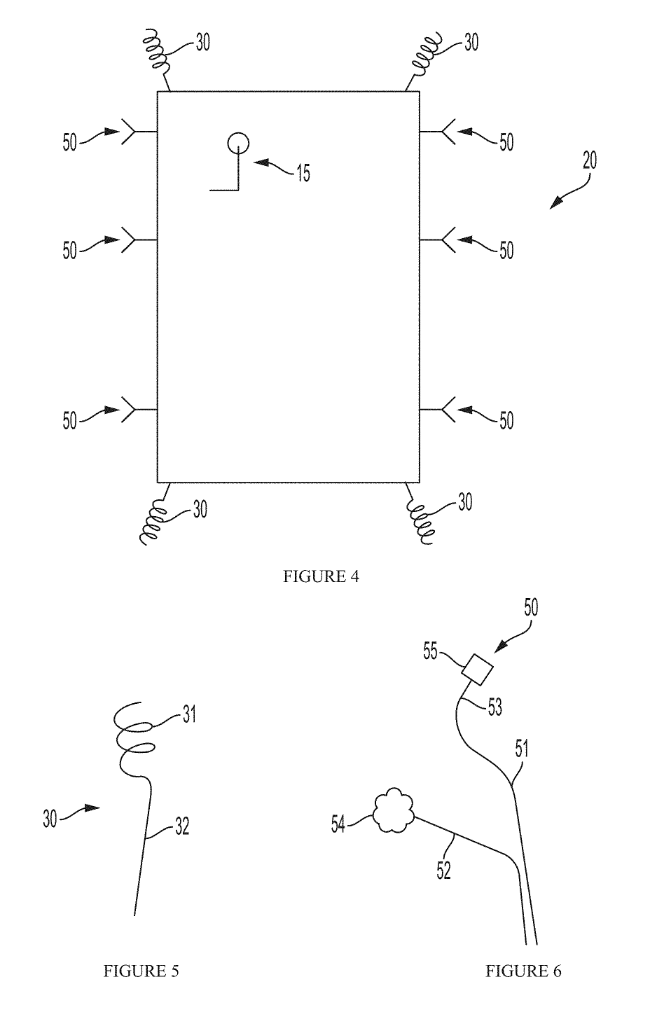

As shown in FIG. 4, in certain embodiments the scaffold is an unfolded rectangular DNA origami structure 20 having fasteners 50 extending from the edges that can be joined, aptamer-containing targeting strands and a therapeutic agent-DNA conjugate capture strand. In certain embodiments, the DNA origami structure 20 also has aptamer-containing targeting strands 30 attached thereto.

As shown in FIG. 5 in certain embodiments the aptamer-containing targeting strand 30 containing an aptamer portion 31 and an attaching DNA strand portion 32.

As shown in FIG. 6 in certain embodiments the fastener 50 has two arms 51 and 52, and having a quencher moiety 54 attached to one arm 52 of the Y-structure and a fluorophore moiety 55 attached to the second arm 51 of the Y-structure by means of a linker 53.

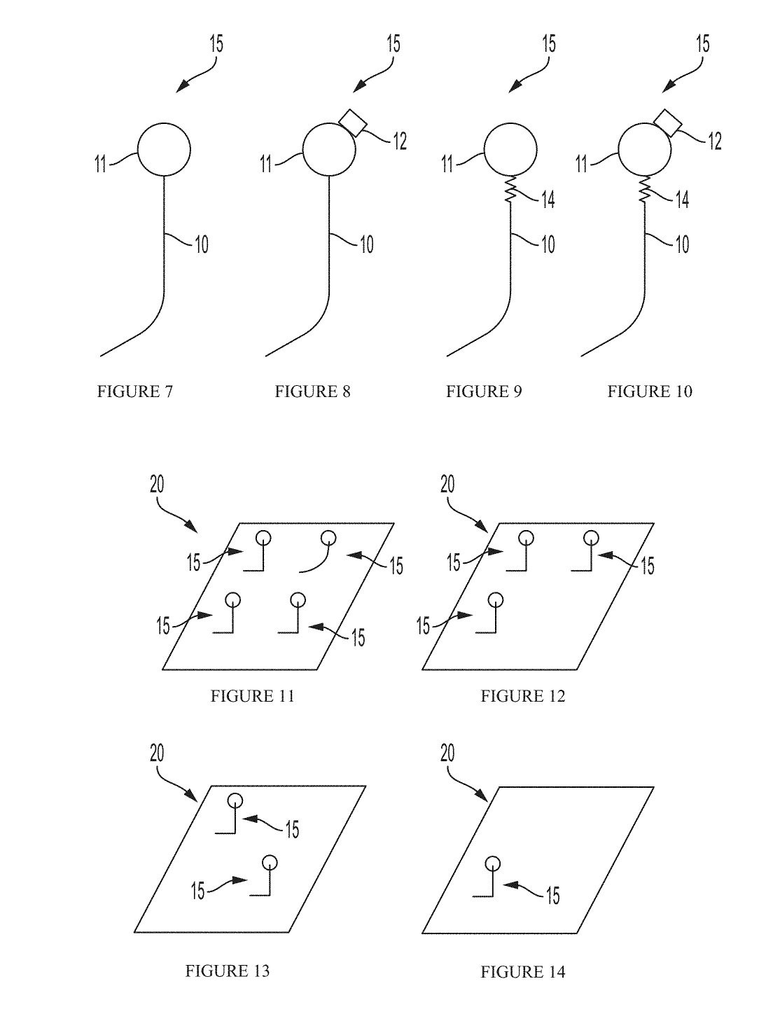

As shown in FIG. 7 in certain embodiments the therapeutic agent-DNA conjugate capture strand 15 has a ssDNA attachment strand 10 and a therapeutic agent payload 11. As shown in FIG. 8 in certain embodiments the therapeutic agent payload 11 is operably linked to an imaging agent 12. As shown in FIG. 9 in certain embodiments the ssDNA attachment strand 10 is linked to a therapeutic agent payload 11 by means of a linker 14. As shown in FIG. 10 in certain embodiments therapeutic agent-DNA conjugate capture strand having a ssDNA attachment strand that is linked to a therapeutic agent payload by means of a linker, where the therapeutic agent payload is operably linked to an imaging agent 12.

As shown in FIGS. 11-14, in certain embodiments the unfolded rectangular DNA origami structure has one to four therapeutic agent-DNA conjugates operably linked to the origami structure. The therapeutic agent-DNA conjugates can be attached to either the “top” or the “bottom” (or both) of the origami structure, such that when the origami structure is rolled into a tube, the therapeutic agent-DNA conjugates can be designed to be either on the inside or outside of the tube.

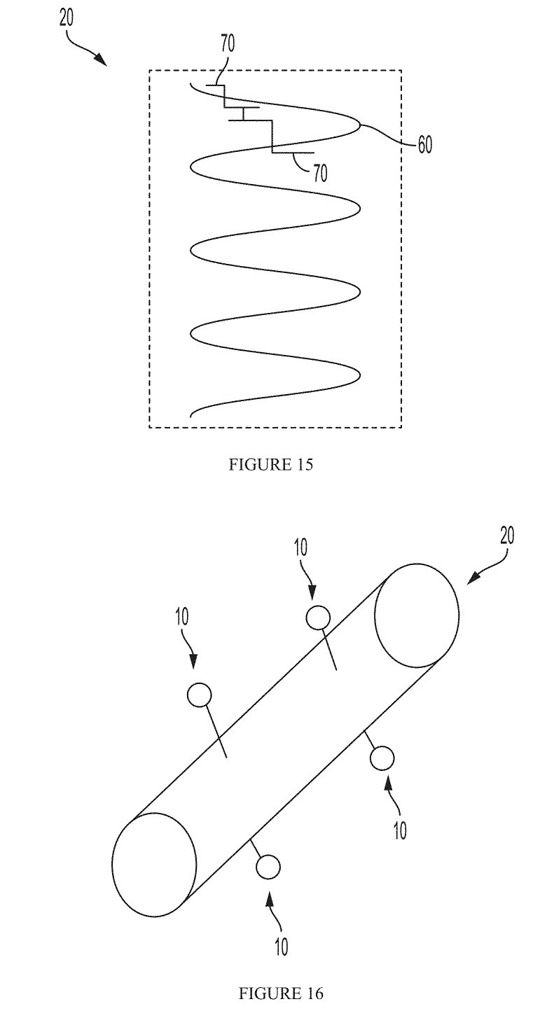

FIG. 15 depicts an exploded view of the DNA origami structure 20, detailing the hybridization of a single stranded DNA scaffold strand 60 and staple strands 70, and the interaction of the two staple strands.

As shown in FIG. 16 in certain embodiments, the tube-shaped DNA origami structure have therapeutic agent-DNA conjugates 10 positioned on the outside of the tube-shaped DNA origami structure.

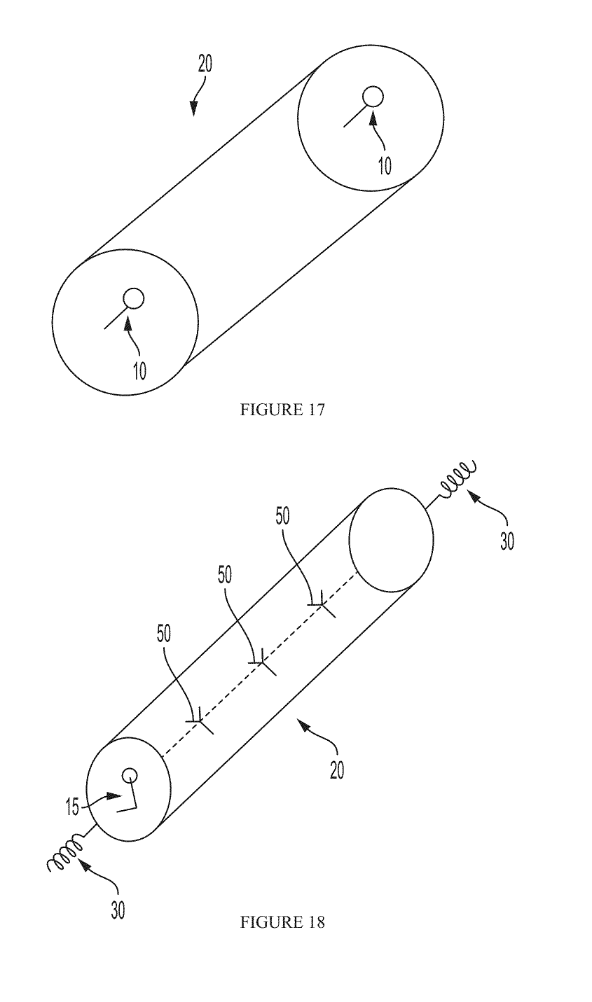

As shown in FIG. 17 in certain embodiments, the tube-shaped DNA origami structure having therapeutic agent-DNA conjugates 10 positioned on the inside of the tube-shaped DNA origami structure.

As shown in FIG. 18 depicts a tube-shaped DNA origami structure 20 having therapeutic agent-DNA conjugates 15 positioned on the inside of the tube-shaped DNA origami structure, having aptamer-containing targeting strands 30 positioned at the ends of the tube, and illustrating the fasteners 50 joining the edges of the DNA origami structure so as to form a tube shape.

Linkages

The linkage between the agent(s) and the DNA nanostructure is not critical, and may be any group that can connect the DNA nanostructure and the agent using known chemistry, provided that is does not interfere with the function of the agent or the DNA nanostructure. Chemistries that can be used to link the agent to an oligonucleotide are known in the art, such as disulfide linkages, amino linkages, covalent linkages, etc. In certain embodiments, aliphatic or ethylene glycol linkers that are well known to those with skill in the art can be used. In certain embodiments phosphodiester, phosphorothioate and/or other modified linkages are used. In certain embodiments, the linker is a binding pair. In certain embodiments, the “binding pair” refers to two molecules which interact with each other through any of a variety of molecular forces including, for example, ionic, covalent, hydrophobic, van der Waals, and hydrogen bonding, so that the pair have the property of binding specifically to each other. Specific binding means that the binding pair members exhibit binding to each other under conditions where they do not bind to another molecule. Examples of binding pairs are biotin-avidin, hormone-receptor, receptor-ligand, enzyme-substrate probe, IgG-protein A, antigen-antibody, aptamer-target and the like. In certain embodiments, a first member of the binding pair comprises avidin or streptavidin and a second member of the binding pair comprises biotin.

Therapeutic Agents to be Administered

In certain embodiments, the therapeutic agent is thrombin.

### Compositions and Kits

Certain embodiments of the invention also provide a composition comprising a DNA nanostructure nanorobot described herein and a carrier. In certain embodiments, the composition comprises a plurality of DNA nanostructure nanorobots.

In certain embodiments, the composition further comprises at least one therapeutic agent known in the art. In certain embodiments, the composition is pharmaceutical composition and the carrier is a pharmaceutically acceptable carrier.

The present invention further provides kits for practicing the present methods. Accordingly, certain embodiments of the invention provide a kit comprising an DNA nanostructure described herein and instructions for administering the DNA nanostructure nanorobot to induce an immune response (e.g., anti-tumor immunity) or to treat a disease or condition. In certain embodiments, the kit further comprises a therapeutic agent described herein and instructions for administering the therapeutic agent in combination (e.g., simultaneously or sequentially) with the DNA nanostructure.

### Certain Methods

As described in the Example, a DNA nanostructure nanorobot described herein may be used to induce vascular occlusion, slow the increase of or reduce the tumor burden, slow the increase of or reduce tumor size, reduce or block blood circulation in a tumor, slow the increase or reduce tumor cell metastasis, reduce or inhibit proliferation of a tumor, or induce a tumor necrosis.

Accordingly, certain embodiments of the invention provide a method of inducing an immune response in a subject, comprising administering to the subject an effective amount of a DNA nanostructure nanorobot or composition as described herein.

In certain embodiments, the administration increases an immune response by at least about, e.g., 20%, 30%, 40%, 50%, 60%, 70%, 80%, 90%, or more (e.g., as compared to a control). Methods of measuring an immune response are known in the art, for example using an assay described in the Example. The phrase “inducing an immune response” refers to the activation of an immune cell. Methods of measuring an immune response are known in the art, for example using an assay described in the Example.

Certain embodiments of the invention also provide a method of treating a disease or disorder in a subject, comprising administering to the subject a therapeutically effective amount of a DNA nanostructure nanorobot or a composition as described herein.

As used herein, the term “disease or disorder” refers to any disease or disorder that would benefit from induction of an immune response, vascular occlusion, a slowing in the increase of or reduction in tumor burden, a slowing in the increase of or reduction tumor size, reduction or blocking of blood circulation in a tumor, a slowing in the increase or reduction of tumor cell metastasis, reduction or inhibition tumor proliferation, or induction of a tumor necrosis response and include cancer.

In certain embodiments, a method of the invention further comprises administering at least one therapeutic agent to the subject.

The at least one therapeutic that can be administered is any therapeutic agent that can be used in the treatment of the disease or disorder of interest and include the therapeutic agents described herein.

The at least one therapeutic agent may be administered in combination with the DNA nanostructure. As used herein, the phrase “in combination” refers to the simultaneous or sequential administration of the DNA nanostructure and the at least one therapeutic agent. For simultaneous administration, the DNA nanostructure and the at least one therapeutic agent may be present in a single composition or may be separate (e.g., may be administered by the same or different routes).

Certain embodiments of the invention provide a DNA nanostructure nanorobot or a composition as described herein for use in medical therapy.

Certain embodiments of the invention provide the use of a DNA nanostructure nanorobot or a composition as described herein for the manufacture of a medicament for inducing an immune response in a subject.

Certain embodiments of the invention provide the use of a DNA nanostructure nanorobot or a composition as described herein for the manufacture of a medicament for inducing an immune response in a subject, in combination with at least one therapeutic agent.

Certain embodiments of the invention provide a DNA nanostructure nanorobot or a composition as described herein for inducing an immune response.

Certain embodiments of the invention provide a DNA nanostructure nanorobot or a composition as described herein for inducing an immune response, in combination with at least one therapeutic agent.

Certain embodiments of the invention provide the use of a DNA nanostructure nanorobot or a composition as described herein for the manufacture of a medicament for treating a disease or disorder in a subject.

Certain embodiments of the invention provide the use of a DNA nanostructure nanorobot or a composition as described herein for the manufacture of a medicament for treating a disease or disorder in a subject, in combination with at least one therapeutic agent.

Certain embodiments of the invention provide a DNA nanostructure nanorobot or a composition as described herein for the prophylactic or therapeutic treatment a disease or disorder.

Certain embodiments of the invention provide a DNA nanostructure nanorobot or a composition as described herein for the prophylactic or therapeutic treatment of a disease or disorder, in combination with at least one therapeutic agent.

In certain embodiments, the cancer is breast cancer, melanoma, ovarian cancer, lung cancer, carcinoma, lymphoma, blastoma, sarcoma, or leukemia. In certain embodiments, the cancer is a solid tumor cancer.

In certain embodiments, the cancer is squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer (e.g., endocrine resistant breast cancer), colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia, or head and neck cancer. In certain embodiments, the cancer is breast cancer.

### Administration

As described herein, methods of the invention comprise administering a DNA nanostructure described herein, and optionally, a therapeutic agent to a subject. Such compounds (i.e., a DNA nanostructure and/or therapeutic agent) may be formulated as a pharmaceutical composition and administered to a mammalian host, such as a human patient in a variety of forms adapted to the chosen route of administration, i.e., orally or parenterally, by intravenous, intramuscular, intraperitoneal or topical or subcutaneous routes.

Thus, the compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained. The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver a compound to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of compounds can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compound may be conveniently formulated in unit dosage form. In one embodiment, the invention provides a composition comprising a compound formulated in such a unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### Certain Definitions

As used herein, the term “about” means ±10%.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

“Operably-linked” refers to the association two chemical moieties so that the function of one is affected by the other, e.g., an arrangement of elements wherein the components so described are configured so as to perform their usual function.

The term “nucleic acid” refers to deoxyribonucleotides or ribonucleotides and polymers thereof in either single- or double-stranded form, made of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues.

The terms “nucleotide sequence” and “nucleic acid sequence” refer to a sequence of bases (purines and/or pyrimidines) in a polymer of DNA or RNA, which can be single-stranded or double-stranded, optionally containing synthetic, non-natural or altered nucleotide bases capable of incorporation into DNA or RNA polymers, and/or backbone modifications (e.g., a modified oligomer, such as a morpholino oligomer, phosphorodiamate morpholino oligomer or vivo-mopholino). The terms “oligo”, “oligonucleotide” and “oligomer” may be used interchangeably and refer to such sequences of purines and/or pyrimidines. The terms “modified oligos”, “modified oligonucleotides” or “modified oligomers” may be similarly used interchangeably, and refer to such sequences that contain synthetic, non-natural or altered bases and/or backbone modifications (e.g., chemical modifications to the internucleotide phosphate linkages and/or to the backbone sugar).

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methyl cytosine. Backbone modifications are similarly known in the art, and include, chemical modifications to the phosphate linkage (e.g., phosphorodiamidate, phosphorothioate (PS), N3′phosphoramidate (NP), boranophosphate, 2′, 5′phosphodiester, amide-linked, phosphonoacetate (PACE), morpholino, peptide nucleic acid (PNA) and inverted linkages (5′-5′ and 3′-3′ linkages)) and sugar modifications (e.g., 2′-O-Me, UNA, LNA).

The oligonucleotides described herein may be synthesized using standard solid or solution phase synthesis techniques that are known in the art. In certain embodiments, the oligonucleotides are synthesized using solid-phase phosphoramidite chemistry (U.S. Pat. No. 6,773,885) with automated synthesizers. Chemical synthesis of nucleic acids allows for the production of various forms of the nucleic acids with modified linkages, chimeric compositions, and nonstandard bases or modifying groups attached in chosen places through the nucleic acid's entire length.

Certain embodiments of the invention encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

The term “complementary” as used herein refers to the broad concept of complementary base pairing between two nucleic acids aligned in an antisense position in relation to each other. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, at least about 60% and or at least about 80% of corresponding positions in each of the molecules are occupied by nucleotides which normally base pair with each other (e.g., A:T (A:U for RNA) and G:C nucleotide pairs).

The term “subject” as used herein refers to humans, higher non-human primates, rodents, domestic, cows, horses, pigs, sheep, dogs and cats. In one embodiment, the subject is a human.

The term “therapeutically effective amount,” in reference to treating a disease state/condition, refers to an amount of a therapeutic agent that is capable of having any detectable, positive effect on any symptom, aspect, or characteristics of a disease state/condition when administered as a single dose or in multiple doses. Such effect need not be absolute to be beneficial.

The terms “treat” and “treatment” refer to both therapeutic treatment and prophylactic or preventative measures, wherein the object is to prevent or decrease an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

### ASPECTS OF THE INVENTION

In certain embodiments, the present invention provides a DNA nanostructure nanorobot comprising:

a single stranded DNA scaffold strand of about 5,000 to 10,000 bases in length;

a plurality of staple strands of DNA, wherein each staple strands are about 20 to 40 bases in length, wherein each staple strand has a unique sequence and is hybridized to a specific position on the DNA scaffold strand, wherein the plurality of staple strands hybridized to the DNA scaffold form a sheet having a top surface and a bottom surface; and

one or more fastener strands of DNA, wherein the one or more fastener strands of DNA is capable of fastening the sheet into an origami structure.

In certain embodiments, the DNA nanostructure nanorobot further comprises one or more DNA targeting strands, wherein each targeting strand is operably linked to a targeting moiety.

In certain embodiments, the targeting moiety is an aptamer that specifically binds a target molecule.

In certain embodiments, the aptamer is specific for nucleolin.

In certain embodiments, the targeting strand comprises a domain for attaching to the single stranded DNA scaffold strand.

In certain embodiments, the DNA nanostructure nanorobot, further comprises DNA imaging strands, wherein each imaging strand is operably linked to an imaging agent.

In certain embodiments, the imaging agent is a fluorescent dye.

In certain embodiments, the sheet is a rectangle having four corners and it shaped into a tube-shape.

In certain embodiments, the dimension of the rectangular sheet is about 90 nm×about 60 nm×about 2 nm.

In certain embodiments, the one or more targeting strands are positioned at one or more corners of the rectangular sheet.

In certain embodiments, the tube-shaped origami structure has a diameter of about 19 nm.

In certain embodiments, each of the fastener stands of DNA comprise a first and a second strand of DNA.

In certain embodiments, the first and second strand of DNA form a Y-shaped structure.

In certain embodiments, the second strand of DNA comprises a domain partially complementary to the first strand.

In certain embodiments, the first and second strands hybridize to form a 14- to 16-base pair duplex.

In certain embodiments, the first strand of DNA comprises an aptamer that specifically binds a target molecule and a domain partially complementary to the second strand.

In certain embodiments, the aptamer specifically binds nucleolin.

In certain embodiments, the aptamer that specifically binds nucleolin is an F50 AS1411 aptamer sequence.

In certain embodiments, the oligonucleotide partially complementary to the aptamer comprises a Comp15 DNA sequence.

In certain embodiments, the first or second strand comprises a quencher moiety.

In certain embodiments, the other of the first or second strand comprises a fluorophore moiety.

In certain embodiments, the Y-shaped structure comprises:

5′-FITC-labeled F50 and 3′-BHQ1-labeled Comp15;

FITC-F50-48 and Comp15-48-Q;

FITC-F50-73 and Comp15-73-Q;

FITC-F50-97 and Comp15-97-Q;

FITC-F50-120 and Comp15-120-Q;

FITC-F50-144 and, Comp15-144-Q; or

FITC-F50-169 and Comp15-169-Q.

In certain embodiments, the nanorobot further comprises from one to four capture strands.

In certain embodiments, the one or more capture strand binds to a poly(A) region in the DNA scaffold strand.

In certain embodiments, the one or more capture strand is positioned on the top surface of the sheet.

In certain embodiments, the capture strand is positioned on the bottom surface sheet.

In certain embodiments, one or more capture strands is operably linked to a therapeutic agent.

In certain embodiments, the one or more capture strand comprises poly(T).

In certain embodiments, the one or more capture strand comprises an imaging agent.

In certain embodiments, the imaging agent is a fluorescent dye.

In certain embodiments, the therapeutic agent is a protein.

In certain embodiments, the therapeutic agent is thrombin.

In certain embodiments, the thrombin is conjugated to the functional strand of DNA by means of a sulfosuccinimidyl-4-(N-maleimidomethyl) cyclohexane-1-carboxylate (sulfo-SMCC) as a bifunctional crosslinker.

In certain embodiments, the nanorobot comprises from one to four thrombin molecules.

In certain embodiments, the nanorobot comprises four thrombin molecules.

In certain embodiments, the thrombin is operably linked to a fluorescent dye.

In certain embodiments, each staple strand is about 25 to 35 bases in length.

In certain embodiments, each staple strand is about 32 bases in length.

In certain embodiments, the present invention provides a DNA nanostructure nanorobot comprising:

a single stranded DNA scaffold strand comprising M13 phage DNA;

a plurality of staple strands 13-204 of DNA, wherein the plurality of staple strands hybridized to the DNA scaffold forms a rectangular sheet having a top surface and a bottom surface, and four corners;

at least six fastener strands of DNA, wherein each fastener strand of DNA is capable of fastening the rectangular sheet into a tube-shaped origami structure;

four DNA capture strands, wherein each capture strand is operably linked to a thrombin; and

at least four targeting strands, wherein each targeting strand is operably linked to an aptamer specific for nucleolin;

a plurality of imaging strands comprising extended ssDNA sequences that hybridized to fluorescent dye-labeled ssDNA.

In certain embodiments, the present invention provides a pharmaceutical composition comprising the DNA nanostructure nanorobot as described herein and a pharmaceutically acceptable carrier

In certain embodiments, the pharmaceutical composition further comprises at least one therapeutic agent.

In certain embodiments, the at least one therapeutic agent is a chemotherapeutic agent.

In certain embodiments, the present invention provides a method of treating a disease or disorder in a subject, comprising administering to the subject a therapeutically effective amount of the DNA nanostructure nanorobot or a composition as described herein.

In certain embodiments, the disease or disorder is cancer.

In certain embodiments, the cancer is breast cancer, ovarian cancer, melanoma or lung cancer.

In certain embodiments, the present invention provides a method of inhibiting tumor growth in a subject, comprising administering to the subject a therapeutically effective amount of the DNA nanostructure nanorobot or a composition as described herein.

In certain embodiments, the present invention provides a use of the DNA nanostructure nanorobot or a composition as described herein for the manufacture of a medicament for inducing a tumor necrosis response in a subject.

In certain embodiments, the present invention provides a use of the DNA nanostructure nanorobot or a composition as described herein for inducing a tumor necrosis response.

In certain embodiments, the present invention provides a use of the DNA nanostructure nanorobot or a composition as described herein for the manufacture of a medicament for treating a disease or disorder in a subject.

In certain embodiments, the present invention provides a use of the DNA nanostructure nanorobot or a composition as described herein for the prophylactic or therapeutic treatment a disease or disorder.

In certain embodiments, the present invention provides a kit comprising the DNA nanostructure nanorobot or a composition as described herein and instructions for administering the DNA nanostructure nanorobot/composition to a subject to induce an immune response or to treat a disease or disorder.

In certain embodiments, the kit further comprises at least one therapeutic agent.

The invention will now be illustrated by the following non-limiting Examples.

### Example 1

### Therapeutic DNA Nanorobot with on-Target Tumor Infarction for Cancer Therapy

DNA origami is a method that enables the rational design and production of DNA nanostructures with controlled size, shape and spatial addressability¹⁴⁻¹⁹, producing functional platforms for biological applications^{3, 5, 7, 20-23}. Inspired by these advances, a DNA nanorobotic system was constructed to protect thrombin until triggered only when localized in tumor vessels. This was accomplished by designing a method to create thrombin-DNA co-assembling nanostructures with multiple functional elements. Using the thrombin-loaded nanorobot, on-site tumor blood vessel infarction and targeted cancer treatment was demonstrated in vivo.

Results

Design and Synthesis of Thrombin-Loaded DNA Nanorobot

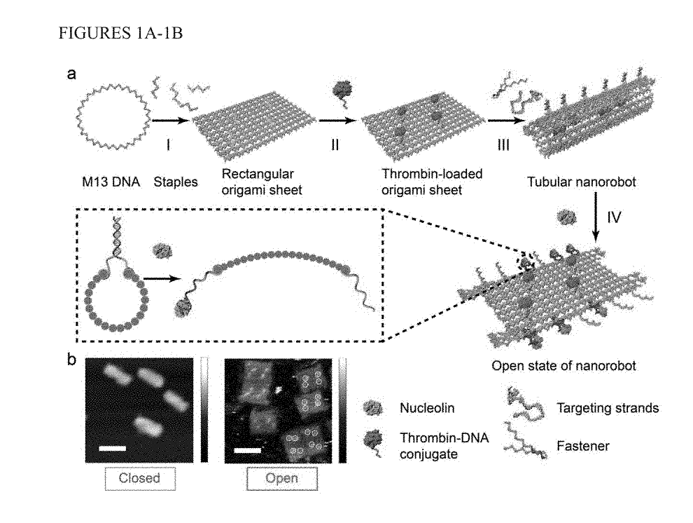

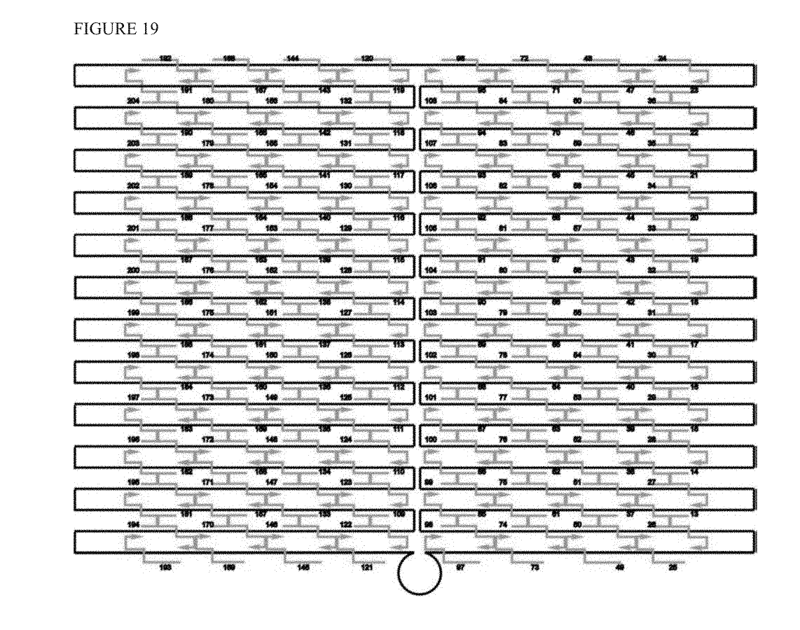

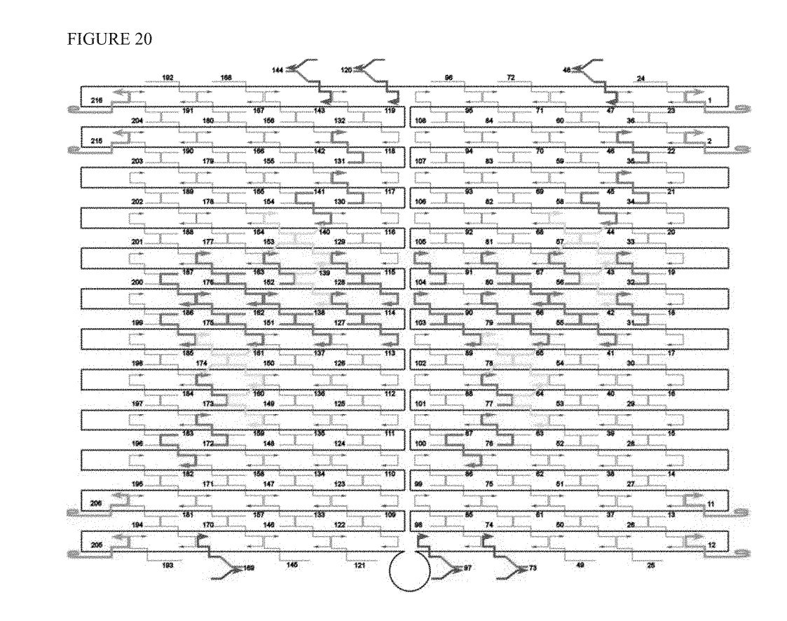

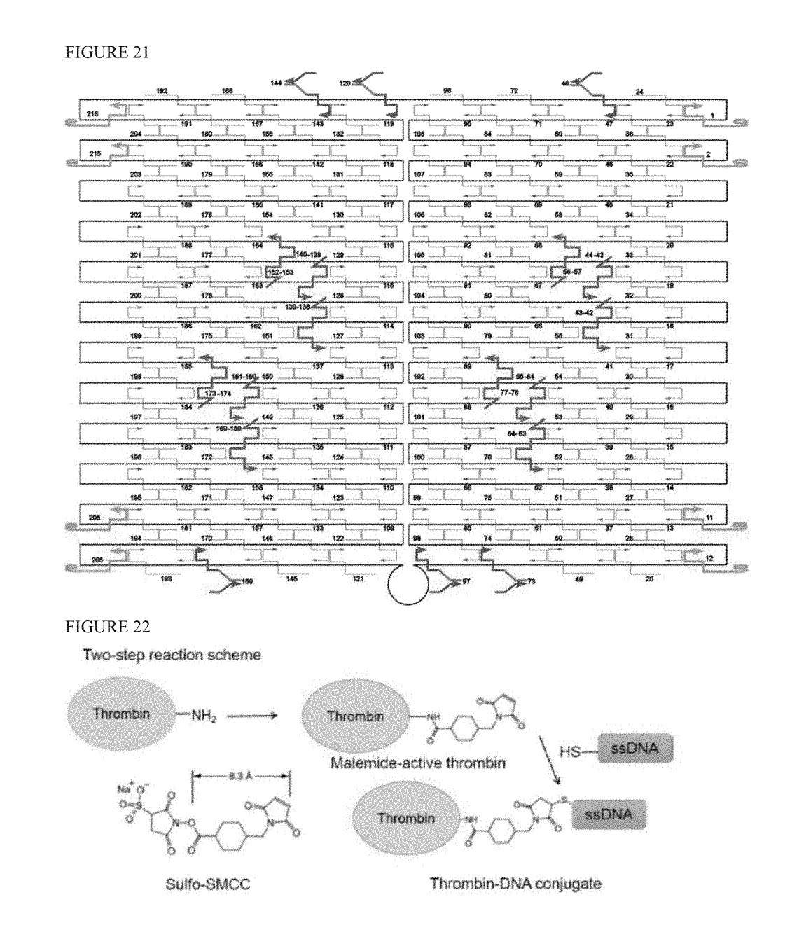

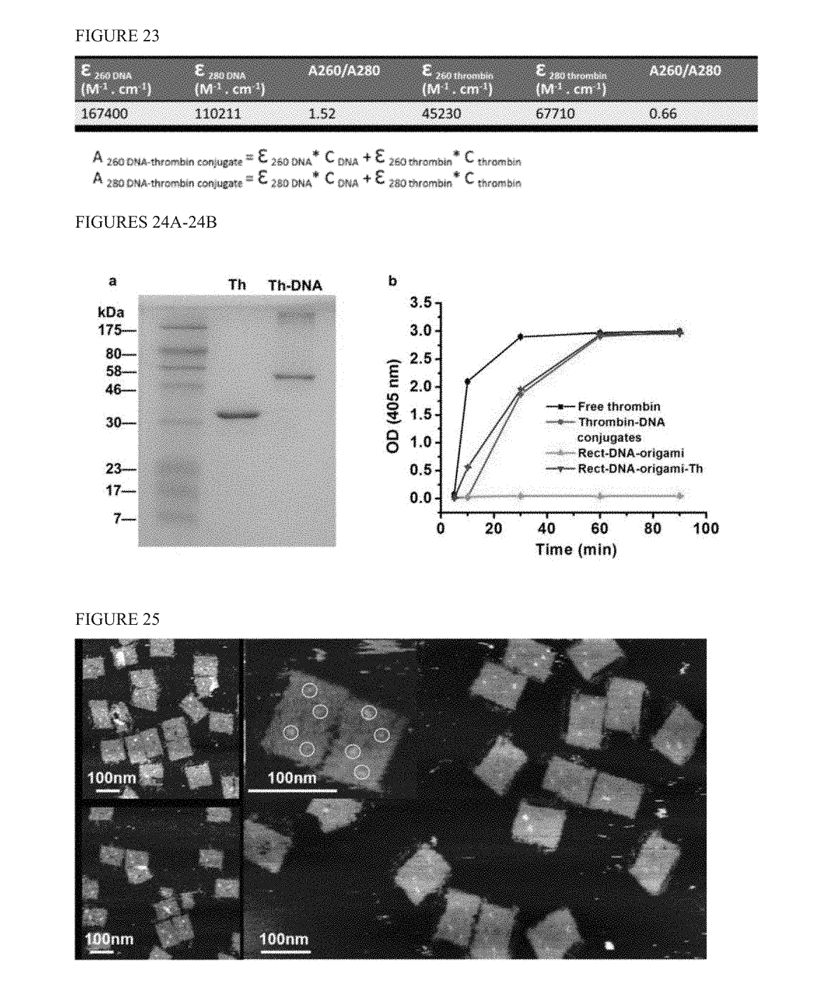

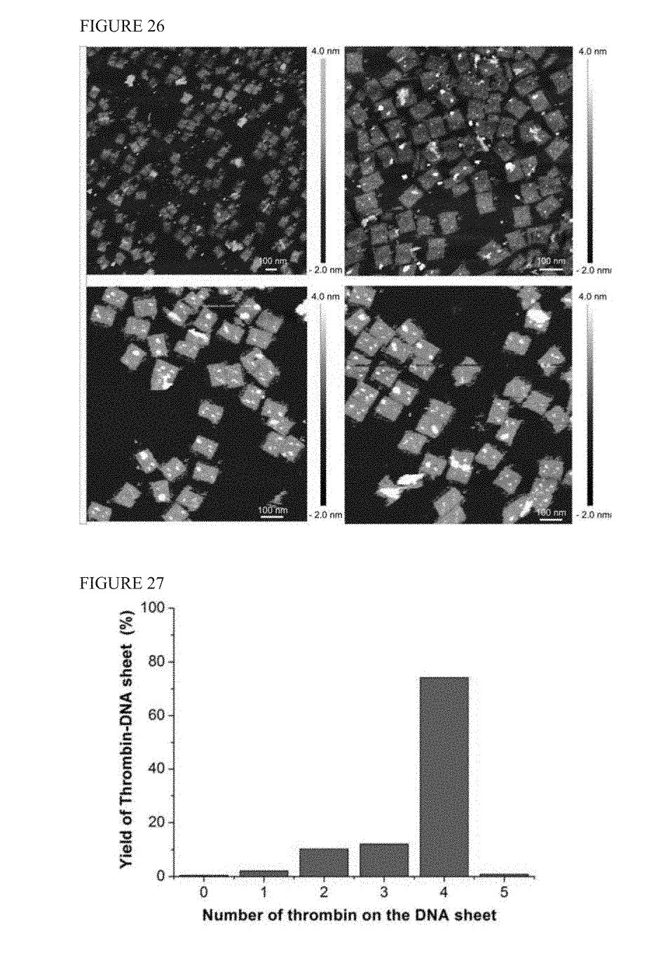

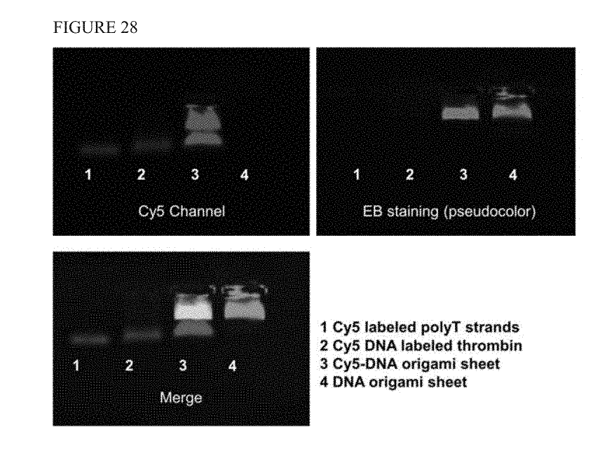

To deliver active thrombin solely to tumor sites in a highly controlled way, a DNA nanorobotic system was developed based on a self-assembled origami nanotube with multiple functional elements (FIG. 1a). A rectangular DNA origami sheet with the dimensions of 90 nm×60 nm×2 nm (FIG. 19) was first prepared by assembling a M13 bacteriophage genome DNA strand and multiple staple strands. For thrombin loading, capture strands with polyA sequences were extended at four designated locations on the surface of each DNA origami sheet (FIG. 20). Through rational design of the extension sites, the capture stands can be positioned either on the top or bottom surface of the sheets (FIG. 21). The thrombin-DNA conjugates were synthesized by attaching thiolated polyT oligonucleotides to thrombin molecules through a crosslinker sulfo-SMCC (sulfosuccinimidyl-4-[N-maleimidomethyl]cyclohexane-1-carboxylate) (FIGS. 22 and 23), and the conjugates were mixed with the DNA origami sheet. The extended polyA strands on the DNA sheet were then able to hybridize with the polyT on the conjugates (FIG. 24a), allowing thrombin molecules to be anchored on the surface of the DNA sheet (FIG. 1a). Biological activity assays showed that both thrombin-DNA conjugates and thrombin bound to the DNA origami sheet retained their catalytic activity (FIG. 24b). Atomic force microscopy images demonstrated that more than 70% of the DNA sheets contained four bound thrombin molecules (FIGS. 25-27). Using a Cy5.5-labeling approach for thrombin quantification, the average number of thrombin on each DNA origami sheet was calculated to be 3.8±0.4 (FIG. 28).

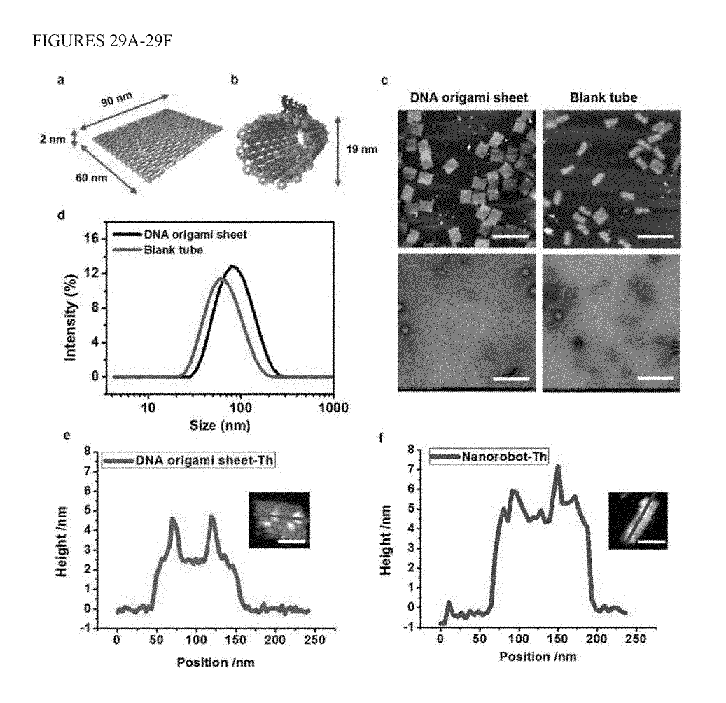

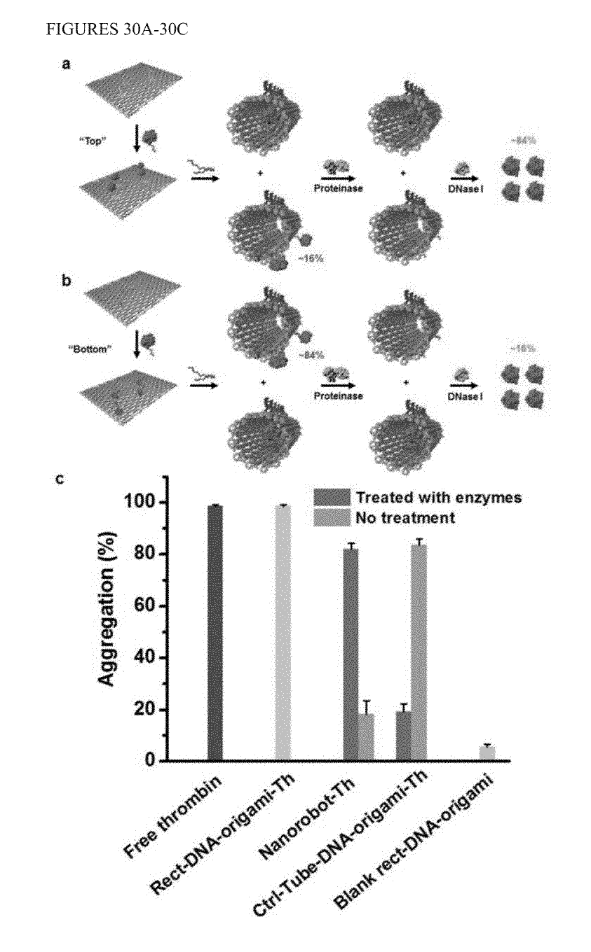

The present hollow tube-shaped DNA nanorobot, with a diameter of ˜19 nm and a length of 90 nm (FIGS. 1a and 29A-29F), was next formed by fastening the thrombin bound DNA origami sheet along the long sides (FIGS. 1a and 20). The four thrombin molecules, attached to the inner surface of the tubular nanorobot (FIG. 30A-30C), became thus shielded from circulating platelets and plasma fibrinogen. By hybridization of pre-designed fastener strands containing DNA aptamers (AS1411)²⁴, the tube-shaped DNA nanorobot was non-covalently closed along a defined seam (FIGS. 1a and 29A-29F). The closed and open states of the DNA nanorobot were confirmed by atomic force microscopy (FIG. 1b). The four bright spots on the surface of origami sheet represent the raised height produced by the thrombin cargo.

DNA Nanorobot-Triggered Activation and Endothelial Cell Targeting In Vitro

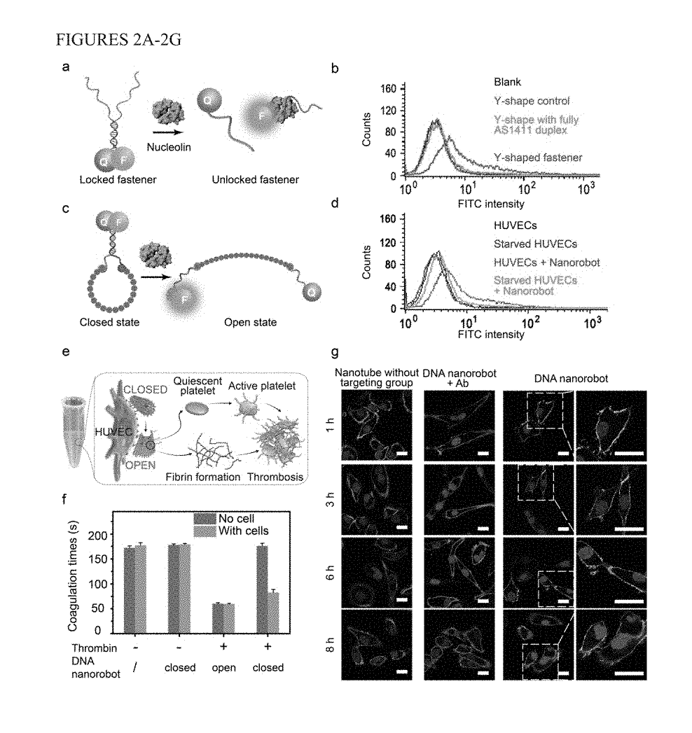

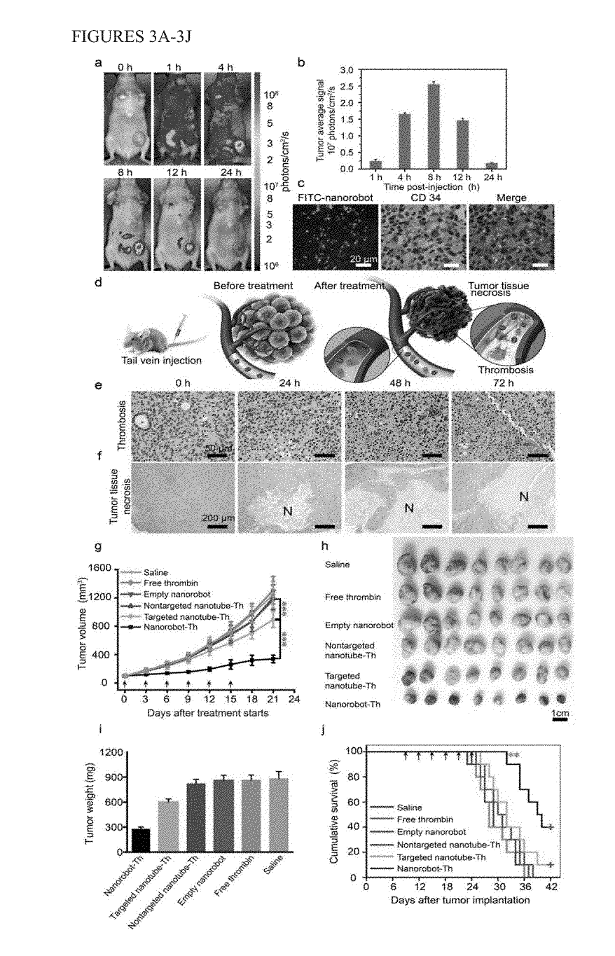

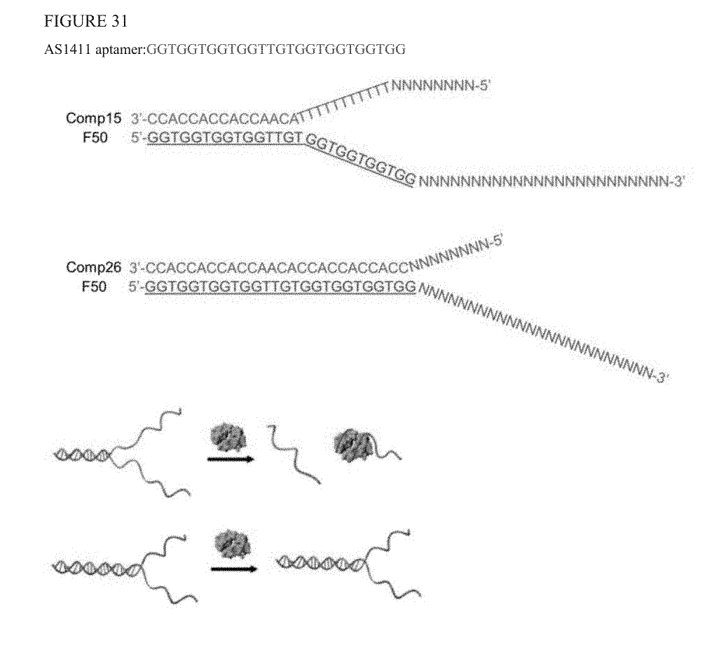

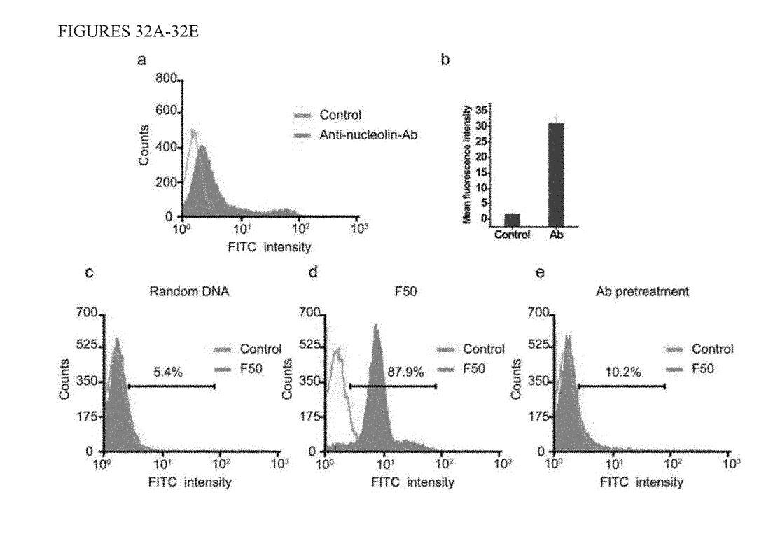

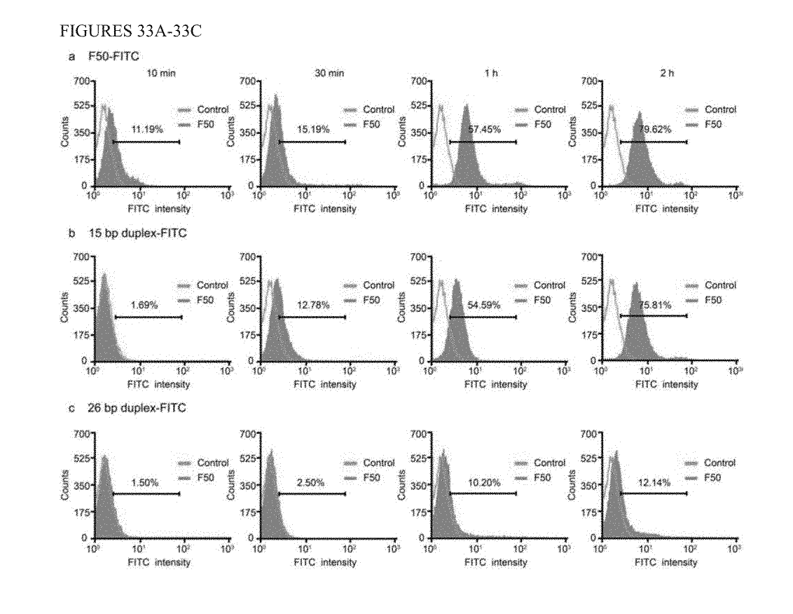

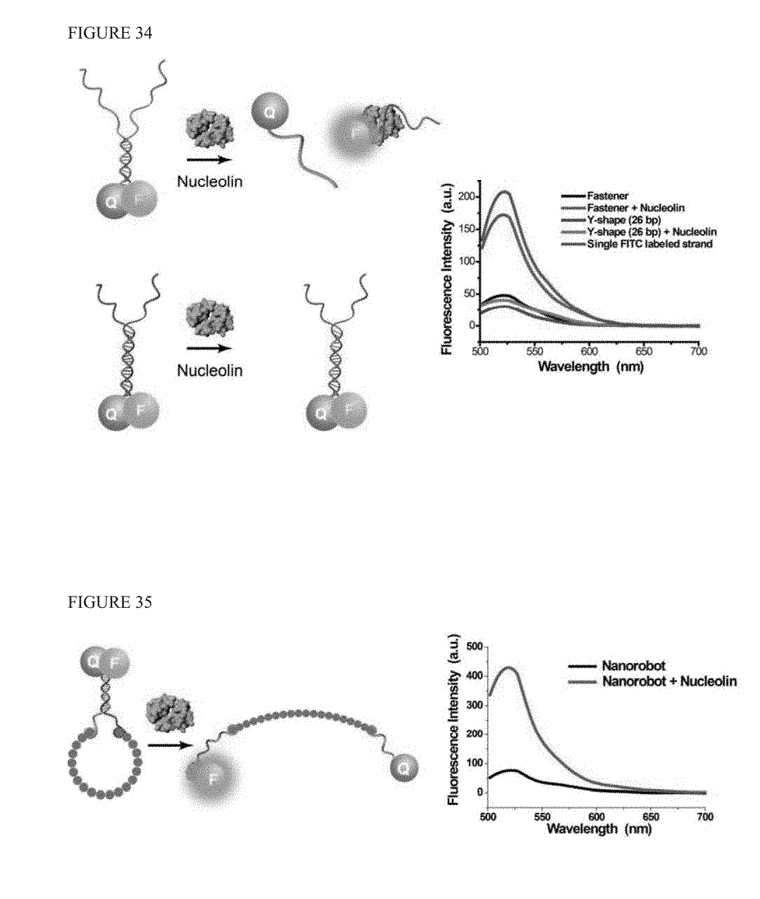

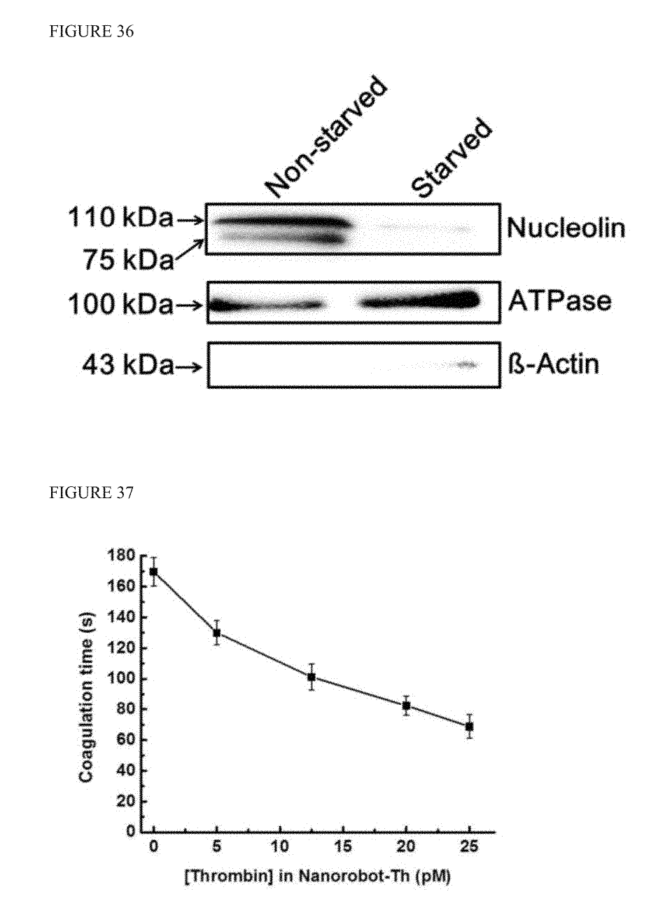

It was hypothesized that when the fastener strands recognize their targets, e.g., nucleolin proteins selectively expressed on the surface of actively proliferating tumor vascular endothelial cells²⁵, the hybridized duplexes would dissociate to induce reconfiguration of the nanorobot to expose the contained cargo. To test this hypothesis, flow cytometry was used to examine the nucleolin recognition and dissociation of the DNA fastener strands. The Y-shaped fastener structures (FIG. 31), each containing a 15-base pair duplex, were composed of two DNA strands: F50, which contains AS1411 aptamer sequences that are able to specifically bind to nucleolin expressed on the surface of cultured human umbilical vein endothelial cells (HUVECs; FIG. 32A-32E), and Comp15, a strand partially complementary to the AS1411 sequence of F50. As with free F50, the Y-shape structure containing the 15-bp duplex was able to effectively bind to HUVECs over time (FIG. 33A-33E), demonstrating the recognition property of the fastener strands for surface nucleolin. To determine whether the fasteners can dissociate after binding to recombinant nucleolin or nucleolin-positive HUVECs, we prepared dye-labeled fasteners (5′-fluorescence-labeled F50 and 3′-quencher-labeled Comp15; FIG. 2a and FIG. 34) serving as switchable fluorescent beacons. These beacons emit only a weak fluorescence when hybridized. Upon recognizing recombinant nucleolin or that on the surface of HUVECs, the fasteners activate: fluorophore-labeled F50 strands switch to the G-quadruplex state²⁶ and bind to nucleolin, while the quencher-labeled Comp15 is released, resulting in a high fluorescence intensity triggered by binding to the protein in solution (FIG. 34) or on the HUVECs (FIG. 2b). For comparison, the other two Y-shaped structures (fully complementary 26-bp duplex of AS1411 sequences or controls lacking any AS1411 sequence) were not activated by HUVECs (FIG. 2b). A similar experiment was performed to demonstrate that the binding of nucleolin can open the DNA nanorobots. Six pairs of Y-shaped fastener structures consisting of 5′-FITC-labeled F50 and 3′-BHQ1-labeled Comp15 were used to close the DNA nanorobot. With the DNA nanorobot in the “fastened” state, the FITC fluorescence is quenched by BHQ1. Upon opening, a strong increase in fluorescence intensity is expected (FIG. 2c and FIG. 35, left panel). Fluorophore-quencher pair-labeled nanorobot was treated with recombinant nucleolin and a significant increase in fluorescence intensity was observed, indicating the opening of the nanorobot. We also used nucleolin-positive HUVECs to induce the opening of the fastened DNA nanorobot (FIG. 35). After recognizing the nucleolin expressed on the surface of HUVECs, the reconfiguration of the DNA nanorobot was triggered, resulting in enhanced fluorescence intensity. In contrast, with starved HUVECs, whose surface nucleolin expression is down-regulated (FIG. 36), the DNA nanorobot did not open (FIG. 2d).

Next, in vitro blood coagulation was investigated to determine whether reconfiguration of the nanorobot occurs in response to the target protein. When mouse plasma was mixed with the thrombin-loaded nanorobot (nanorobot-Th) and HUVECs, it coagulated rapidly, with a fibrin formation time of 82 s, as compared to 176 s when cells were absent (FIG. 2e-f). This enhanced coagulation was comparable to the fastener-free, thrombin-loaded DNA origami sheet, indicating a cell-induced opening of the nanotubes. No effect on coagulation time was seen when plasma was added to cells alone, or to cells incubated with empty DNA nanorobot. Further, it was found that there was a positive relationship between the rate of plasma coagulation and the concentration of nanorobot-Th added (FIG. 37). These data demonstrate that the DNA nanorobot is able to change its structure in response to target cells, and that the exposed thrombin payload can induce coagulation.

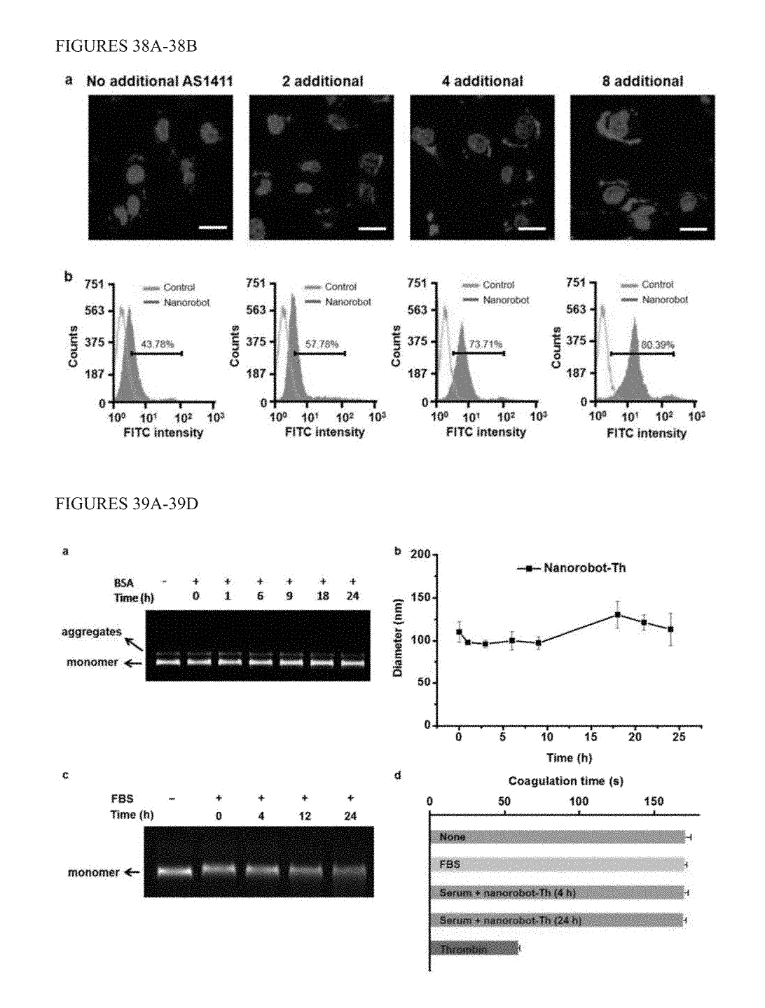

The ability of the DNA nanorobot to target surface nucleolin-positive cells was next examined. To ensure a maximal targeting effect in vitro, we decorated the staple strands of the origami structures with additional targeting aptamer sequences at both ends of the tubes (FIGS. 1a and 20). To visualize the endothelial cell targeting, we modified the staple strands of the central region of origami structure to contain a single strand extension (imaging strand) that can hybridize to Alexa 594-labeled oligonucleotides (FIG. 20). The results of confocal microscopy and flow cytometry assays with these modified nanorobots suggest that the 8 additional AS1411 strands at both ends (four for each end) elicited a maximal binding to cultured HUVECs (FIG. 38A-38B). Time course data further reveal a rapid association of such nanorobots to HUVECs, with maximal binding attained within 1 h and a cell surface dwell time of at least 6 h (FIG. 2g). In contrast, the bare nanotubes rolled without the targeting aptamer strands (referred to as “nontargeted nanotubes”) did not bind to the surface of HUVECs. The binding of nanorobot was dependent upon cell surface nucleolin as the presence of an anti-nucleolin antibody abolished the binding. Treatment of HUVECs with free AS1411 was used as a positive control for binding. In addition, nanorobot-Th was stable over a 24 h period in the presence of bovine serum albumin (BSA) or fetal bovine serum (FBS) (FIG. 39A-39D), suggesting a potential high stability in blood circulation.

In Vivo Tumor Targeting and Biodistribution of DNA Nanorobots

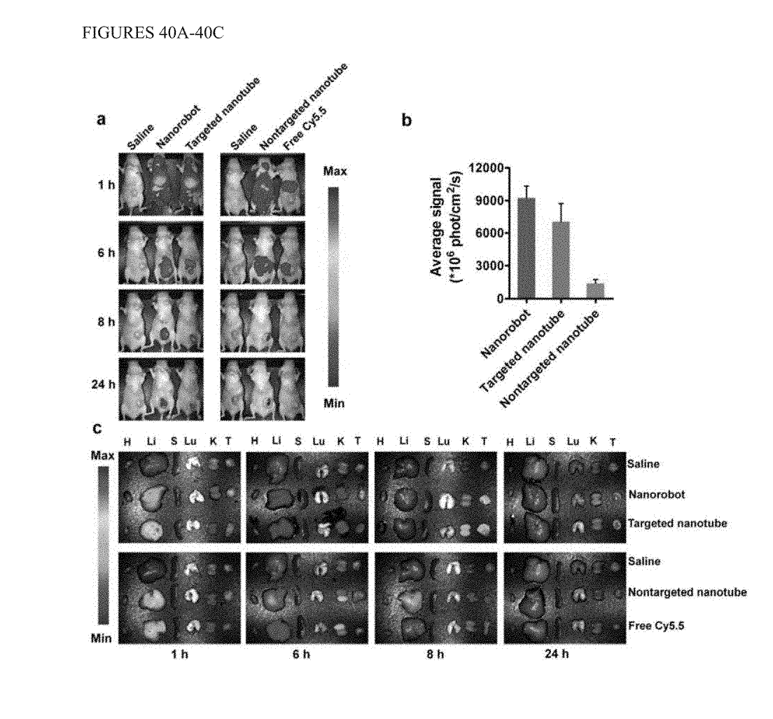

Fluorescence imaging was performed on orthotopic tumor-bearing mice to investigate tumor vessel targeting of the nanorobot in vivo. Human breast cancer cells (MDA-MB-231) were injected into the mammary fat pads of BALB/c nude mice for the establishment of a tumor model. Mice displaying tumor volumes around 100 mm³ were intravenously injected (i.v.) with Cy5.5-labled DNA nanorobots. The nanorobots progressively accumulated in the tumor, reaching a maximal accumulation at 8 h post-injection (FIG. 3a,b). Substantial differences were observed between the targeted nanorobots and nontargeted nanotubes in the tumor signal intensities (FIG. 40a-40b), whereas the ex vivo fluorescence signals from other major organs were similar (FIG. 40c). Quantitative analysis revealed that the targeted nanorobots accumulated in the tumor ˜7 times more efficiently than the nontargeted nanotubes 8 h after injection (FIG. 40b). This result indicates that the aptamer-conjugated nanorobots were able to target tumors in vivo. To further determine whether the nanorobot indeed targets the nucleolin-positive tumor endothelium, tumor sections were stained for the endothelial cell marker CD34 after administration of FITC-labeled nanorobots. The nanorobots effectively bound to the tumor vascular endothelium, as evidenced by co-localization with CD34 (FIG. 3c).

The time-dependent organ distribution and clearance of DNA nanorobots and other controls after intravenous injection was also examined (FIG. 40c). DNA nanostructures strongly accumulated in the liver shortly after injection (1 h), presumably captured by the mononuclear phagocyte system. In time, the intensity of the liver fluorescence decreased, implying that the nanostructures not entrapped by the tumor may be degraded following liver phagocytosis, as generally suggested for nanomaterials of similar size²⁷. At 24 h post-injection, little fluorescence was observable in the mice and major organs, demonstrating the successful clearance or degradation of the material from the body.

Nanorobots Induce On-Site Tumor Vessel Occlusion and Necrosis

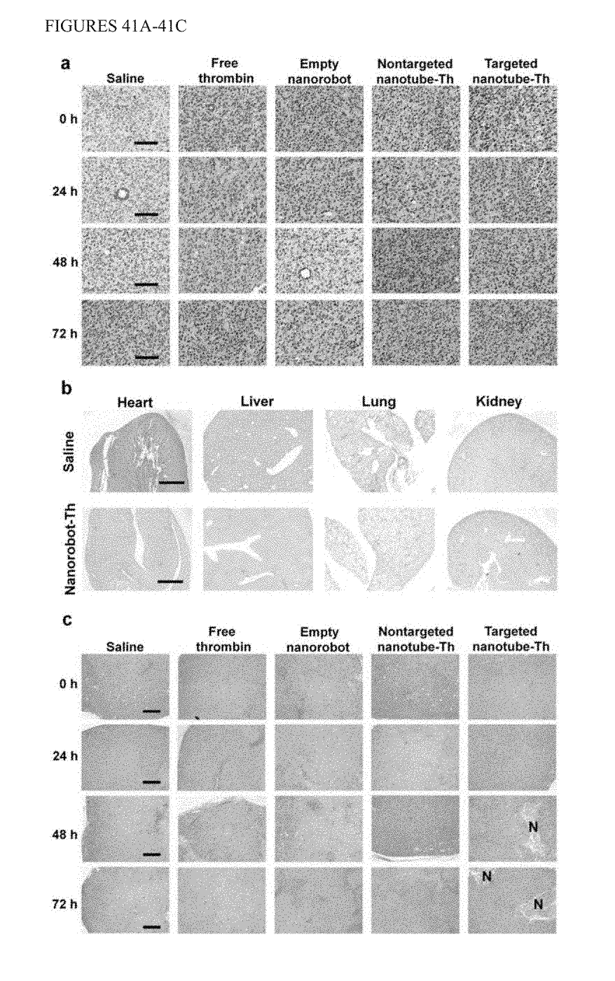

After targeting delivery to tumor-associated blood vessels, the tubular shaped nanorobot undergoes a structural reconfiguration, triggered by nucleolin-mediated unfastening, to expose the loaded thrombin. The thrombin proteins localized to tumor vessels induce thrombosis by activating platelets and inducing the generation of fibrin strands, resulting in vessel infarction and tumor necrosis (FIG. 3d). To evaluate the triggered activation and exposure of the molecular payload, the tumor vessels were assessed for targeted thrombosis after administration of nanorobot-Th (FIG. 3e). Blood vessels in the tumor region were occluded within 24 h, as demonstrated by the detection of widespread platelet aggregates using an antibody specific for CD41 on the activated platelet surface. After 48 h, there was advanced thrombosis, and by 72 h, dense thrombi in all tumor vessels were observed. In contrast, there was no visible thrombosis in the tumor vessels of mice after administration of saline or equivalent doses of free thrombin, targeted empty nanorobot or nontargeted thrombin-loaded nanotube (nanotube-Th) for up to 72 h (FIG. 41a). A weak thrombotic activity was observed with targeted nanotube-Th (FIG. 41a, 72 h) that has a similar tumor targeting ability as nanorobot-Th (FIG. 40A-40C), but was unable to open after binding to nucleolin. It is possible that a passive degradation of targeted nanotubes within tumors results in exposure of a portion of the active thrombin to induce thrombosis. Neither thrombi nor histological abnormalities were found in the heart, liver, lung or kidney from the tumor-bearing mice 72 h after administration of nanorobot-Th (FIG. 41b), verifying that thrombosis was specific to the tumor vasculature. As expected, haematoxylin and eosin (H&E) staining revealed advanced tumor necrosis over time in the nanorobot-Th treated group (FIG. 3f), and limited necrosis was also found in the targeted nanotube-Th treated group (FIG. 41c), while no necrosis was observed in any other group.

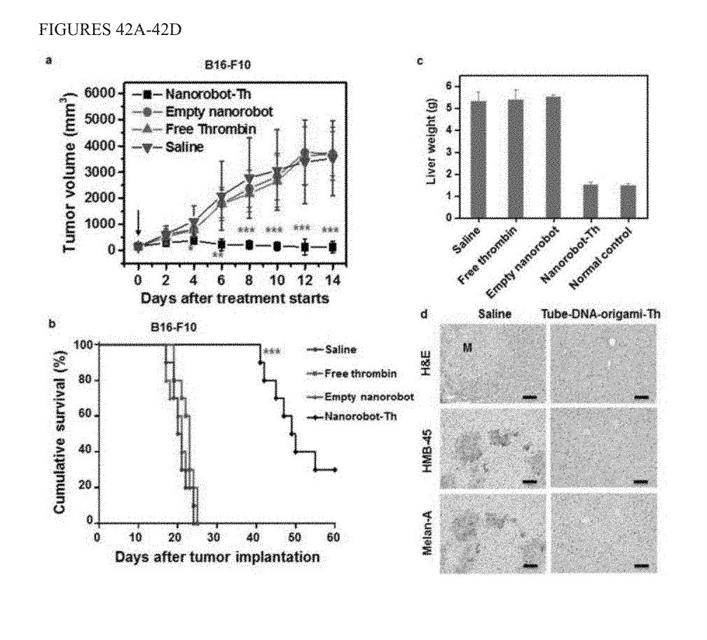

To investigate the therapeutic potential of nanorobot-Th, MDA-MB231 tumor-bearing nude mice were randomly sorted and each group was treated through tail vein injection as follows: saline, free thrombin, targeted empty nanorobot, nontargeted nanotube-Th, targeted nanotube-Th or nanorobot-Th. Injections were carried out 6 times at intervals of 3 days. Mice treated with saline, free thrombin, empty nanorobot or nontargeted nanotube-Th formed fast growing tumors (FIG. 3g-i). In contrast, tumors grew significantly more slowly in mice treated with nanorobot-Th, indicating a therapeutic effect in inhibiting tumor growth. This correlated with a substantial increase in animal survival (FIG. 3j); the median survival time for mice treated with saline was 29 days, and treatment with free thrombin, empty nanorobots or nontargeted nanotube-Th had no significant effect on this time. Importantly, nanorobot-Th treatment increased this survival to 39 days (Kaplan-Meier curves, p=0.0042). Tumors in mice receiving targeted nanotube-Th grew slightly slower than did tumors in mice receiving saline, free thrombin, empty nanorobots or nontargeted nanotube-Th (FIG. 3g-i), and treatment with targeted nanotube-Th slightly increased animal survival to 32 days (FIG. 3j). The weak inhibitory effect of targeted nanotube-Th on tumor growth presumably derives from its modest thrombogenic activity in tumor vessels, as evidenced in tumor sections (FIG. 41a). The nanorobot-Th proved to be even more effective in a B16-F10 melanoma mouse model (FIG. 42a, 42b), in which 3 out of 8 of the mice receiving nanorobot-Th showed complete regression of the tumors. The median survival time of the mice was extended from 20.5 to 45 days compared to the saline group. This higher efficacy was likely a consequence of the higher grade of vascularization of melanoma tumors. Nanorobot-Th treatment effectively prevented the occurrence of melanoma metastases in the liver (FIG. 42c, 42d). This may be attributed to the inhibition of primary tumor progression or to the regression of vascularized metastases.

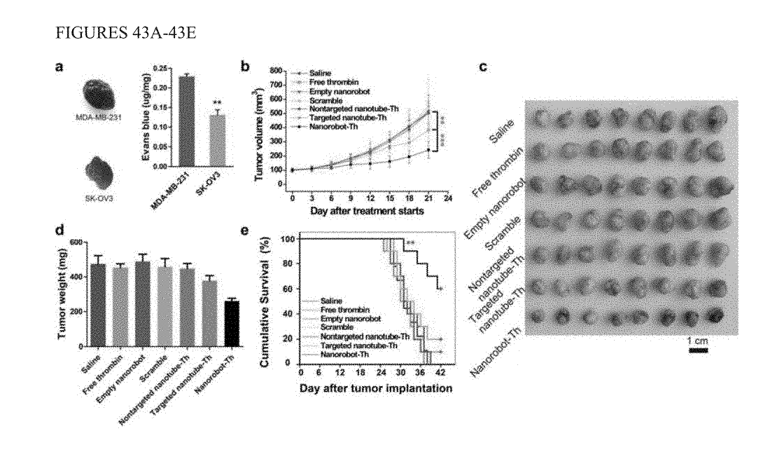

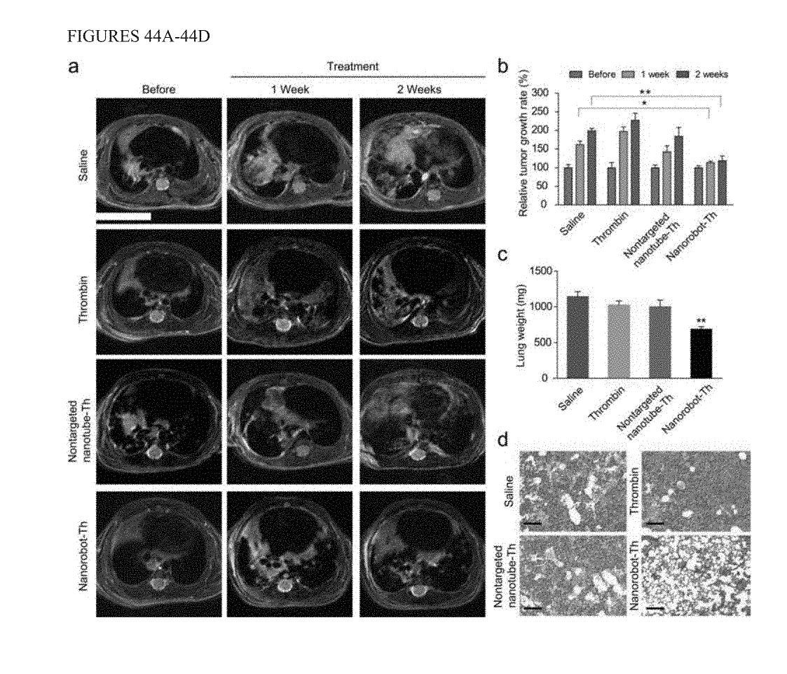

In order to further explore the role of targeted delivery, its antitumor activity in less vascularized tumors was investigated. Mice bearing subcutaneous xenografts of human ovarian cancer cells SK-OV3, reported to be poorly vascularized²⁸ and exhibited relatively low permeability and retention for Evans blue (FIG. 43a), showed significantly restrained tumor growth after treatment with nanorobot-Th, compared to when treated with saline, free thrombin or other control nanostructures (FIG. 43b-43d). Nanorobot-Th treatment also significantly prolonged the survival of animals (p=0.03; FIG. 43e). Although in this case the inhibitory efficiency of the DNA nanorobot was not as remarkable as in the melanomas, these results still implied that limited tumor permeability did not prevent the system to exert antitumor activities. Moreover, encouraging therapeutic results were also observed in a doxycycline-inducible KRAS mutation lung cancer model, generated using transgenic TetO-KRAS^{G12D} mice²⁹, faithfully recapitulating the clinical course of lung cancer patients. Magnetic resonance imaging (MRI) was used to assess tumor extent before and after 1- and 2-week period of treatment with nanorobot-Th once every three days. TetO-KRAS^{G12D} mutant mice showed a clear response to nanorobot-T with a significant inhibition in tumor progression during 2 weeks of observation, as assessed by MRI (FIG. 44a, 44b) and a significant reduction in wet lung weight (FIG. 44c). H&E staining revealed that tumor regions were full of intratumoral spaces after 2-week treatment of nanorobot-Th (FIG. 44d), suggesting shrinkage of tumor tissues. H&E stained sections also showed thickening of alveolar wall and fibrosis, strongly arguing remodeling of the tumor tissues into normal lung tissues. In contrast, the mice treated with saline, thrombin, and nontargeting nanotube-Th displayed rapid tumor growth, with an approximately 100% increase in tumor growth during this period of treatment. Collectively, these data reveal that our targeted thrombin delivery system based on a reconfigurable DNA nanostructure exhibits promising in vivo antitumor efficacy, and is applicable for tumors of diverse vascularization level.

Safety Assessment for Thrombin-Loaded DNA Nanorobots

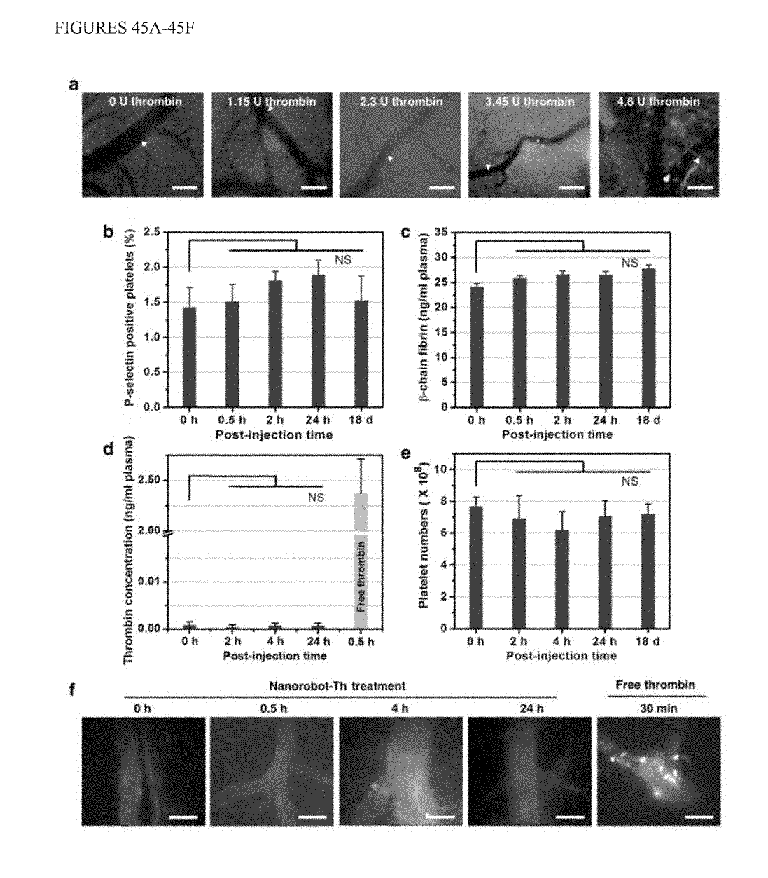

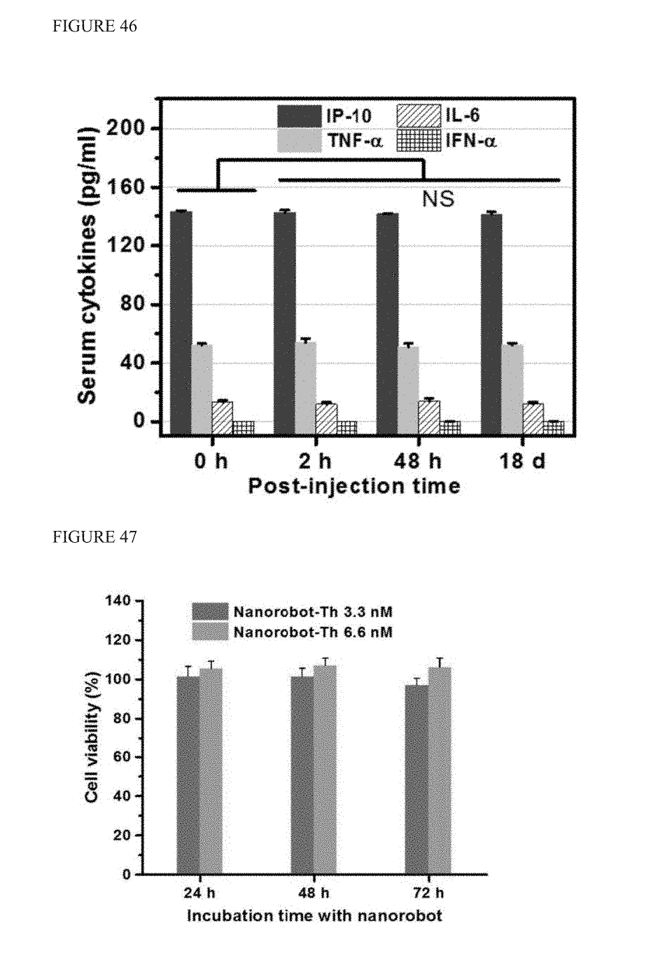

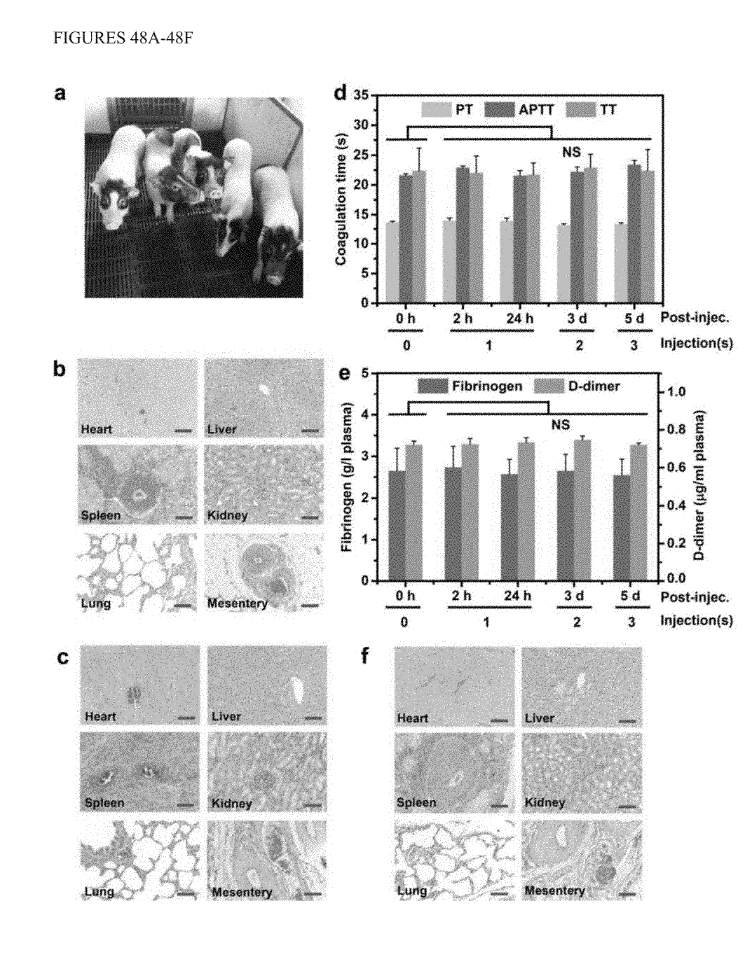

Finally, comprehensive in vitro and in vivo safety assessments of the DNA nanorobotic system were carried out. It was first found that doses of free thrombin equivalent to those used in vivo in the nanorobot system elicited no observable thrombi in the cerebral microcirculation in non-tumor bearing healthy mice. However, transient, reversible microthrombi were apparent in much higher concentrations (FIG. 43a). As for the DNA nanorobots themselves, no significant changes were observed in blood coagulation parameters (including platelet activity, plasma thrombin and fibrin concentrations and circulating platelet numbers) after treatment with nanorobot-Th in the non-tumor bearing mice compared to the control group (FIG. 43b-43e). There were also no observable cerebral microthrombi in mice 24 h after injection of nanorobot-Th (FIG. 43f). In addition, nanorobot-Th treatment had no significant impact on cytokine levels (IL-6, IP-10, TNF-α and IFN-α) (FIG. 44A-44D), suggesting the DNA nanorobot system is immunologically inert, which is in accordance with previous observations^{6,22,30,31}. The nanorobot-Th also did not cause any observable cytotoxicity in standard CCK-8 cell proliferation assays (FIG. 45A-45F).

The safety of the DNA nanorobots was further characterized in normal Bama miniature pigs, which exhibit high similarity to humans in anatomy and physiology³². Intravenous injections of free thrombin at 150 U/pig, a dose equivalent to the accumulated therapeutic dose of thrombin in the mouse model treated with nanorobot, had no effect on blood coagulation parameters and did not induce thrombosis in major organs (Fig. S28a-c). However, activated partial thromboplastin time (APTT) prolongation occurred but was still under 43 s after single or multiple injections of thrombin at up to 350 U/pig; D-dimer content also increased after injection of this dose, but disappeared with a final measurement of normal levels 3 d after the last treatment, indicating a transient and reversible blood coagulation system activation with no visible thrombotic formation in major tissues. Treatment with nanorobot-Th system did not lead to any significant variations in the blood coagulation parameters (Fig. S28d,e) or histological morphology (Fig. S28f) when compared to the control group, demonstrating that the nanorobot-Th is decidedly safe in the normal tissues of large animals.

### DISCUSSION

Directed coagulation of tumor vasculature has enormous potential to eradicate solid tumors, yet specifically targeting tumor blood supply via systemic therapeutic agent administration appears an impossible mission. Although intra-arterial embolization, such as transhepatic arterial embolization (TAE) and transhepatic arterial chemoembolization (TACE), is routine practice for the effective treatment of a variety of hepatic malignancies, assisted by image-guided therapy^{33, 34}, vascular occlusion-based cancer therapies remain challenging due to the lack of safe and effective vessel occluding agents administered systemically. A novel nanorobotic system was developed for the intelligent delivery of therapeutic thrombin in vivo to tumor-associated blood vessels, to elicit highly efficient blockage of tumor blood supply and inhibition of tumor growth. With tumor-targeted delivery, recognition of tumor microenvironmental signals, triggered nanostructural changes and payload exposure, the thrombin delivery DNA nanorobot constitutes a major advance in the application of DNA nanotechnology for cancer therapy. In a melanoma mouse model, the nanorobot not only affected the primary tumor, but also prevented the formation of metastasis, showing promising therapeutic potential.

Methods

Materials and Reagents.

Oligonucleotides (origami staple strands and functional strands) were purchased from Invitrogen (Shanghai, China) and used without further purification. The dye labeled DNA strands were further purified for use by denaturing polyacrylamide gel electrophoresis (PAGE). For flow cytometry and immunohistochemistry, the following antibodies³⁵⁻³⁶ were used for nucleolin (Sigma-Aldrich, St. Louis, Mo., catalog No. N2662); mouse platelet CD41 (BD Pharmingen, San Diego, Calif., catalog No. 553847); P-selectin (BD Pharmingen, San Diego, Calif., catalog No. 553744); CD34 (Sigma-Aldrich, St. Louis, Mo., catalog No. WH0000947M1); fibrin IIβ chain (Accurate Chemical & Scientific, Westbury, N.Y., catalog No. NYBT2G1P); thrombin (MyBioSource, San Diego, Calif., catalog No. MBS2001306); FITC-conjugated goat anti-rabbit IgG (BD Pharmingen, San Diego, Calif., catalog No. 554020); FITC-conjugated goat anti-mouse IgG (BD Pharmingen, San Diego, Calif., catalog No. 5540001) and Alexa Fluor 488 AffiniPure Donkey anti-rat IgG (Yeasen Biotech, Shanghai, China, catalog No. 34406ES60). Cell culture media, fetal bovine serum, hemacytometers, and cell culture supplies were purchased from Fisher Scientific. Tris-base, acetic acid, sodium chloride, ethylenediaminetetraacetic acid disodium salt dehydrate and other common chemical reagents were purchased from Sigma-Aldrich.

DNA origami design details. Production of M13 bacteriophage single-stranded DNA was according to Douglas et al.'s methods³⁷. In detail, JM109 E. coli were cultured overnight in LB medium, 5 ml were diluted in 2×YT medium with 5 mM MgCl₂ and placed in a 37° C. shaker. When the optical density (OD 600) reached 0.5, p7249 M13 phages were mixed with the bacteria, and the culture incubated in a 37° C. shaker. After a 5 h incubation, the culture was collected and centrifuged at 6,000 g for 30 min to remove the bacteria pellets. NaCl (30 g/l) and PEG (40 g/l) were added to the supernatant containing phages and the mixture was incubated on ice for 1 h. After 30 min centrifugation at 10,000 g, the phage pellet was collected and suspended in Tris-Cl (10 mM, pH 8.5). After adding 0.2 M NaOH and 1% SDS, the phage solution was mixed and incubated at 25° C. for 3 min. After the addition of potassium acetate (3 M, pH5.5) for neutralization, the solution was incubated on ice for 10 min and centrifuged (12,000 g, 30 min). The ssDNA (7249 nt) containing supernatant was collected and precipitated in ethanol (70%) on ice for 2 h. After centrifuging at 12,000 g for 30 min, the DNA pellet was collected and washed in ethanol (70%) and then resuspended in Tris-Cl (10 mM, pH 8.5). The concentrations of ssDNA were determined by UV-Vis spectrometry (Shimadzu Corp. Kyoto, Japan).

The original design of the rectangular DNA origami structure shown in FIG. 19 was established by Rothemund¹⁵. This design was used as the blank template in this work and functional strands were used to replace the original strands at the corresponding positions. The position and sequences of the functional staple strands are shown in different colors in FIGS. 19-21.

Rectangular DNA Nanosheets.

Rectangular DNA nanostructures were made by mixing a long single stranded scaffold strand (7249 DNA derived from M13 bacteriophage) with short strands (including staple strands and functional strands) in 1×TAE-Mg buffer (40 mM Tris, 20 mM acetic acid, pH 8.0, 2 mM EDTA, 12.5 mM Mg(CH₃COOH)₂). The final concentrations of scaffold DNA and basic staple strands were 10 nM and 80 nM, respectively. The mixture was heated to 65° C., then annealed by cooling to 25° C. at a rate of 10 min/° C. using an Eppendorf thermal cycler (Eppendorf Mastercycler ep Gradient S, Hamburg, Germany). Unless otherwise stated, all DNA strands or aptamers were purchased from the oligonucleotide synthesis service of Invitrogen (Invitrogen, Carlsbad, Calif.).

The resulting rectangular DNA origami sheets were separated from excess staple strands using Amicon Ultra-0.5 ml 100 kD centrifugal filters (Millipore Corporation, Bedford, USA). The initial filtration was performed by adding 350 μl 1×TAE-Mg buffer to 100 μl DNA sheets, and centrifuging for 3 min at 2,075 g. Then two wash steps were performed by adding 350 μl 1×TAE-Mg buffer and centrifuging for 3 min each at 2,075 g. The remaining solution was collected and characterized using 1% agarose gel electrophoresis and atomic force microscopy (AFM).

Preparation of Thrombin-DNA Conjugates and Thrombin Activity Analysis.

Thrombin-DNA conjugates were prepared using sulfosuccinimidyl-4-(N-maleimidomethyl) cyclohexane-1-carboxylate (sulfo-SMCC, Sigma-Aldrich, St. Louis, Mo.) as a bifunctional crosslinker between thrombin and DNA. In a typical synthesis, 200 μl 3 μM thrombin (Sigma-Aldrich, St. Louis, Mo., catalog No. T4648) was mixed with 9 μl 15 mM sulfo-SMCC at room temperature for 1 h. Excess sulfo-SMCC was removed using an Amicon Ultra-0.5 ml 30 kD centrifugal filter and washing three times by centrifugation. The initial washing was conducted by adding 250 μl PBS to 200 μl of the mixture of sulfo-SMCC and thrombin, and centrifuging for 3 min at 5,534 g. The subsequent two washing steps were performed by adding 350 μl PBS and centrifuging for 3 min at 5,534 g. The residual solution was collected and a 15-fold excess of thiolated polyT DNA was added. The mixture was incubated at 4° C. overnight and the final thrombin-DNA conjugates were purified using 30 kD centrifugal filters. Conjugation was verified by 4-12% SDS-polyacrylamide gel electrophoresis.

Thrombin activity was assayed using tosyl-glycyl-prolyl-arginine-4-nitroanilide acetate (chromozym TH) as a substrate, following the manufacturer's protocol (Boehringer Mannheim, Indianapolis, Ind.). In brief, reactions were conducted at 37° C. for 5 min, 10 min, 30 min, 1 h or 1.5 h, with free thrombin as a positive control and a blank DNA sheet as a negative control. Thrombin-DNA conjugates and DNA sheets loaded with thrombin were assayed at concentrations equivalent to 90 nM thrombin and at a substrate concentration of 2 mg/ml. Reactions were carried out in a volume of 100 μl. The increase in absorbance at 405 nm was monitored on a Beckman DU-30 spectrophotometer.

Thrombin-Loaded Tubular-DNA Nanorobot.

Thrombin-DNA conjugates were mixed with the rectangular DNA origami structures (containing thrombin capture strands) at a molar ratio of 10:1 in 1×TAE-Mg buffer. The mixture was heated to 45° C., then cooled to 25° C. at a rate of 10 min/° C. to facilitate annealing. The thrombin-rectangle-origami assemblies were purified using 100 kD centrifugal filters to remove excess thrombin-DNA conjugates. Tube origami structures were then constructed by adding a 20-fold molar excess of fasteners to the thrombin-rectangle-origami, and then a 5-fold molar excess of additional targeting strands were added to the mixture. To facilitate assembly, the mixture was heated to 37° C., then cooled to 15° C. at a rate of 10 min/° C.

Platelet Aggregation.

Fresh blood from healthy volunteers (informed consent was obtained from all subjects) was collected using ACD (2.5% trisodium citrate, 2.0% D-glucose, 1.5% citric acid) as the anticoagulant. Platelets were washed with CGS buffer (0.123 M NaCl, 0.033 M D-glucose and 0.013 M trisodium citrate, pH 6.5), and resuspended in modified Tyrode's buffer (2.5 mM Hepes, 150 mM NaCl, 2.5 mM KCl, 12 mM NaHCO₃, 5.5 mM D-glucose, pH 7.4), supplemented with 1 mM CaCl₂ and 1 mM MgCl₂, to a final concentration of 3×10⁸/ml. After incubation at 22° C. for 2 h, platelet aggregation was assessed using a turbidometric platelet aggregometer (Xinpusen, Chengdu, China) by adding 0.3 ml of washed platelets and free thrombin, DNA origami sheets, DNA-origami sheets with conjugated thrombin or DNA-origami tubes with conjugated thrombin (at equivalent thrombin concentrations where appropriate) into glass aggregometer cuvettes at 37° C. under stirring.

DNA Nanorobot Preparation for In Vitro and In Vivo Imaging.

Thrombin-loaded rectangular DNA nanostructures with imaging strands were mixed with fluorescent dye-conjugated DNA in 1×TAE-Mg buffer. The molar ratio of dye-modified DNA to each imaging strand was 3:1. The mixtures were heated to 45° C., then cooled to 25° C. at a steady rate of 10 min/° C. using an Eppendorf Mastercycler. Excess dye-modified DNA was removed using 100 kD centrifugal filters. A 20-fold molar excess of fasteners and a 5-fold molar excess of targeting strands, including the AS1411 sequence, were subsequently added. The mixture was heated to 37° C., then cooled to 15° C. at a rate of 10 min/° C. to promote assembly.

Cell Culture.

All cell lines were purchased from the American Type Culture Collection (Manassas, Va., USA) unless stated otherwise. The human breast cancer cell line MDA-MB-231, and human umbilical vein endothelial cells (HUVECs) were maintained in DMEM supplemented with 10% FBS, 100 U/ml penicillin and 100 U/ml streptomycin. The human ovarian cancer cell line SK-OV3 (Shanghai Institutes for Biological Sciences, Shanghai, China) was maintained in McCoy's 5A medium supplemented with 10% FBS, 100 U/ml penicillin and 100 U/ml streptomycin. The murine melanoma cell line B16-F10 was grown in RPMI 1640 medium supplemented with 10% FBS, 1% penicillin and streptomycin. Mouse brain endothelial cells (bEnd.3) were maintained in high glucose DMEM supplemented with 10% FBS, 100 U/ml penicillin and 100 U/ml streptomycin. Cell line authentication was performed by short tandem repeat DNA profiling and comparison with a reference database. The cells were cultured at 37° C., 5% CO₂ and were routinely tested for mycoplasma contamination.

Cell Surface Expression of Nucleolin.

The expression of nucleolin on the cell surface was assessed with antibodies specific to human nucleolin using a Beckman Coulter CyAn ADP flow cytometer (Fullerton, Calif., USA). HUVECs were trypsinized, washed twice in PBS, resuspended in 10% goat serum in PBS, and incubated at 4° C. for 30 min. Cells were then pelleted and resuspended in 2% goat serum in PBS containing 20 μg/ml anti-nucleolin antibody. After one hour incubation on ice, cells were washed three times with PBS and incubated with a 1:500 dilution of FITC-conjugated goat anti-rabbit IgG in PBS for 30 min on ice. Cells were washed with PBS twice and analyzed by flow cytometry.

Cell Surface Binding of AS1411 Aptamer-Containing DNA Strands.

HUVECs were prepared and blocked with goat serum. FITC-labeled F50-containing the AS1411 aptamer sequence (5′-FITC-GGTGGTGGTGGTTGTGGTGGTGGTGG TCTAAAGTTTTGTCGTGAATTGCG-3′, the region of AS1411 aptamer is in bold font) that can recognize nucleolin on the surface of HUVECs was used. Cells were incubated with 2 μM FITC-labeled F50 strands (Invitrogen, Carlsbad, Calif.) or 2 μM random DNA sequence (5′-GAGAACCTGAGTCAGTATTGCGGAGATCTAAAGTTTTGTCGTGAATTGCG-3′) as a control for 2 h at 37° C. Cells were washed twice with PBS and assayed by flow cytometry using a BD Biosciences BD Accuri C6 flow cytometer (San Jose, Calif., USA).

Aptamer Competition Assays.

To determine whether cell surface binding of the F50 DNA strand was specific for nucleolin, HUVECs were pretreated with antibodies against human nucleolin at 45 μg/ml before addition of the aptamer. Cells were then incubated with 2 μM FITC-labeled F50 for 2 h at 37° C. Next, the cells were washed with PBS twice as described above and analyzed by flow cytometry.

Binding of F50 and Fastener Strands.

HUVECs were prepared and treated with goat serum. Cells were then incubated with 2 μM FITC-labeled F50, which are Y-shaped DNA structures with either a 15 base pair duplex (F50+C15) or a 26 base pair duplex (F50+C26) that was annealed in 1×TAE/Mg buffer, for different durations. Cells were washed with PBS twice as detailed above and analyzed by flow cytometry.

The DNA sequences used are shown below:

F50+C15, mixture of fastener:

5′-FITC-
GGTGGTGGTGGTTGTGGTGGTGGTGGTCTAAAGTTTTGTCGTGAATTGC
G-3′
and
5′-GTAAAGCTTTTTTTTTTTTACAACCACCACCACC-3′;

F50+C26, mixture of fully complementary AS1411 portion structures:

5′ end FITC-labeled F50
and
5′-GTAAAGCACCACCACCACCACAACCACCACCACC-3′

Fastener Duplex Opening by Recombinant Nucleolin.

20 μM mixtures of the Y-shaped structures in 1×TAE/Mg buffer were heated to 95° C. and then annealed by cooling to 25° C. at a rate of 3 min/° C. using an Eppendorf thermal cycler. The DNA sequences used are shown below:

F50+C15, mixture of fastener:

5′-FITC-
GGTGGTGGTGGTTGTGGTGGTGGTGGTCTAAAGTTTTGTCGTGAATTGC
G-3′
and
5′-GTAAAGCTTTTTTTTTTTTACAACCACCACCACC-BHQ1-3′;

F50+C26, mixture of fully complementary AS1411 portion structures:

5′-FITC-
GGTGGTGGTGGTTGTGGTGGTGGTGGTCTAAAGTTTTGTCGTGAATTGC
G-3′
and
5′-GTAAAGCACCACCACCACCACAACCACCACCACC-BHQ1-3′

A 10-fold molar excess of recombinant nucleolin (RPC242Hu01, Cloud-Clone Corp.) was added and incubated with the Y-shaped DNA structures (F50+C15 and F50+C26, 2 μM) at 37° C. Fluorescence intensity measurements were performed with a fluorescence spectrometer (LS55, Perkin Elmer).

Fastener Duplex Opening by Cell Surface-Expressed Nucleolin.

The 20 μM mixtures of Y-shaped structures in 1×TAE/Mg buffer were heated to 95° C. and then annealed by cooling to 25° C. at a rate of 3 min/° C. using an Eppendorf thermal cycler.

The DNA sequences used are shown below:

F50+C15, mixture of fastener:

5′-FITC-
GGTGGTGGTGGTTGTGGTGGTGGTGGTCTAAAGTTTTGTCGTGAATTGC
G-3′
and
5′-GTAAAGCTTTTTTTTTTTTACAACCACCACCACC-BHQ1-3′;

F50+C26, mixture of fully complementary AS1411 portion structures:

5′-FITC-
GGTGGTGGTGGTTGTGGTGGTGGTGGTCTAAAGTTTTGTCGTGAATTGC
G-3′
and
5′-GTAAAGCACCACCACCACCACAACCACCACCACC-BHQ1-3′

FC+CC, mixture of no AS1411 structures:

5′-FITC-
AAAAAAAAAAAAAAAAAAAAAAAAAAACTAAAGTTTTGTCGTGAATTGC
G-3′
and
5′-GTAAAGCTTTTTTTTTTTTTTTTTTTTTTTTTTT-BHQ1-3′

HUVECs were prepared and blocked with goat serum as detailed above. Cells were then incubated with the Y-shaped DNA structures (F50+C15, F50+C26 and FC+CC) at a concentration of 2 μM for 2 h at 37° C. Cells were washed twice with PBS and assayed using flow cytometry.

DNA Nanorobot Opening by Recombinant Nucleolin.

Six pairs of fasteners were designed containing 5′-FITC-labeled F50 and 3′-BHQ1-labeled Comp15: FITC-F50-48, Comp15-48-Q; FITC-F50-73, Comp15-73-Q; FITC-F50-97, Comp15-97-Q; FITC-F50-120, Comp15-120-Q; FITC-F50-144, Comp15-144-Q; and FITC-F50-169, Comp15-169-Q. The fluorophore-quencher pairs served as switchable fluorescent beacons. 20 μM mixtures of fluorophore-quencher pairs in 1×TAE/Mg buffer were heated to 95° C. and then annealed by cooling to 25° C. at a rate of 3 min/° C. using an Eppendorf thermal cycler. A 20-fold molar excess of fluorophore-quencher fasteners was added to the DNA sheets. The mixture was heated to 37° C., then cooled to 15° C. at a rate of 10 min/° C. to promote assembly. Extra fastened pairs were removed using filtration devices.

The DNA sequences used are shown below:

FITC-F50-48:
5′-FITC-
GGTGGTGGTGGTTGTGGTGGTGGTGGTCTAAAGTTTTGTCGTGAATTGC
G-3′
Comp15-48-Q:
5′-GTAAAGCTTTTTTTTTTTTACAACCACCACCACC-BHQ1-3′
FITC-F50-73
5′-FITC-
GGTGGTGGTGGTTGTGGTGGTGGTGGTAGAGCTTGACGGGGAAATCAAA
A-3′
Comp15-73-Q
5′-TGTAGCATTTTTTTTTTTTACAACCACCACCACC-BHQ1-3′
FITC-F50-97
5′-FITC-
GGTGGTGGTGGTTGTGGTGGTGGTGGCGAGAAAGGAAGGGAACAAACTA
T-3′
Comp15-97-Q
5′-TGAGTTTCTTTTTTTTTTTACAACCACCACCACC-BHQ1-3′
FITC-F50-120
5′-FITC-
GGTGGTGGTGGTTGTGGTGGTGGTGGATAGGAACCCATGTACAAACAGT
T-3′
Comp15-120-Q
5′-CAAGCCCATTTTTTTTTTTTACAACCACCACCACC-BHQ1-3′
FITC-F50-144
5′-FITC-
GGTGGTGGTGGTTGTGGTGGTGGTGGCACCACCCTCATTTTCCTATTAT
T-3′
Comp15-144-Q
5′-CCGCCAGCTTTTTTTTTTTACAACCACCACCACC-BHQ1′-3′
FITC-F50-169
5′FITC-
GGTGGTGGTGGTTGTGGTGGTGGTGGCTACATTTTGACGCTCACCTGAA
A-3′
Comp15-169-Q
5′-CCCTCAGTTTTTTTTTTTTACAACCACCACCACC-BHQ1-3′

A 20-fold molar excess of recombinant nucleolin was added and incubated with DNA nanorobots labeled by fluorophore-quencher fasteners at 37° C. Fluorescence intensity measurements were performed with a fluorescence spectrometer (LS55, Perkin Elmer).

Cell Surface Nucleolin Triggering of DNA Nanorobot Reconfiguration.

20 μM mixtures of FRET pairs in 1×TAE/Mg buffer were heated to 95° C. and then annealed by cooling to 25° C. at a rate of 3 min/° C. using an Eppendorf thermal cycler. A 20-fold molar excess of FRET fasteners was added to the DNA sheets. The mixture was heated to 37° C., then cooled to 15° C. at a rate of 10 min/° C. to promote assembly.

HUVECs were prepared and blocked with goat serum as described above. Serum-starved HUVECs where surface nucleolin expression was downregulated were also used. Non-starved and serum-starved cells were incubated with DNA nanorobots labeled with fluorophore-quencher fasteners for 2 h at 37° C. Cells were washed twice with PBS and assayed using flow cytometry.

Cell Binding of Nanorobot.

HUVECs were trypsinized, washed twice with 1 ml PBS, seeded onto Lab-Tek Chamber Slides (Nunc) and cultured overnight. Cells were then incubated with either 15 μM Alexa 594-labeled AS1411 aptamer or Alexa 594-labeled nanorobots or nanotubes at a concentration equivalent to 15 μM F50 at 37° C. for different time periods. To determine whether binding of the nanorobot was dependent on surface nucleolin, the cells were pretreated with anti-nucleolin antibodies (45 μg/ml). After treatment, the cells were washed three times with PBS and fixed with 4% paraformaldehyde for 30 min. Then the cells were stained with DIO (plasma membrane) and DAPI (nucleus) (Sigma-Aldrich, St. Louis, Mo.) and imaged with a Nikon Ti-e microscope equipped with an UltraVIEW Vox confocal attachment (Perkin Elmer, Boston, Mass., USA).

Animal Studies.

All animal studies were performed in accordance with ARRIVE guidelines, with the approval of the Ethics of Animal Experiments of the Health Science Center of Peking University Committee. Six to eight-week-old female nude mice (nu/nu) and C57BL/6J mice were obtained from Vital River Laboratory Animal Technology Co. Ltd (Beijing, China) and housed with a 12 h light/dark cycle, at 22° C. and food and water ad libitum. Bama miniature pigs (Sus scrofa domestica) weighing 20 to 25 kg (8-10 months old, half male and half female) were also used in the study. All pigs were healthy and maintained in the animal facility at the Farm Animal Research Center (Beijing, China) under standard conditions prescribed by the Institutional Guidelines. The study protocol was approved by the Institutional Animal Care and Use Committee of the Institute of Zoology, Chinese Academy of Sciences.

In Vivo Targeting.

MDA-MB-231 cells (2.0×10⁶) mixed with an equal volume of Matrigel (BD Pharmingen, San Diego, Calif.), were injected subcutaneously into the mammary fat pads of female nude mice. When the tumor size reached ˜100 mm³, as determined using digital calipers, the mice received tail-vein injections of Cy5.5-labeled DNA nanorobot and were examined using an in vivo optical imaging system (Maestro, CRi Inc., Woburn, Mass., USA) at various time points thereafter (n=3). For biodistribution analysis, the mice were sacrificed at the indicated time points post-injection and the tumors and major organs were harvested (n=3).

Staining of Tissues.

For histological examination, tumors and major organs were collected from MDA-MB-231-bearing nude mice at the indicated time points post-administration of DNA nanorobot-Th and various controls. Tissue samples were fixed in 4% paraformaldehyde, immunostained with anti-CD41 antibody for thrombosis or anti-CD34 antibody for endothelial cells. For evaluating tissue necrosis, sections were stained with hematoxylin and eosin (H&E).

In Vivo Therapeutic Efficacy.

To assess the in vivo efficacy of the nanorobots, nude mice bearing ˜100 mm³ MDA-MB-231 tumors were randomly divided into six groups of eight mice per treatment group and treated with saline, free thrombin, empty nanorobot, nontargetd nanotube-Th, targeted nanotube-Th or nanorobot-Th (˜1.5 U accumulated thrombin/mouse), by tail vein injection every 3 d for a total of 6 treatments. The day of the first injection was designated day 0. Tumors were measured with calipers in three dimensions. The following formula was used to calculate tumor volume: Volume=length×width²/2.

To confirm antitumor efficacy, a syngeneic B16-F10 melanoma tumor model was established by subcutaneous injection of 5×10⁶ murine B16-F10 cells into the right posterior flank of C57BL/6J mice. When the tumors reached a size of ˜150 mm³, the mice (eight mice per group) were treated intravenously with saline, free thrombin, empty nanorobot, nontargeted nanotube-Th, targeted nanotube-Th or nanorobot-Th every other day for 14 d. Tumor volume was determined as described above. The animals were euthanized 2 d after the last treatment, and the livers were excised and weighed. Liver sections were stained with H&E for metastasis analysis.

Two other tumor models, an ovarian cancer SK-OV3 xenograft model and an inducible KRAS^{G12D} lung tumor model were used to investigate the versatility of nanorobot-Th. For the SK-OV3 model, nude mice bearing ˜100 mm³ SK-OV3 xenografts (eight mice per group) were treated intravenously with saline, free thrombin, empty nanorobot, a scrambled aptamer control, nontargeted nanotube-Th, targeted nanotube-Th or nanorobot-Th every 3 d for a total of 6 treatments (˜1.5 U accumulated thrombin/mouse).

The inducible KRAS^{G12D} model was established using transgenic TetO-KRAS^{G12D} mice as described method²⁹. Mice were fed with doxycycline diet since the 6th week after birth to induce primary lung adenomas. After being induced for two weeks, mice with tumors were randomly divided into four groups (three animals per group) and treated with saline, free thrombin, nontargeted nanotube-Th or nanorobot-Th, respectively, by intravenous injection once every 3 d. The progress of lung tumors was monitored by MRI imaging one week and two weeks after treatment started.

In Vivo MRI Imaging.

TetO-KRAS^{G12D} transgenic mice were imaged using a 7.0 T Bruker Biospec animal MRI instrument (Germany). The imaging parameters were set as follows: FOV (field of view)=30×30 cm², MTX (matrix size)=256×256, slice thickness=1 mm, TE=61.2 ms, TR=2320 ms, and NEX=4. The mice were anesthetized with 1.5% isoflurane delivered via nose cone before and during the imaging sessions.

Cell Viability Assay.

The cytotoxicity of DNA nanorobot was assessed in murine endothelial bEnd3 cells. Cells (2000 cells/well) were added to the wells of a 96-well plate (Corning, Woburn, Mass., USA). After culturing at 37° C. for 4 h, the cells were incubated with DNA nanorobot-Th at either 3.3 nM or 6.6 nM (in PBS) for a further 24, 48 or 72 h. The proportion of viable cells was evaluated using a CCK-8 kit (Sigma-Aldrich, St. Louis, Mo., catalog No. 96992). Blank wells only with culture media and PBS-treated wells were used to define 0 and 100% viability, respectively.

Determination of Platelet Surface P-Selectin, Plasma Fibrin and Thrombin Levels and Platelet Counts.

Nude mice bearing MDA-MB-231 tumors were injected intravenously with DNA nanorobot-Th every two days for a total of six injections (n=6 mice). Mouse whole blood was then collected retro-orbitally into a 3.8% sodium citrate solution in blood collection tubes at the indicated time points. For platelet activity studies, the blood was mixed with an equal volume of 2% paraformaldehyde for 30 min at RT and centrifuged to obtain platelet-rich plasma (PRP). The PRP was incubated with FITC-conjugated P-selectin-specific monoclonal antibodies and analyzed by flow cytometry. Fibrin or thrombin levels in the PRP were quantified by enzyme-linked immunosorbent assay (ELISA) with an antibody specific for mouse fibrin (Abeam, ab157527) or thrombin (Abeam ab108844). Platelet numbers were counted manually with a hemocytometer using optical microscopy.

ELISA for Serum Cytokines.

Non-tumor-bearing C57BL/6J mice were injected intravenously with DNA nanorobot-Th every two days for a total of six injections (n=3 mice). Mouse serum was obtained by centrifuging whole blood taken by retro-orbital venous puncture at different time points. Serum cytokine concentrations including IL-6, IP-10, TNFα (R&D Systems, China, Shanghai, China, SM6000B, SMCX100, SMTA00B) and IFNα (ThermoFisher Scientific, Shanghai, China, KMC4011) were measured by ELISA as per the manufacturer's protocol using 50 μl serum³⁸⁻⁴¹.

In Vivo Thrombotic Risk Assessment in Bama Miniature Pigs.

To evaluate the in vivo safety of DNA nanorobot-Th, Bama minipigs were randomly divided into three groups of three pigs per treatment group as follows: group 1, saline; group 2, free thrombin at a dose of 150 U thrombin/pig (equivalent to ˜1.5 U accumulated thrombin/mouse); group 3, nanorobot-Th. The animals were injected via marginal ear vein every other day for a total of three injections. Day 0 marks the first day of injection. Blood was collected from the marginal ear vein into sodium citrate (3.8% final concentration) at different time points (0 h, 2 h, 24 h, 3 d and 5 d) and immediately sent to the Clinical Laboratory, Changping hospital, Beijing, China, for measurements of coagulation parameters. At this institute, normal values for these parameters are as followings: PT: 11.5-15 sec; APTT: 28-43 sec; TT: 13-21 sec; Fibrinogen: 2-4 g/1; D-dimer: 0-0.5 μg/ml. The animals were killed 3 d after the last treatment and the major organs were excised and stained with H&E for histological examination. The following formula was used to perform dose conversion between mice and pigs:

Dp=Dm×(Kmm/Kmp)

where Dp is the dose injected into pigs, Dm is the dose used in mice, Kmm is the Dose in mg/kg to Dose in mg/m² conversion factor of mice and Kmp is the Dose in mg/kg to Dose in mg/m² conversion factor of the pigs.

Blinding:

All experimental procedures and quantification of results, including injections, isolation of the tumors or organs, tissue histological analysis and FACS, were done by two independent researchers.

Statistical Analysis.

Quantitative data are presented as mean±s.d. Tumor volumes were compared using a Kruskal-Wallis test followed by a Mann-Whitney test. The differences in mean volumes between treatment groups were compared using one-way analysis of variance (ANOVA) with repeated measures followed by Tukey's HSD test. Cumulative survival curves in various groups were compared using Kaplan-Meier curves followed by the Log rank test. Two-sided P values less than 0.05 were considered statistically significant. Statistics were performed using SPSS 18.0 software.

### EXAMPLE 1 REFERENCES

1. Seeman, N. C. DNA in a material world. Nature 421, 427-431 (2003).
2. Modi, S., Nizak, C., Surana, S., Halder, S. & Krishnan, Y. Two DNA nanomachines map pH changes along intersecting endocytic pathways inside the same cell. Nat Nanotechnol 8, 459-467 (2013).
3. Jungmann, R. et al. Multiplexed 3D cellular super-resolution imaging with DNA-PAINT and Exchange-PAINT. Nat. Meth. 11, 313-U292 (2014).
4. Bhatia, D., Surana, S., Chakraborty, S., Koushika, S. P. & Krishnan, Y. A synthetic icosahedral DNA-based host-cargo complex for functional in vivo imaging. Nat Commun 2 (2011).
5. Douglas, S. M., Bachelet, I. & Church, G. M. A Logic-Gated Nanorobot for Targeted Transport of Molecular Payloads. Science 335, 831-834 (2012).
6. Lee, H. et al. Molecularly self-assembled nucleic acid nanoparticles for targeted in vivo siRNA delivery. Nat Nanotechnol 7, 389-393 (2012).
Amir, Y. et al. Universal compting by DNA origami robots in a living animal. Nat. Nanotechnol. 9, 353-357 (2014).
8. Chauhan, V. P. & Jain, R. K. Strategies for advancing cancer nanomedicine. Nat. Mater. 12, 958-962 (2013).
9. Huang, X. M. et al. Tumor infarction in mice by antibody-directed targeting of tissue factor to tumor vasculature. Science 275, 547-550 (1997).
10. Hu, P. S. et al. Comparison of three different targeted tissue factor fusion proteins for inducing tumor vessel thrombosis. Cancer Res 63, 5046-5053 (2003).
11. Jain, R. K. Normalization of tumor vasculature: An emerging concept in antiangiogenic therapy. Science 307, 58-62 (2005).
12. Agemy, L. et al. Nanoparticle-induced vascular blockade in human prostate cancer. Blood 116, 2847-2856 (2010).
13. Sambrano, G. R., Weiss, E. J., Zheng, Y. W., Huang, W. & Coughlin, S. R. Role of thrombin signalling in platelets in haemostasis and thrombosis. Nature 413, 74-78 (2001).
14. Pinheiro, A. V., Han, D. R., Shih, W. M. & Yan, H. Challenges and opportunities for structural DNA nanotechnology. Nat Nanotechnol 6, 763-772 (2011).
15. Rothemund, P. W. K. Folding DNA to create nanoscale shapes and patterns. Nature 440, 297-302 (2006).
16. Andersen, E. S. et al. Self-assembly of a nanoscale DNA box with a controllable lid. Nature 459, 73-U75 (2009).
17. Yin, P. et al. Programming DNA tube circumferences. Science 321, 824-826 (2008).
18. Gerling, T., Wagenbauer, K. F., Neuner, A. M. & Dietz, H. Dynamic DNA devices and assemblies formed by shape-complementary, non-base pairing 3D components. Science 347, 1446-1452 (2015).
19. Benson, E. et al. DNA rendering of polyhedral meshes at the nanoscale. Nature 523, 441-U139 (2015).
20. Schuller, V. J. et al. Cellular Immunostimulation by CpG-Sequence-Coated DNA Origami Structures. Acs Nano 5, 9696-9702 (2011).
21. Jiang, Q. et al. DNA Origami as a Carrier for Circumvention of Drug Resistance. J Am Chem Soc 134, 13396-13403 (2012).
22. Zhang, Q. et al. DNA Origami as an In Vivo Drug Delivery Vehicle for Cancer Therapy. Acs Nano 8, 6633-6643 (2014).
23. Chen, Y. J., Groves, B., Muscat, R. A. & Seelig, G. DNA nanotechnology from the test tube to the cell. Nat Nanotechnol 10, 748-760 (2015).
24. Soundararajan, S., Chen, W. W., Spicer, E. K., Courtenay-Luck, N. & Fernandes, D. J. The nucleolin targeting aptamer AS1411 destabilizes bcl-2 messenger RNA in human breast cancer cells. Cancer Res 68, 2358-2365 (2008).
25. Huang, Y. et al. The angiogenic function of nucleolin is mediated by vascular endothelial growth factor and nonmuscle myosin. Blood 107, 3564-3571 (2006).
26. Nutiu, R. & Li, Y. F. Structure-switching signaling aptamers. J Am Chem Soc 125, 4771-4778 (2003).
27. Hoshyar et al. The effect of nanoparticle size on in vivo pharmacokinetics and cellular interaction. Nanomedicine 11, 673-692 (2016).
28. Kong et al., Hyperthermia Enables Tumor-specific Nanoparticle Delivery: Effect of Particle Size, Cancer Research 60, 4440-4445 (2000).
29. Zhang H, et al. Identification of urine protein biomarkers with the potential for early detection of lung cancer. Sci Rep 5, 11805 (2015)
30. Liu, X. W. et al. A DNA Nanostructure Platform for Directed Assembly of Synthetic Vaccines. Nano Lett 12, 4254-4259 (2012).
31. Surana, S., Shenoy, A. R. & Krishnan, Y. Designing DNA nanodevices for compatibility with the immune system of higher organisms. Nat Nanotechnol 10, 741-747 (2015).
32. Liu, Y., Zeng, B. H., Shang, H. T., Cen, Y. Y. & Wei, H. Bama Miniature Pigs (Sus scrofa domestica) as a Model for Drug Evaluation for Humans: Comparison of In Vitro Metabolism and In Vivo Pharmacokinetics of Lovastatin. Comparative Med 58, 580-587 (2008).
33. Brown, D. B. et al. Quality Improvement Guidelines for Transhepatic Arterial Chemoembolization, Embolization, and Chemotherapeutic Infusion for Hepatic Malignancy. J Vasc Interv Radiol 23, 287-294 (2012).
34. Goldberg, S. N. et al. Image-guided tumor ablation: Proposal for standardization of terms and reporting criteria. Radiology 228, 335-345 (2003).
35. Miniard A C, et al. Nucleolin binds to a subset of selenoprotein mRNAs and regulates their expression. Nucl. Acids Res. 38, 4807-4820 (2010).
36. Thompson J S, et al. BAFF binds to the tumor necrosis factor receptor-like molecule B cell maturation antigen and is important for maintaining the peripheral B cell population. J Exp Med 192, 129-136 (2000).
37. Douglas, S. M., Chou, J. J. & Shih, W. M. DNA-nanotube-induced alignment of membrane proteins for NMR structure determination. Proc. Natl. Acad. Sci. U.S.A. 104, 6644-6648 (2007).
38. Alvarez-Erviti L, et al. Delivery of siRNA to the mouse brain by systemic injection of targeted exosomes. Nat Biotechnol 29, 341-345 (2011).
39. Guyer R A, Macara I G. Loss of the polarity protein PAR3 activates STAT3 signaling via an atypical protein kinase C (aPKC)/NF-κB/interleukin-6 (IL-6) axis in mouse mammary cells. J Biol Chem 290, 8457-8468 (2015).
40. Gottfries J, Melgar S, Michaëlsson E. Modelling of mouse experimental colitis by global property screens: a holistic approach to assess drug effects in inflammatory bowel disease. PloS one, 7, e30005 (2012).
41. O'Callaghan P, et al. Microglial heparan sulfate proteoglycans facilitate the cluster-of-differentiation 14 (CD14)/Toll-like receptor 4 (TLR4)-dependent inflammatory response. J Biol Chem 290, 14904-14914 (2015).

Supplementary Materials

DNA Origami Design

DNA origami is constructed in a honeycomb lattice or a square lattice, in which the rule of DNA helicity enables customized orientation of the free ends of staple strands [1]. In the present study, the rectangular DNA origami sheet is assembled into a square lattice, which means the backbone of the DNA strand rotates 270° at 8 bp intervals. This enables the free ends of the staple strands, 32 nt in length, to all lay on the same side of the rectangular DNA sheet surface. As shown in FIG. 19, each gray strand represents a 32 nt staple strand whose end can be extended with sequence specific overhangs as functional linkers, enabling the addressability of the DNA origami. For the purpose of this study define the “top” surface is defined as that side which contains the functional linkers, referring to the other surface as the “bottom”. Importantly, the top and bottom surfaces are distinct from each other in terms of curvature [1]. Thus, thrombin molecules can be loaded on the top surface, following the assembly code in FIG. 20, or loaded on the bottom surface by modifying the orientation of the functional linkers to form the control structure (ctrl-rect-DNA-origami, FIG. 21).

Characterization of Free Thrombin, Thrombin-DNA Conjugates and Thrombin-Bounded DNA Sheet.

Cy5-modified oligonucleotides were conjugated to thrombin molecules via the 5′-thiol using the sulfo-SMCC chemistry described above. It was estimated that polyT DNA-labeled thrombin had an average DNA-to-protein ratio of 2.5±0.8; we use this average value of 2.5 for further calculations. Quantification of Cy5-DNA-labeled enzyme-bound origami sheet was carried out by UV-Vis spectrometry. The DNA origami concentration was quantified by measuring the absorbance at 260 nm (A260) using an extinction coefficient of 1.09×108M-1cm-1. The Cy5-S15-Thrombin was quantified by measuring the absorbance at 650 nm (A650) using an extinction coefficient of 2.5×105 M-1cm-1 [2]. The average number of thrombins on the DNA origami was calculated as follows:

Ratio(thrombin/origami)=CCy5-S15-thrombin/Corigami=(CCy5-S15/2.5)/Corigami=3.8±0.4

Additional Characterization of DNA Robots

Additional characterization of DNA robots is shown in FIGS. 29A-29F.

Thrombin molecules can be loaded on the top or the bottom surface of origami sheets. After fastening of the rectangular sheet into a tube, the top surface will be preferentially rolled inside of the tube due to curvature driving forces. Thus, thrombin can be loaded on the inside or the outside surface of origami tubes. Only thrombin loaded inside tubes can be protected and shielded before delivery to the target location in vivo. We estimated the topography through a platelet aggregation assay. For the rectangular DNA sheet structures, the long single stranded scaffold strand 7249 nt M13 phage DNA and short strands, including basic staple strands (gray strands in FIG. 20) and thrombin-loading strands (yellow strands in FIG. 20) were mixed in 1×TAE-Mg buffer. The mixtures were then placed on an Eppendorf thermal cycler with the program: rapid heating to 65° C., then cooling to 25° C. at a rate of 10 min/° C. for annealing. For the control rectangular sheet structures, M13 and short strands, including basic staple and thrombin-loading strands (purple strands in FIGS. 21), were mixed together and annealed. Thrombin-DNA conjugates were then mixed with the rectangular DNA sheet or control nanostructure. The mixtures were heated to 45° C. and cooled to 25° C. at a rate of 10 min/° C. to facilitate annealing. The resultant thrombin-rectangle-origami assemblies (rect-DNA-origami-Th and ctrl-rect-DNA-origami-Th) were purified using 100 kD centrifugal filters to remove excess thrombin-DNA conjugates. After loading thrombin, a 20-fold molar excess of fasteners and a 5-fold molar excess of targeting strands, were added to induce the formation of tube structures (nanorobot-Th and ctrl-tube-DNA origami-Th). To facilitate annealing, the mixture was heated to 37° C. and then cooled to 15° C. at a rate of 10 min/° C.

The thrombin-loaded rectangular and tubular DNA origami nanostructures were applied to the platelet aggregation assay mentioned above. Additionally, nanorobot-Th and ctrl-tube-DNA origami-Th were pretreated with proteinase K for 15 min at room temperature to remove the thrombin molecules on the outside surface of the tubes. Next, the proteinase-treated nanostructures were degraded with 20 U/ml DNase I (Invitrogen, Carlsbad, Calif., USA) at 37° C. for 30 min to expose the thrombin molecules inside the tubes. The considerably low platelet aggregation using nanorobot-Thor ctrl-tube-DNA origami-Th structures without such treatment indicates that only a small amount of thrombin molecules loaded onto the outside surface of the tubes. On the other hand, aggregation results of tube structures after proteinase and DNase I treatment elicited potent platelet aggregation, reflecting the vast majority of thrombin loaded inside the tubes. The results of these experiments were used to estimate the percentage of thrombin loaded on the inside and outside surfaces of nanorobot-Th and ctrl-tube-DNA origami-Th. The data in FIGS. 24A-24B and 25 indicate that approximately 84% of the thrombin was loaded inside the nanorobot-Thin which the thrombin-DNA-binding sites were designed to be on the “top” surface. About 16% of the thrombin was loaded inside the ctrl-tube origami where the binding sites were designed on the “bottom” surface. These results demonstrate that the thrombin molecules can be arranged inside the DNA nanostructure in a shielded state by our design. Furthermore, this is in agreement with the curvature preference of rectangular DNA origami structures as predicted by a computer simulation of the twist model [3]. Our results also agree well with a study demonstrating that the opposite corners of the rectangular DNA nanostructure adopt a bend in the same direction [4].

Supplementary characterization of the nanorobot functions are shown in FIGS. 31, 32A-E, 33A-C, 34, 35, 36, 37, 38A-38B, and 39A-39D.

Characterization of in vivo nanorobot targeting and activity is shown in FIGS. 40A-C, 41A-C, 42A-D, 43A-E, and 44A-D.

Safety assessment of the thrombin-loaded nanorobot is shown in FIGS. 45A-F, 46, 47, and 48A-F.

TABLE 1
List of functional strands and staple strands pool.

| Functional Strands Name | Strand codes |
|---|---|
| Imaging strands* | I-31, I-32, I-34, I-35, I-42, I-45, I-55, |
| | I-56, I-66, I-67, I-76, I-77, I-79, I-80, |
| | I-87, I-90, I-103, I-104, I-114, I-115, |
| | I-127, I-128, I-130, I-131, I-138, I- |
| | 141, I-151, I-152, I-162, I-163, I-172, |
| | I-173, I-175, I-176, I-183, I-186, I- |
| | 187 |
| Top surface thrombin | TH-43, TH-44, TH-57, TH-64, TH- |
| capture strands | 65, TH-78, TH-139, TH-140, TH-153, |
| | TH-160, TH-161, TH-174 |
| Fastener strands | F50-48, Comp15-48, F50-73, |
| | Comp15-73, F50-97, Comp15-97, |
| | F50-120, Comp15-120, F50-144, |
| | Comp15-144, F50-145, F50-169, |
| | Comp15-169 |
| Additional targeting strands | T-1, T-2, T-11, T-12, T-205, T-206, |
| including AS1411 | T-215, T-216 |
| Bottom surface thrombin | 44-43, 43-42, 56-57, 65-64, 64-63, |
| capture strands | 77-78, 140-139, 139-138, 152-153, |
| | 161-160, 160-159, 173-174 |

All the DNA strands were purchased from the oligonucleotide synthesis service of Invitrogen.
*The Alexa-594/FITC/Cy5.5 labeled DNA for in vitro and in vivo imaging:
5′-TAAACTCTTTGCGCAC-Alexa-594/FITC/Cy5.5-3′ Every capture strand for imaging contains an extended overhang at the 5′-end that is complementary to the Alexa-594/FITC/Cy5.5 labeled DNA.

TABLE 2
Hind-limb paralysis (>15 min) and death of mice after
thrombin^{§} injection.

Severity of thrombus formation

| Thrombin injected | Paralysis ratio % | Death ratio % |
|---|---|---|
| (U/mouse) | (n) | (n) |
| 0 | 0 (5) | 0 (5) |
| 1.15 | 0 (5) | 0 (5) |
| 2.30 | 0 (5) | 0 (5) |
| 3.45 | 0 (8) | 0 (8) |
| 4.60 | 100 (8) | 37.5 (8) |
| 15 | 100 (8) | 100 (8) |

^{§}Thrombin was intravenously injected to estimate the severity of microthrombus formation. The hind-limb paralysis rate or death rate was expressed as the ratio of paralyzed or dead mice to the total animals used in each group. All mice injected with more than 15.0 U of thrombin died of acute thromboembolism. Figures in parentheses indicate total number of mice used.

TABLE 3
Comparison of PT, APTT and TT values^{†} and plasma levels of fibrinogen and
D-dimer between thrombin-treated minipigs^{§} and control groups.

| | | | PT | APTT | TT | Fibrinogen | D-dimer |
|---|---|---|---|---|---|---|---|
| Time | | Group | (sec) | (sec) | (sec) | (g/l) | (μg/ml) |
| 0 | h | Saline | 13.9 (±0.06) | 28.6 (±0.38) | 20.4 (±0.15) | 2.81 (±0.10) | 0.68 (±0.08) |
| | | 150 U | 13.9 (±0.10) | 27.6 (±2.86) | 20.0 (±1.30) | 3.13 (±0.92) | 0.75 (±0.07) |
| | | 350 U | 14.0 (±0.25) | 29.8 (±3.30) | 20.9 (±1.36) | 2.87 (±0.59) | 1.04 (±0.22)* |
| 2 | h | Saline | 14.7 (±0.21) | 27.0 (±0.56) | 20.1 (±0.25) | 2.78 (±0.12) | 0.71 (±0.08) |
| | | 150 U | 14.7 (±0.23) | 29.7 (±2.66) | 20.9 (±1.53) | 3.02 (±0.92) | 0.74 (±0.24) |
| | | 350 U | 14.9 (±0.26) | 39.3 (±6.56)* | 19.9 (±0.26) | 2.81 (±0.60) | 1.07 (±0.28)* |
| 24 | h | Saline | 14.6 (±0.10) | 29.3 (±0.28) | 20.4 (±0.27) | 2.58 (±0.21) | 0.69 (±0.06) |
| | | 150 U | 14.0 (±0.40) | 27.3 (±7.77) | 19.4 (±1.97) | 3.33 (±0.50) | 0.92 (±0.53) |
| | | 350 U | 14.7 (±0.17) | 42.7 (±12.35)* | 19.4 (±0.82) | 3.13 (±0.69) | 1.09 (±0.29)* |
| 3 | d | Saline | 14.7 (±0.15) | 30.3 (±0.38) | 22.7 (±0.42) | 2.74 (±0.14) | 0.74 (±0.02) |
| | | 150 U | 13.8 (±0.50) | 28.9 (±2.38) | 21.0 (±2.25) | 4.05 (±0.46) | 0.70 (±0.10) |
| | | 350 U | 14.1 (±0.29) | 39.3 (±5.02)* | 20.1 (±0.10) | 3.42 (±0.73) | 1.32 (±0.41)* |
| 5 | d | Saline | 14.5 (±0.15) | 28.6 (±0.10) | 21.0 (±0.30) | 2.47 (±0.41) | 0.72 (±0.02) |
| | | 150 U | 14.1 (±0.20) | 36.6 (±5.82) | 20.1 (±1.93) | 3.85 (±0.79) | 0.69 (±0.05) |
| | | 350 U | 14.7 (±0.35) | 41.5 (±4.26)* | 20.4 (±1.05) | 3.10 (±0.54) | 1.12 (±0.20)* |
| 8 | d | Saline | 13.8 (±0.05) | 29.1 (±0.35) | 20.7 (±0.15) | 2.91 (±0.20) | 0.68 (±0.09) |
| | | 150 U | 14.2 (±0.29) | 29.9 (±0.38) | 19.7 (±0.81) | 3.50 (±0.50) | 0.70 (±0.08) |
| | | 350 U | 13.9 (±0.15) | 28.3 (±0.35) | 19.9 (±0.40) | 2.68 (±0.43) | 0.70 (±0.10) |

^{†}PT prothrombin time (institutional normal range: 11.5-15), APTT activated partial thromboplastin time (institutional normal range: 28-43), TT thrombin time (institutional normal range: 13-21), Fibrinogen (institutional normal range: 2-4), D-dimer (institutional normal range: 0-0.5).
^{§}Different doses of free thrombin were injected into pigs via marginal ear vein once or three times. At the indicated time points, blood was drawn from the marginal ear vein and plasma was prepared. Plasma PT, APTT and TT and levels of fibrinogen and D-dimer were measured. Compared to controls, there were no differences in any plasma coagulation parameter after single or multiple administrations of thrombin at 150 U thrombin/pig. All pigs injected with more than 1,540 U/pig (equivalent to ~15.0 U/mouse) died of acute thromboembolism, consistent with the lethal dose obtained from mice. 0 h, before injection.
*p < 0.05 vs. saline groups at the indicated time points. Data are shown as mean ± s.d. of three animals.
*APTT prolongation occurred but <43 s after single or multiple injections of thrombin at 350 U/pig; D-dimer content increased after injection of thrombin of 350 U/pig, but disappeared with a final measurement of normal levels 3 d after the last treatment, indicating a transient and reversible coagulation activation.

SEQUENCES OF STAPLE STRAND POOL AND
FUNCTIONAL STAPLE STRANDS
Staple strands pool (5′-3′):

| | |
|---|---|
| 13 | TGGTTTTTAACGTCAAAGGGCGAAGAACCATC |
| 14 | CTTGCATGCATTAATGAATCGGCCCGCCAGGG |
| 15 | TAGATGGGGGGTAACGCCAGGGTTGTGCCAAG |
| 16 | CATGTCAAGATTCTCCGTGGGAACCGTTGGTG |
| 17 | CTGTAATATTGCCTGAGAGTCTGGAAAACTAG |
| 18 | TGCAACTAAGCAATAAAGCCTCAGTTATGACC |
| 19 | AAACAGTTGATGGCTTAGAGCTTATTTAAATA |
| 20 | ACGAACTAGCGTCCAATACTGCGGAATGCTTT |
| 21 | CTTTGAAAAGAACTGGCTCATTATTTAATAAA |
| 22 | ACGGCTACTTACTTAGCCGGAACGCTGACCAA |
| 23 | GAGAATAGCTTTTGCGGGATCGTCGGGTAGCA |
| 24 | ACGTTAGTAAATGAATTTTCTGTAAGCGGAGT |
| 25 | ACCCAAATCAAGTTTTTTGGGGTCAAAGAACG |
| 26 | TGGACTCCCTTTTCACCAGTGAGACCTGTCGT |
| 27 | GCCAGCTGCCTGCAGGTCGACTCTGCAAGGCG |
| 28 | ATTAAGTTCGCATCGTAACCGTGCGAGTAACA |
| 29 | ACCCGTCGTCATATGTACCCCGGTAAAGGCTA |
| 30 | TCAGGTCACTTTTGCGGGAGAAGCAGAATTAG |
| 31 | CAAAATTAAAGTACGGTGTCTGGAAGAGGTCA |
| 32 | TTTTTGCGCAGAAAACGAGAATGAATGTTTAG |
| 33 | ACTGGATAACGGAACAACATTATTACCTTATG |
| 34 | CGATTTTAGAGGACAGATGAACGGCGCGACCT |
| 35 | GCTCCATGAGAGGCTTTGAGGACTAGGGAGTT |
| 36 | AAAGGCCGAAAGGAACAACTAAAGCTTTCCAG |
| 37 | AGCTGATTACAAGAGTCCACTATTGAGGTGCC |
| 38 | CCCGGGTACTTTCCAGTCGGGAAACGGGCAAC |
| 39 | GTTTGAGGGAAAGGGGGATGTGCTAGAGGATC |
| 40 | AGAAAAGCAACATTAAATGTGAGCATCTGCCA |
| 41 | CAACGCAATTTTTGAGAGATCTACTGATAATC |
| 42 | TCCATATACATACAGGCAAGGCAACTTTATTT |
| 43 | CAAAAATCATTGCTCCTTTTGATAAGTTTCAT |
| 44 | AAAGATTCAGGGGGTAATAGTAAACCATAAAT |
| 45 | CCAGGCGCTTAATCATTGTGAATTACAGGTAG |
| 46 | TTTCATGAAAATTGTGTCGAAATCTGTACAGA |
| 47 | AATAATAAGGTCGCTGAGGCTTGCAAAGACTT |
| 48 | CGTAACGATCTAAAGTTTTGTCGTGAATTGCG |
| 49 | GTAAAGCACTAAATCGGAACCCTAGTTGTTCC |
| 50 | AGTTTGGAGCCCTTCACCGCCTGGTTGCGCTC |
| 51 | ACTGCCCGCCGAGCTCGAATTCGTTATTACGC |
| 52 | CAGCTGGCGGACGACGACAGTATCGTAGCCAG |
| 53 | CTTTCATCCCCAAAAACAGGAAGACCGGAGAG |
| 54 | GGTAGCTAGGATAAAAATTTTTAGTTAACATC |
| 55 | CAATAAATACAGTTGATTCCCAATTTAGAGAG |
| 56 | TACCTTTAAGGTCTTTACCCTGACAAAGAAGT |
| 57 | TTTGCCAGATCAGTTGAGATTTAGTGGTTTAA |
| 58 | TTTCAACTATAGGCTGGCTGACCTTGTATCAT |
| 59 | CGCCTGATGGAAGTTTCCATTAAACATAACCG |
| 60 | ATATATTCTTTTTTCACGTTGAAAATAGTTAG |
| 61 | GAGTTGCACGAGATAGGGTTGAGTAAGGGAGC |
| 62 | TCATAGCTACTCACATTAATTGCGCCCTGAGA |
| 63 | GAAGATCGGTGCGGGCCTCTTCGCAATCATGG |
| 64 | GCAAATATCGCGTCTGGCCTTCCTGGCCTCAG |
| 65 | TATATTTTAGCTGATAAATTAATGTTGTATAA |
| 66 | CGAGTAGAACTAATAGTAGTAGCAAACCCTCA |
| 67 | TCAGAAGCCTCCAACAGGTCAGGATCTGCGAA |
| 68 | CATTCAACGCGAGAGGCTTTTGCATATTATAG |
| 69 | AGTAATCTTAAATTGGGCTTGAGAGAATACCA |
| 70 | ATACGTAAAAGTACAACGGAGATTTCATCAAG |
| 71 | AAAAAAGGACAACCATCGCCCACGCGGGTAAA |
| 72 | TGTAGCATTCCACAGACAGCCCTCATCTCCAA |
| 73 | CCCCGATTTAGAGCTTGACGGGGAAATCAAAA |
| 74 | GAATAGCCGCAAGCGGTCCACGCTCCTAATGA |
| 75 | GTGAGCTAGTTTCCTGTGTGAAATTTGGGAAG |
| 76 | GGCGATCGCACTCCAGCCAGCTTTGCCATCAA |
| 77 | AAATAATTTTAAATTGTAAACGTTGATATTCA |
| 78 | ACCGTTCTAAATGCAATGCCTGAGAGGTGGCA |
| 79 | TCAATTCTTTTAGTTTGACCATTACCAGACCG |
| 80 | GAAGCAAAAAAGCGGATTGCATCAGATAAAAA |
| 81 | CCAAAATATAATGCAGATACATAAACACCAGA |
| 82 | ACGAGTAGTGACAAGAACCGGATATACCAAGC |
| 83 | GCGAAACATGCCACTACGAAGGCATGCGCCGA |
| 84 | CAATGACACTCCAAAAGGAGCCTTACAACGCC |
| 85 | CCAGCAGGGGCAAAATCCCTTATAAAGCCGGC |
| 86 | GCTCACAATGTAAAGCCTGGGGTGGGTTTGCC |
| 87 | GCTTCTGGTCAGGCTGCGCAACTGTGTTATCC |
| 88 | GTTAAAATTTTAACCAATAGGAACCCGGCACC |
| 89 | AGGTAAAGAAATCACCATCAATATAATATTTT |
| 90 | TCGCAAATGGGGCGCGAGCTGAAATAATGTGT |
| 91 | AAGAGGAACGAGCTTCAAAGCGAAGATACATT |
| 92 | GGAATTACTCGTTTACCAGACGACAAAAGATT |
| 93 | CCAAATCACTTGCCCTGACGAGAACGCCAAAA |
| 94 | AAACGAAATGACCCCCAGCGATTATTCATTAC |
| 95 | TCGGTTTAGCTTGATACCGATAGTCCAACCTA |
| 96 | TGAGTTTCGTCACCAGTACAAACTTAATTGTA |
| 97 | GAACGTGGCGAGAAAGGAAGGGAACAAACTAT |
| 98 | CCGAAATCCGAAAATCCTGTTTGAAGCCGGAA |
| 99 | GCATAAAGTTCCACACAACATACGAAGCGCCA |
| 100 | TTCGCCATTGCCGGAAACCAGGCATTAAATCA |
| 101 | GCTCATTTTCGCATTAAATTTTTGAGCTTAGA |
| 102 | AGACAGTCATTCAAAAGGGTGAGAAGCTATAT |
| 103 | TTTCATTTGGTCAATAACCTGTTTATATCGCG |
| 104 | TTTTAATTGCCCGAAAGACTTCAAAACACTAT |
| 105 | CATAACCCGAGGCATAGTAAGAGCTTTTTAAG |
| 106 | GAATAAGGACGTAACAAAGCTGCTCTAAAACA |
| 107 | CTCATCTTGAGGCAAAAGAATACAGTGAATTT |
| 108 | CTTAAACATCAGCTTGCTTTCGAGCGTAACAC |
| 109 | ACGAACCAAAACATCGCCATTAAATGGTGGTT |
| 110 | CGACAACTAAGTATTAGACTTTACAATACCGA |
| 111 | CTTTTACACAGATGAATATACAGTAAACAATT |
| 112 | TTAAGACGTTGAAAACATAGCGATAACAGTAC |
| 113 | GCGTTATAGAAAAAGCCTGTTTAGAAGGCCGG |
| 114 | ATCGGCTGCGAGCATGTAGAAACCTATCATAT |
| 115 | CCTAATTTACGCTAACGAGCGTCTAATCAATA |
| 116 | AAAAGTAATATCTTACCGAAGCCCTTCCAGAG |
| 117 | TTATTCATAGGGAAGGTAAATATTCATTCAGT |
| 118 | GAGCCGCCCCACCACCGGAACCGCGACGGAAA |
| 119 | AATGCCCCGTAACAGTGCCCGTATCTCCCTCA |
| 120 | CAAGCCCAATAGGAACCCATGTACAAACAGTT |
| 121 | CGGCCTTGCTGGTAATATCCAGAACGAACTGA |
| 122 | TAGCCCTACCAGCAGAAGATAAAAACATTTGA |
| 123 | GGATTTAGCGTATTAAATCCTTTGTTTTCAGG |
| 124 | TTTAACGTTCGGGAGAAACAATAATTTTCCCT |
| 125 | TAGAATCCCTGAGAAGAGTCAATAGGAATCAT |
| 126 | AATTACTACAAATTCTTACCAGTAATCCCATC |
| 127 | CTAATTTATCTTTCCTTATCATTCATCCTGAA |
| 128 | TCTTACCAGCCAGTTACAAAATAAATGAAATA |
| 129 | GCAATAGCGCAGATAGCCGAACAATTCAACCG |
| 130 | ATTGAGGGTAAAGGTGAATTATCAATCACCGG |
| 128 | AACCAGAGACCCTCAGAACCGCCAGGGGTCAG |
| 132 | TGCCTTGACTGCCTATTTCGGAACAGGGATAG |
| 133 | AGGCGGTCATTAGTCTTTAATGCGCAATATTA |
| 134 | TTATTAATGCCGTCAATAGATAATCAGAGGTG |
| 135 | CCTGATTGAAAGAAATTGCGTAGACCCGAACG |
| 136 | ATCAAAATCGTCGCTATTAATTAACGGATTCG |
| 137 | ACGCTCAAAATAAGAATAAACACCGTGAATTT |
| 138 | GGTATTAAGAACAAGAAAAATAATTAAAGCCA |
| 139 | ATTATTTAACCCAGCTACAATTTTCAAGAACG |
| 140 | GAAGGAAAATAAGAGCAAGAAACAACAGCCAT |
| 141 | GACTTGAGAGACAAAAGGGCGACAAGTTACCA |
| 142 | GCCACCACTCTTTTCATAATCAAACCGTCACC |
| 143 | CTGAAACAGGTAATAAGTTTTAACCCCTCAGA |
| 144 | CTCAGAGCCACCACCCTCATTTTCCTATTATT |
| 145 | CCGCCAGCCATTGCAACAGGAAAAATATTTTT |
| 146 | GAATGGCTAGTATTAACACCGCCTCAACTAAT |
| 147 | AGATTAGATTTAAAAGTTTGAGTACACGTAAA |
| 148 | ACAGAAATCTTTGAATACCAAGTTCCTTGCTT |
| 149 | CTGTAAATCATAGGTCTGAGAGACGATAAATA |
| 150 | AGGCGTTACAGTAGGGCTTAATTGACAATAGA |
| 151 | TAAGTCCTACCAAGTACCGCACTCTTAGTTGC |
| 152 | TATTTTGCTCCCAATCCAAATAAGTGAGTTAA |
| 153 | GCCCAATACCGAGGAAACGCAATAGGTTTACC |
| 154 | AGCGCCAACCATTTGGGAATTAGATTATTAGC |
| 155 | GTTTGCCACCTCAGAGCCGCCACCGATACAGG |
| 156 | AGTGTACTTGAAAGTATTAAGAGGCCGCCACC |
| 157 | GCCACGCTATACGTGGCACAGACAACGCTCAT |
| 158 | ATTTTGCGTCTTTAGGAGCACTAAGCAACAGT |
| 159 | GCGCAGAGATATCAAAATTATTTGACATTATC |
| 160 | TAACCTCCATATGTGAGTGAATAAACAAAATC |
| 161 | CATATTTAGAAATACCGACCGTGTTACCTTTT |
| 162 | CAAGCAAGACGCGCCTGTTTATCAAGAATCGC |
| 163 | TTTTGTTTAAGCCTTAAATCAAGAATCGAGAA |
| 164 | ATACCCAAGATAACCCACAAGAATAAACGATT |
| 165 | AATCACCAAATAGAAAATTCATATATAACGGA |
| 166 | CACCAGAGTTCGGTCATAGCCCCCGCCAGCAA |
| 167 | CCTCAAGAATACATGGCTTTTGATAGAACCAC |
| 168 | CCCTCAGAACCGCCACCCTCAGAACTGAGACT |
| 169 | GGAAATACCTACATTTTGACGCTCACCTGAAA |
| 170 | GCGTAAGAGAGAGCCAGCAGCAAAAAGGTTAT |
| 171 | CTAAAATAGAACAAAGAAACCACCAGGGTTAG |
| 172 | AACCTACCGCGAATTATTCATTTCCAGTACAT |
| 173 | AAATCAATGGCTTAGGTTGGGTTACTAAATTT |
| 174 | AATGGTTTACAACGCCAACATGTAGTTCAGCT |
| 175 | AATGCAGACCGTTTTTATTTTCATCTTGCGGG |
| 176 | AGGTTTTGAACGTCAAAAATGAAAGCGCTAAT |
| 177 | ATCAGAGAAAGAACTGGCATGATTTTATTTTG |
| 178 | TCACAATCGTAGCACCATTACCATCGTTTTCA |
| 179 | TCGGCATTCCGCCGCCAGCATTGACGTTCCAG |
| 180 | TAAGCGTCGAAGGATTAGGATTAGTACCGCCA |
| 181 | CTAAAGCAAGATAGAACCCTTCTGAATCGTCT |
| 182 | CGGAATTATTGAAAGGAATTGAGGTGAAAAAT |
| 183 | GAGCAAAAACTTCTGAATAATGGAAGAAGGAG |
| 184 | TATGTAAACCTTTTTTAATGGAAAAATTACCT |
| 185 | AGAGGCATAATTTCATCTTCTGACTATAACTA |
| 186 | TCATTACCCGACAATAAACAACATATTTAGGC |
| 187 | CTTTACAGTTAGCGAACCTCCCGACGTAGGAA |
| 188 | TTATTACGGTCAGAGGGTAATTGAATAGCAGC |
| 189 | CCGGAAACACACCACGGAATAAGTAAGACTCC |
| 190 | TGAGGCAGGCGTCAGACTGTAGCGTAGCAAGG |
| 191 | TGCTCAGTCAGTCTCTGAATTTACCAGGAGGT |
| 192 | TATCACCGTACTCAGGAGGTTTAGCGGGGTTT |
| 193 | GAAATGGATTATTTACATTGGCAGACATTCTG |
| 194 | GCCAACAGTCACCTTGCTGAACCTGTTGGCAA |
| 195 | ATCAACAGTCATCATATTCCTGATTGATTGTT |
| 196 | TGGATTATGAAGATGATGAAACAAAATTTCAT |
| 197 | TTGAATTATGCTGATGCAAATCCACAAATATA |
| 198 | TTTTAGTTTTTCGAGCCAGTAATAAATTCTGT |
| 199 | CCAGACGAGCGCCCAATAGCAAGCAAGAACGC |
| 200 | GAGGCGTTAGAGAATAACATAAAAGAACACCC |
| 201 | TGAACAAACAGTATGTTAGCAAACTAAAAGAA |
| 202 | ACGCAAAGGTCACCAATGAAACCAATCAAGTT |
| 203 | TGCCTTTAGTCAGACGATTGGCCTGCCAGAAT |
| 204 | GGAAAGCGACCAGGCGGATAAGTGAATAGGTG |

### For In Vitro and In Vivo Imaging:

I-31
GTGCGCAAAGAGTTTACAAAATTAAAGTACGGTGTCTGGAAGAGGTCA
I-32
GTGCGCAAAGAGTTTATTTTTGCGCAGAAAACGAGAATGAATGTTTAG
I-34
GTGCGCAAAGAGTTTACGATTTTAGAGGACAGATGAACGGCGCGACCT
I-35
GTGCGCAAAGAGTTTAGCTCCATGAGAGGCTTTGAGGACTAGGGAGTT
I-42
GTGCGCAAAGAGTTTATCCATATACATACAGGCAAGGCAACTTTATTT
I-45
GTGCGCAAAGAGTTTACCAGGCGCTTAATCATTGTGAATTACAGGTAG
I-55
GTGCGCAAAGAGTTTACAATAAATACAGTTGATTCCCAATTTAGAGAG
I-56
GTGCGCAAAGAGTTTATACCTTTAAGGTCTTTACCCTGACAAAGAAGT
I-66
GTGCGCAAAGAGTTTACGAGTAGAACTAATAGTAGTAGCAAACCCTCA
I-67
GTGCGCAAAGAGTTTATCAGAAGCCTCCAACAGGTCAGGATCTGCGAA
I-76
GTGCGCAAAGAGTTTAGGCGATCGCACTCCAGCCAGCTTTGCCATCAA
I-77
GTGCGCAAAGAGTTTAAAATAATTTTAAATTGTAAACGTTGATATTCA
I-79
GTGCGCAAAGAGTTTATCAATTCTTTTAGTTTGACCATTACCAGACCG
I-80
GTGCGCAAAGAGTTTAGAAGCAAAAAAGCGGATTGCATCAGATAAAAA
I-87
GTGCGCAAAGAGTTTAGCTTCTGGTCAGGCTGCGCAACTGTGTTATCC
I-90
GTGCGCAAAGAGTTTATCGCAAATGGGGCGCGAGCTGAAATAATGTGT
I-103
GTGCGCAAAGAGTTTATTTCATTTGGTCAATAACCTGTTTATATCGCG
I-104
GTGCGCAAAGAGTTTATTTTAATTGCCCGAAAGACTTCAAAACACTAT
I-114
GTGCGCAAAGAGTTTAATCGGCTGCGAGCATGTAGAAACCTATCATAT
I-115
GTGCGCAAAGAGTTTACCTAATTTACGCTAACGAGCGTCTAATCAATA
I-127
GTGCGCAAAGAGTTTACTAATTTATCTTTCCTTATCATTCATCCTGAA
I-128
GTGCGCAAAGAGTTTATCTTACCAGCCAGTTACAAAATAAATGAAATA
I-130
GTGCGCAAAGAGTTTAATTGAGGGTAAAGGTGAATTATCAATCACCGG
I-131
GTGCGCAAAGAGTTTAAACCAGAGACCCTCAGAACCGCCAGGGGTCAG
I-138
GTGCGCAAAGAGTTTAGGTATTAAGAACAAGAAAAATAATTAAAGCCA
I-141
GTGCGCAAAGAGTTTAGACTTGAGAGACAAAAGGGCGACAAGTTACCA
I-151
GTGCGCAAAGAGTTTATAAGTCCTACCAAGTACCGCACTCTTAGTTGC
I-152
GTGCGCAAAGAGTTTATATTTTGCTCCCAATCCAAATAAGTGAGTTAA
I-162
GTGCGCAAAGAGTTTACAAGCAAGACGCGCCTGTTTATCAAGAATCGC
I-163
GTGCGCAAAGAGTTTATTTTGTTTAAGCCTTAAATCAAGAATCGAGAA
I-172
GTGCGCAAAGAGTTTAAACCTACCGCGAATTATTCATTTCCAGTACAT
I-173
GTGCGCAAAGAGTTTAAAATCAATGGCTTAGGTTGGGTTACTAAATTT
I-175
GTGCGCAAAGAGTTTAAATGCAGACCGTTTTTATTTTCATCTTGCGGG
I-176
GTGCGCAAAGAGTTTAAGGTTTTGAACGTCAAAAATGAAAGCGCTAAT
I-183
GTGCGCAAAGAGTTTAGAGCAAAAACTTCTGAATAATGGAAGAAGGAG
I-186
GTGCGCAAAGAGTTTATCATTACCCGACAATAAACAACATATTTAGGC
I-187
GTGCGCAAAGAGTTTACTTTACAGTTAGCGAACCTCCCGACGTAGGAA

### For Thrombin Loading:

TH-43
AAAAAAAAAAAAAAACAAAAATCATTGCTCCTTTTGATAAGTTTCAT
TH-44
AAAAAAAAAAAAAAAAAAGATTCAGGGGGTAATAGTAAACCATAAAT
TH-57
AAAAAAAAAAAAAAATTTGCCAGATCAGTTGAGATTTAGTGGTTTAA
TH-64
AAAAAAAAAAAAAAAGCAAATATCGCGTCTGGCCTTCCTGGCCTCAG
TH-65
AAAAAAAAAAAAAAATATATTTTAGCTGATAAATTAATGTTGTATAA
TH-78
AAAAAAAAAAAAAAAACCGTTCTAAATGCAATGCCTGAGAGGTGGCA
TH-139
AAAAAAAAAAAAAAAATTATTTAACCCAGCTACAATTTTCAAGAACG
TH-140
AAAAAAAAAAAAAAGAAGGAAAATAAGAGCAAGAAACAACAGCCAT
TH-153
AAAAAAAAAAAAAAAGCCCAATACCGAGGAAACGCAATAGGTTTACC
TH-160
AAAAAAAAAAAAAAATAACCTCCATATGTGAGTGAATAAACAAAATC
TH-161
AAAAAAAAAAAAAAACATATTTAGAAATACCGACCGTGTTACCTTTT
TH-174
AAAAAAAAAAAAAAAAATGGTTTACAACGCCAACATGTAGTTCAGCT

### For Fastening:

F50-48
GGTGGTGGTGGTTGTGGTGGTGGTGGTCTAAAGTTTTGTCGTGAATTGCG
Comp15-48
GTAAAGCTTTTTTTTTTTTACAACCACCACCACC
F50-73
GGTGGTGGTGGTTGTGGTGGTGGTGGTAGAGCTTGACGGGGAAATCAAAA
Comp15-73
TGTAGCATTTTTTTTTTTTACAACCACCACCACC
F50-97
GGTGGTGGTGGTTGTGGTGGTGGTGGCGAGAAAGGAAGGGAACAAACTAT
Comp15-97
TGAGTTTCTTTTTTTTTTTACAACCACCACCACC
F50-120
GGTGGTGGTGGTTGTGGTGGTGGTGGATAGGAACCCATGTACAAACAGTT
Comp15-120
CAAGCCCATTTTTTTTTTTTACAACCACCACCACC
F50-144
GGTGGTGGTGGTTGTGGTGGTGGTGGCACCACCCTCATTTTCCTATTATT
Comp15-144
CCGCCAGCTTTTTTTTTTTACAACCACCACCACC
F50-169
GGTGGTGGTGGTTGTGGTGGTGGTGGCTACATTTTGACGCTCACCTGAAA
Comp15-169
CCCTCAGTTTTTTTTTTTTACAACCACCACCACC

### For Targeting:

T-1
GGTGGTGGTGGTTGTGGTGGTGGTGGTTTTCGATGGCCCACTACGTAAAC
CGTC
T-2
GGTGGTGGTGGTTGTGGTGGTGGTGGTTTTCGGTTTGCGTATTGGGAACG
CGCG
T-11
GGTGGTGGTGGTTGTGGTGGTGGTGGTTTTGACAGCATCGGAACGAACCC
TCAG
T-12
GGTGGTGGTGGTTGTGGTGGTGGTGGATTTTACTTTCAACAGTTTCTGGG
ATTT
T-205
GGTGGTGGTGGTTGTGGTGGTGGTGGTTTTACCAGTAATAAAAGGGATTC
ACCA
T-206
GGTGGTGGTGGTTGTGGTGGTGGTGGTTTTAATCAATATCTGGTCACAAA
TATC
T-215
GGTGGTGGTGGTTGTGGTGGTGGTGGTTTTATAAATCCTCATTAAATGAT
ATTC
T-216
GGTGGTGGTGGTTGTGGTGGTGGTGGTTTTTATAAGTATAGCCCGGCCGT
CGAG

### For Curvature Validation:

44-43
AAAAAAAAAAAAAAAATAGTAAACCATAAATCAAAAATCATTGCTCC
43-42
AAAAAAAAAAAAAAATTTTGATAAGTTTCATTCCATATACATACAGGCAA
GGCAA
56-57
AAAAAAAAAAAAAAAACCCTGACAAAGAAGTTTTGCCAGATCAGTTGAGA
TTTAG
65-64
AAAAAAAAAAAAAAAAATTAATGTTGTATAAGCAAATATCGCGTCTG
64-63
AAAAAAAAAAAAAAAGCCTTCCTGGCCTCAGGAAGATCGGTGCGGGCCTC
TTCGC
77-78
AAAAAAAAAAAAAAATAAACGTTGATATTCAACCGTTCTAAATGCAATGC
CTGAG
140-139
AAAAAAAAAAAAAAAAAGAAACAACAGCCATATTATTTAACCCAGCT
139-138
AAAAAAAAAAAAAAAACAATTTTCAAGAACGGGTATTAAGAACAAGAAAA
ATAAT
152-153
AAAAAAAAAAAAAAACAAATAAGTGAGTTAAGCCCAATACCGAGGAAACG
CAATA
161-160
AAAAAAAAAAAAAAAGACCGTGTTACCTTTTTAACCTCCATATGTGA
160-159
AAAAAAAAAAAAAAAGTGAATAAACAAAATCGCGCAGAGATATCAAAATT
ATTTG
173-174
AAAAAAAAAAAAATTGGGTTACTAAATTTAATGGTTTACAACGCCAACAT
GTA

### REFERENCES FOR SUPPLEMENTAL MATERIAL

1. Castro, C. E., Kilchherr, F., Kim, D N., Shiao, E L., Wauer, T., Wortmann, P., Bathe, M. & Dietz, H. A primer to scaffolded DNA origami. Nat Methods. 8, 221-229 (2011). S39
2. Zhao, Z. et al. Nanocaged enzymes with enhanced catalytic activity and increased stability against protease digestion. Nat Commun. 7, 10619 (2016).
3. Kim, D. N., Kilchherr, F., Dietz, H. & Bathe, M. Quantitative prediction of 3D solution shape and flexibility of nucleic acid nanostructures. Nucleic Acids Res. 40, 2862-2868 (2012).
4. Li, Z., Wang, L., Yan, H. & Liu, Y. Effect of DNA Hairpin Loops on the Twist of Planar DNA Origami Tiles. Langmuir. 28, 1959-1965 (2012).
5. Westendorp, R. G. J., Hottenga, J. J. & Slagboom, P. E. Variation in plasminogen-activator-inhibitor-1 gene and risk of meningococcal septic shock. Lancet. 354, 561-563(1999).
6. Gauberti, M. Martinez de Lizarrondo, S., Orset, C. & Vivien, D. J Thromb Haemost. 12, 409-414(2014).
Swindle, M. M. et al. Swine as models in biomedical research and toxicology testing. Vet. Pathol. 49, 344-356 (2012).
8. Food and Drug Administration, U. S. (2005). Estimating the maximum safe starting dose in initial clinical trials for therapeutics in adult healthy volunteers. Guidance for industry. Rockville, Md. Available at: (http://www.fda.gov/cder/guidance/index.htm).

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A DNA nanostructure nanorobot comprising:
a single stranded DNA scaffold strand of about 5,000 to 10,000 bases in length;
a plurality of staple strands of DNA, wherein each staple strands are about 20 to 40 bases in length, wherein each staple strand has a unique sequence and is hybridized to a specific position on the DNA scaffold strand, wherein the plurality of staple strands hybridized to the DNA scaffold form a sheet having a top surface and a bottom surface; and
one or more fastener strands of DNA, wherein the one or more fastener strands of DNA is capable of fastening the sheet into an origami structure.

2. The DNA nanostructure nanorobot of claim 1, further comprising one or more DNA targeting strands, wherein each targeting strand is operably linked to a targeting moiety.

3. The DNA nanostructure nanorobot of claim 1, wherein the targeting moiety is an aptamer that specifically binds a target molecule.

4. The DNA nanostructure nanorobot of claim 3, wherein the aptamer is specific for nucleolin.

5. The DNA nanostructure nanorobot of any one of claims 1-4, wherein the targeting strand comprises a domain for attaching to the single stranded DNA scaffold strand.

6. The DNA nanostructure nanorobot of any one of claims 1-5, further comprising DNA imaging strands, wherein each imaging strand is operably linked to an imaging agent.

7. The DNA nanostructure nanorobot of claim 6, wherein the imaging agent is a fluorescent dye.

8. The DNA nanostructure nanorobot of any one of claims 1-7, wherein the sheet is a rectangle having four corners and is shaped into a tube-shape.

9. The DNA nanostructure nanorobot of claim 8, wherein the dimension of the rectangular sheet is about 90 nm×about 60 nm×about 2 nm.

10. The DNA nanostructure nanorobot of claim 8 or 9, wherein one or more targeting strands are positioned at one or more corners of the rectangular sheet.

11. The DNA nanostructure nanorobot of any one of claims 8-10, wherein the tube-shaped origami structure has a diameter of about 19 nm.

12. The DNA nanostructure nanorobot of any one of claims 1-11, wherein each of the fastener stands of DNA comprise a first and a second strand of DNA.

13. The DNA nanostructure nanorobot of claim 12, wherein the first and second strand of DNA form a Y-shaped structure.

14. The DNA nanostructure nanorobot of claim 13, wherein the second strand of DNA comprises a domain partially complementary to the first strand.

15. The DNA nanostructure nanorobot of any one of claims 12-14, wherein the first and second strands hybridize to form a 14- to 16-base pair duplex.

16. The DNA nanostructure nanorobot of any one of claims 12-15, wherein the first strand of DNA comprises an aptamer that specifically binds a target molecule and a domain partially complementary to the second strand.

17. The DNA nanostructure nanorobot of claim 16, wherein the aptamer specifically binds nucleolin.

18. The DNA nanostructure nanorobot of claim 17, wherein the aptamer that specifically binds nucleolin is an F50 AS1411 aptamer sequence.

19. The DNA nanostructure nanorobot of claim 18, wherein the oligonucleotide partially complementary to the aptamer comprises a Comp15 DNA sequence.

20. The DNA nanostructure nanorobot of claim 12-19, wherein one of the first or second strand comprises a quencher moiety.

21. The DNA nanostructure nanorobot of claim 20, wherein the other of the first or second strand comprises a fluorophore moiety.

22. The DNA nanostructure nanorobot of claim 21, wherein the Y-shaped structure comprises:
5′-FITC-labeled F50 and 3′-BHQ1-labeled Comp15;
FITC-F50-48 and Comp15-48-Q;
FITC-F50-73 and Comp15-73-Q;
FITC-F50-97 and Comp15-97-Q;
FITC-F50-120 and Comp15-120-Q;
FITC-F50-144 and, Comp15-144-Q; or
FITC-F50-169 and Comp15-169-Q.

23. The DNA nanostructure nanorobot of any one of claims 1-22, wherein the nanorobot further comprises from one to four capture strands.

24. The DNA nanostructure nanorobot of claim 23, wherein the one or more capture strand binds to a poly(A) region in the DNA scaffold strand.

25. The DNA nanostructure nanorobot of any one of claims 1-24, wherein the one or more capture strand is positioned on the top surface of the sheet.

26. The DNA nanostructure nanorobot of any one of claims 1-24, wherein the capture strand is positioned on the bottom surface sheet.

27. The DNA nanostructure nanorobot of any one of claims 23-26, wherein one or more capture strands is operably linked to a therapeutic agent.

28. The DNA nanostructure nanorobot of any one of claims 23-27, wherein the one or more capture strand comprises poly(T).

29. The DNA nanostructure nanorobot of any one of claims 23-28, wherein the one or more capture strand comprises an imaging agent.

30. The DNA nanostructure nanorobot of claim 29, wherein the imaging agent is a fluorescent dye.

31. The DNA nanostructure nanorobot of any one of claims 23-30, wherein the therapeutic agent is a protein.

32. The DNA nanostructure nanorobot of claim 31, wherein the therapeutic agent is thrombin.

33. The DNA nanostructure nanorobot of claim 32, wherein the thrombin is conjugated to the functional strand of DNA by means of a sulfosuccinimidyl-4-(N-maleimidomethyl) cyclohexane-1-carboxylate (sulfo-SMCC) as a bifunctional crosslinker.

34. The DNA nanostructure nanorobot of claim 32 or 33, wherein the nanorobot comprises from one to four thrombin molecules.

35. The DNA nanostructure nanorobot of claim 32 or 33, wherein the nanorobot comprises four thrombin molecules.

36. The DNA nanostructure nanorobot of any one of claims 32-35, wherein the thrombin is operably linked to a fluorescent dye.

37. The DNA nanostructure nanorobot of any one of claims 1-36, wherein each staple strand is about 25 to 35 bases in length.

38. The DNA nanostructure nanorobot of any one of claims 1-36, wherein each staple strand is about 32 bases in length.

39. A DNA nanostructure nanorobot comprising:
a single stranded DNA scaffold strand comprising M13 phage DNA;
a plurality of staple strands 13-204 of DNA, wherein the plurality of staple strands hybridized to the DNA scaffold forms a rectangular sheet having a top surface and a bottom surface, and four corners;
at least six fastener strands of DNA, wherein each fastener strand of DNA is capable of fastening the rectangular sheet into a tube-shaped origami structure;
four DNA capture strands, wherein each capture strand is operably linked to a thrombin; and
at least four targeting strands, wherein each targeting strand is operably linked to an aptamer specific for nucleolin;
a plurality of imaging strands comprising extended ssDNA sequences that hybridized to fluorescent dye-labeled ssDNA.

40. A pharmaceutical composition comprising the DNA nanostructure nanorobot of any one of claims 1-39 and a pharmaceutically acceptable carrier.

41. The pharmaceutical composition of claim 40, further comprising at least one therapeutic agent.

42. The pharmaceutical composition of claim 41, wherein the at least one therapeutic agent is a chemotherapeutic agent.

43. A method of treating a disease or disorder in a subject, comprising administering to the subject a therapeutically effective amount of the DNA nanostructure nanorobot as described in any one of claims 1-39 or a composition as described in any one of claims 40-42.

44. The method of claim 42, wherein the disease or disorder is cancer.

45. The method of claim 43, wherein the cancer is breast cancer, ovarian cancer, melanoma or lung cancer.

46. A method of inhibiting tumor growth in a subject, comprising administering to the subject a therapeutically effective amount of the DNA nanostructure nanorobot as described in any one of claims 1-39 or a composition as described in any one of claims 40-42.

47. The use of the DNA nanostructure nanorobot as described in any one of claims 1-39 or a composition as described in any one of claims 40-42 for the manufacture of a medicament for inducing a tumor necrosis response in a subject.

48. The use of the DNA nanostructure nanorobot as described in any one of claims 1-39 or a composition as described in any one of claims 40-42 for inducing a tumor necrosis response.

49. The use of the DNA nanostructure nanorobot as described in any one of claims 1-39 or a composition as described in any one of claims 40-42 for the manufacture of a medicament for treating a disease or disorder in a subject.

50. The use of the DNA nanostructure nanorobot as described in any one of claims 1-39 or a composition as described in any one of claims 40-42 for the prophylactic or therapeutic treatment a disease or disorder.

51. A kit comprising the DNA nanostructure nanorobot as described in any one of claims 1-39 or a composition as described in any one of claims 40-42 and instructions for administering the DNA nanostructure nanorobot/composition to a subject to induce an immune response or to treat a disease or disorder.

52. The kit of claim 51, further comprising at least one therapeutic agent.
